(19) United States
(12) Patent Application Publication
(10) Pub. No.: US 2017/0101466 A1
(43) Pub. Date: Apr. 13, 2017

(54) ANTI-BLYS ANTIBODIES

(71) Applicants: Masahisa Handa, Berkeley, CA (US); Robert Fick, Chapel Hill, NC (US); Dongxing Zha, Houston, TX (US)

(72) Inventors: Masahisa Handa, Berkeley, CA (US); Robert Fick, Chapel Hill, NC (US); Dongxing Zha, Houston, TX (US)

(21) Appl. No.: 15/315,851

(22) PCT Filed: Jun. 1, 2015
(86) PCT No.: PCT/US15/33454

§ 371 Date: Dec. 2, 2016

Related U.S. Application Data
(60) Provisional application No. 62/007,138, filed on Jun. 3, 2014.

(51) Int. Cl. C07K 16/24; A61K 39/395; C12N 15/81; A61K 45/06

(52) U.S. Cl. CPC C07K 16/241; A61K 45/06; A61K 39/3955; C12N 15/815; C07K 2317/41; A61K 2039/505

(57) ABSTRACT

The present invention provides antibodies and antigen-binding fragments thereof that bind specifically to BlyS, immunoglobulin chains thereof, variants thereof; and method of use thereof, e.g., for the treatment or prevention of inflammatory and/or immune diseases such as systemic lupus erythramatous; as well as polynucleotides encoding the immunoglobulin chains of such antibodies and fragments. Methods for the recombinant expression of immunoglobulin chains are also part of the present invention.

This Application claims the benefit of U.S. Provisional Patent Application No. 62/007,138, filed Jun. 3, 2014; which is herein incorporated by reference in its entirety.

### FIELD OF THE INVENTION

The present invention relates to antibodies and antigen-binding fragments that bind specifically to BLyS and method of use and production thereof.

### BACKGROUND OF THE INVENTION

BLyS, which is also known as B-cell activating factor (BAFF) is a homologous TNF-like cytokine that supports the survival and differentiation of B-cells. BLyS binds to three receptors-B-cell activating factor receptor (BAFF-R), transmembrane activator and calcium-modulator and cyclophilin ligand interactor (TACI), and B-cell maturation antigen (BCMA)—that are expressed on B-cells at different developmental stages. BLyS is expressed as a cell surface protein that is then organized into a trimer, furin cleaved, and released into the circulation. Although standing levels of BLyS are constitutively generated, its expression and secretion can be potentiated by inflammatory cytokines, such as IL-2, TNF-α, and IFN-gamma.

Systemic lupus erythematosus (SLE) is a chronic multisystem autoimmune disease with a broad range of clinical manifestations. SLE can present with many different symptoms, which include photosensitive skin rashes, discoid lesions, arthritis/arthralgia, nephritis, cardiac and pulmonary disease, and CNS disorders. Patients often present with nonspecific symptoms such as fatigue, headache, Raynaud phenomenon, oral ulcers, and mild hair loss, and diagnosis may be delayed. Although SLE is a chronic illness that most often results in debilitated health, it can be life threatening if major organs are affected. It is becoming increasingly clear that accelerated atherosclerosis associated with SLE may contribute to premature mortality.

Numerous factors, both environmental and genetic, contribute to and trigger SLE development and autoantibody production. B cells and BLyS have recently emerged as important factors in the pathology of SLE, regardless of triggering factors. BLyS-BR3 binding and signaling is critical for B cell survival or selection at the TR checkpoint.

Elevated BLyS levels have been implicated in abnormal B cell development, including (but not limited to) autoantibody production, lymphadenopathy development, and lymphomas. By specifically antagonizing the protein, BLyS-targeted agents provide an effective way to control B cell activity and a method to treat SLE.

### SUMMARY OF THE INVENTION

The present invention provides an isolated antibody or antigen-binding fragment thereof that binds specifically to BLyS. Such antibodies and fragments are useful, for example, therapeutically, for treating or preventing SLE. In an embodiment of the invention, the antibodies and fragments of the invention bind specifically to BLyS, e.g., human and/or mouse BLyS, such as SEQ ID NO: 153 and/or 154) (e.g., wherein the isolated antibody or fragment is a monoclonal antibody, a fully human antibody, a mouse antibody, a labeled antibody, a bivalent antibody, a polyclonal antibody, a bispecific antibody, a chimeric antibody, a recombinant antibody, an anti-idiotypic antibody, a humanized antibody or a bispecific antibody, camelized single domain antibody, a diabody, an scfv, an scfv dimer, a dsfv, a (dsfv)₂, a dsFv-dsfv′, a bispecific ds diabody, an Fv, an Fab, an Fab′, an F(ab′)₂, or a domain antibody): (1) comprising a light chain immunoglobulin that comprises: CDR-L1 comprising an amino acid sequence having at least 80% amino acid sequence identity or similarity to an amino acid sequence selected from the group consisting of SEQ ID NOs: 96-114; CDR-L2 comprising an amino acid sequence having at least 80% amino acid sequence identity or similarity to an amino acid sequence selected from the group consisting of SEQ ID NOs: 115-133; and/or CDR-L3 comprising an amino acid sequence having at least 80% amino acid sequence identity or similarity to an amino acid sequence selected from the group consisting of SEQ ID NOs: 134-152; and/or a heavy chain immunoglobulin that comprises: CDR-H1 comprising an amino acid sequence having at least 80% amino acid sequence identity or similarity to an amino acid sequence selected from the group consisting of SEQ ID NOs: 39-57; CDR-H2 comprising an amino acid sequence having at least 80% amino acid sequence identity or similarity to an amino acid sequence selected from the group consisting of SEQ ID NOs: 58-76; and/or CDR-H3 comprising an amino acid sequence having at least 80% amino acid sequence identity or similarity to an amino acid sequence selected from the group consisting of SEQ ID NOs: 77-95; and/or (2) an immunoglobulin light chain that comprises a variable region comprising an amino acid sequence having at least 80% amino acid sequence identity or similarity to the variable region of an immunoglobulin comprising an amino acid sequence selected from the group consisting of SEQ ID NOs: 20-38; and/or an immunoglobulin heavy chain that comprises a variable region comprising an amino acid sequence having at least 80% amino acid sequence identity or similarity to the variable region of an immunoglobulin comprising an amino acid sequence selected from the group consisting of SEQ ID NOs: 1-19; and/or (3) that cross-blocks or is cross-blocked by an antibody or antigen-binding fragment comprising the foregoing. In an embodiment of the invention, the antibody or antigen-binding fragment is linked to a gamma immunoglobulin constant domain (e.g., IgG, IgG1, IgG2, IgG3, IgG4, kappa and/or lambda), e.g., comprising one or more mutations selected from the group consisting of F243X, V264X, S267X and L328X. In an embodiment of the invention, the antibody or antigen-binding fragment is glycosylated, e.g., with an O-linked glycan and/or an N-linked glycan such as SA₍₁₋₄₎Gal₍₁₋₄₎GlcNAc₍₂₋₄₎Man₃GlcNAc₂.

The present invention also provides an injection device (e.g., a syringe comprising a plunger, cylinder and needle) or vessel (e.g., a glass or plastic vial or a chromatography column, e.g., a glass or plastic column) comprising any of the antibodies, fragments, polypeptides and/or polynucleotides discussed herein.

The present invention also provides an isolated host cell (e.g., bacterial cell, mammalian cell, eukaryotic cell, lower eukaryotic cell, yeast cell or fungal cell) comprising any of the antibodies, fragments, polypeptides and/or polynucleotides discussed herein. In an embodiment of the invention, the host cell is Pichia, such as Pichia pastoris. For example, in an embodiment of the invention, the host cell is a Pichia pastoris host cell comprising the genotype:
ura5Δ::ScSUC2 och1Δ::lacZ bmt2Δ::lacZ/KIMNN2-2
mnn4L1Δ::lacZ/MmSCL35A3 pno1Δ mnn4Δ::lacZ
ADE1::lacZ/NA10/MmSLC35A3/FB8
his1Δ::lacZ/ScGAL10/XB33/DmUGT
arg1Δ::HIS1/KD53/TC54
bmt4Δ::lacZ bmt1Δ::lacZ bmt3Δ::lacZ
TRP2::ARG1/MmCST/HsGNE/HsCSS/HsSPS/MmST6-33
ste13Δ::lacZ-URA5-lacZ/TrMDS1 dap2Δ::NatR
TRP5::HygRMmCST/HsGNE/HsCSS/HsSPS/MmST6-33
att1Δ::ScARR3/LmSTT3D.

The present invention also provides an isolated polypeptide that comprises an amino acid sequence having at least 80% sequence similarity or identity to an amino acid sequence selected from the group consisting of SEQ ID NOs: 1-152; or an isolated polynucleotide (e.g., in a vector such as a viral vector or plasmid) that encodes such a polypeptide.

The present invention also provides a pharmaceutical composition comprising any antibody, antigen-binding fragment thereof (e.g., any of 6C10 or 10C11 or 6A11 or 5B7 or 1B7 or 5E12 or 5G10 or 8D12 or 2C5 or 6F5 or 8A12 or 1B5 or 10B1 or 4A2 or 14F11 or 9F8 or 5D10 or 1D6 or 1F4), polypeptide or polynucleotide discussed herein along with a pharmaceutically acceptable carrier (e.g., comprising polysorbate-80). The present invention also provides a composition comprising any antibody, antigen-binding fragment thereof (e.g., any of 6C10 or 10C11 or 6A11 or 5B7 or 1B7 or 5E12 or 5G10 or 8D12 or 2C5 or 6F5 or 8A12 or 1B5 or 10B1 or 4A2 or 14F11 or 9F8 or 5D10 or 1D6 or 1F4), polypeptide or polynucleotide and a further chemotherapeutic agent such as, for example, a member selected from the group consisting of: an anti-inflammatory drug (e.g., a steroid or a non-steroidal anti-inflammatory drug (NSAID)), belimumab, tadalumab, denosumab, aspirin, diclofenac, diflunisal, etodolac, fenoprofen, floctafenine, flurbiprofen, ibuprofen, indomethacin, ketoprofen, ketorolac, meclofenamate, mefenamic acid, meloxicam, nabumetone, naproxen, oxaprozin, phenylbutazone, piroxicam, salsalate, sulindac, tenoxicam, tiaprofenic acid, tolmetin, betamethasone benzoate, betamethasone valerate, clobetasol propionate, desoximetasone, fluocinolone acetonide, flurandrenolide, a topical steroid, alclometasone dipropionate, aloe vera, amcinonide, amcinonide, anthralin, betamethasone dipropionate, betamethasone valerate, calcipotriene, clobetasol propionate, coal tar, Dead Sea salts, desonide, desonide; betamethasone valerate, desoximetasone, diflorasone diacetate, epsom salts, fluocinolone acetonide, fluocinonide, flurandrenolide, fluticasone propionate, halcinonide, halobetasol propionate, hydrocortisone valerate, hydrocortisone, mometasone furoate, oilated oatmeal, petroleum jelly, prednicarbate, salicylic acid, tazarotene, triamcinolone acetonide, a mixture of hydrocortisone, dexamethasone, methylprednisolone and prednisolone, alefacept, etanercept, cyclosporine, methotrexate, acitretin, isotretinoin, hydroxyurea, mycophenolate mofetil, sulfasalazine, 6-Thioguanine, anakinra, injectable gold, penicillamine, azathioprine, chloroquine, hydroxychloroquine, sulfasalazine, oral gold, auranofin, gold sodium thiomalate, aurothioglucose, mesalamine, sulfasalazine, budesonide, metronidazole, ciprofloxacin, azathioprine, 6-mercaptopurine or dietary supplementation of calcium, folate, vitamin B12, celecoxib, rofecoxib, valdecoxib, lumiracoxib, etoricoxib, efalizumab, adalimumab, infliximab, rituximab, tocilizumab, and ABX-IL8.

The present invention also provides a method for producing any polypeptide, antibody or antigen-binding fragment thereof discussed herein comprising introducing a polynucleotide encoding a polypeptide that comprises an amino acid sequence having at least 80% sequence similarity or identity to an amino acid sequence selected from the group consisting of SEQ ID NOs: 1-152 into a host cell; culturing the host cell under conditions favorable to expression of the chains; and, optionally, isolating the chains. Such a host cell, comprising the polypeptide, is part of the present invention.

The present further provides a method for treating or preventing an autoimmune disorder or an inflammatory disorder (e.g., appendicitis, peptic ulcer, gastric ulcer and duodenal ulcer, peritonitis, liver steatosis, pancreatitis, inflammatory bowel disease, colitis, ulcerative colitis, pseudomembranous colitis, acute colitis, ischemic colitis, diverticulitis, epiglottitis, achalasia, cholangitis, cholecystitis, coeliac disease, hepatitis, Crohn's disease, enteritis, Whipple's disease, asthma, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, and urethritis, bronchitis, emphysema, rhinitis, fibrosis, cystic fibrosis, pneumonitis, adult respiratory distress syndrome, pneumoultramicroscopicsilicovolcanoconiosis, alvealitis, bronchiolitis, pharyngitis, pleurisy, sinusitis, dermatitis, atopic dermatitis, dermatomyositis, sunburn, urticaria warts, wheals, stenosis, restenosis, vasulitis, angiitis, endocarditis, arteritis, atherosclerosis, thrombophlebitis, pericarditis, myocarditis, myocardial ischemia, periarteritis nodosa, rheumatic fever, meningitis, encephalitis, multiple sclerosis, neuritis, neuralgia, uveitis, arthritides and arthralgias, osteomyelitis, fasciitis, Paget's disease, gout, periodontal disease, rheumatoid arthritis (RA), synovitis, myasthenia gravis, thryoiditis, systemic lupus erythematosus (SLE), goodpasture's syndrome, behcets's syndrome, allograft rejection, graft-versus-host disease, B-cell lymphoma, non-hodgkins lymphoma, leukemia, chronic lymphocytic leukemia, granulomatosis with polyangiitis (GPA; Wegener's granulomatosis), Churg-Strauss syndrome, an ANCA-associated vasculitide and/or microscopic polyangiitis), in a subject, comprising administering a therapeutically effective amount of any antibody or antigen-binding fragment thereof discussed herein (e.g., any of 6C10 or 10C11 or 6A11 or 5B7 or 1B7 or 5E12 or 5G10 or 8D12 or 2C5 or 6F5 or 8A12 or 1B5 or 10B1 or 4A2 or 14F11 or 9F8 or 5D10 or 1D6 or 1F4) to the subject.

### BRIEF DESCRIPTION OF THE FIGURES

FIG. 1. Representative anti-BLyS mAb expression plasmid map pGLY13667.

FIG. 2. Characterization of sialylated and desialylated (desialylated by neuraminidase treatment) anti-BLyS mAb 5B7.

FIG. 3. SDS-PAGE analysis of anti-BLyS mAbs under non-reducing conditions (lanes to the left) or reducing conditions (lanes to the right).

FIG. 4 (a)-(c). Comparison of Pichia produced alpha 2,6 sialylated 5B7 with HEK293 produced 5B7 in mouse splenic B cell proliferation assay. A first (a) and second trial (b) of the assays were performed and a comparison of the trial results was performed (c).

### DETAILED DESCRIPTION OF THE INVENTION

The present invention provides, inter alia, isolated antibodies and antigen-binding fragments thereof which were isolated from a fully human scFv phage library. Antibodies of the present invention exhibited various characteristics including binding with high affinity to BLyS from human, mouse and cynomolgous monkey, inhibition of binding of BLyS to the BLyS receptor BR3, as well as therapeutic activity for the treatment of diseases such as systemic lupus erythematosus (SLE). An advantageous property of the anti-BLyS antibodies and antigen-binding fragments thereof of the present invention is the ability to inhibit mouse BLyS-induced B-cell proliferation. As shown in Table 6, benlysta and tabalumab exhibited no detectable activity in the tests conducted herein whereas the instant antibodies exhibited potent inhibition activity. See also Baker et al., Arthritis & Rheumatism. 48(11): 3253-3265 (2003). This activity is helpful in the study and characterization of BLyS and of the instant antibodies and antigen-binding fragments and their roles in B-cell proliferation and, for example, SLE. Furthermore, antibodies and antigen-binding fragments thereof of the present invention exhibit an ability to bind to membrane-bound BLyS whereas, for example, belimumab does not bind to membrane-bound BLyS (BENLYSTA Product Monograph).

### Molecular Biology

In accordance with the present invention there may be employed conventional molecular biology, microbiology, and recombinant DNA techniques within the skill of the art. Unless otherwise defined herein, scientific and technical terms used in connection with the present invention shall have the meanings that are commonly understood by those of ordinary skill in the art. Further, unless otherwise required by context, singular terms shall include the plural and plural terms shall include the singular. Generally, nomenclatures used in connection with, and techniques of biochemistry, enzymology, molecular and cellular biology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. See, e.g., James M. Cregg (Editor), Pichia Protocols (Methods in Molecular Biology), Humana Press (2010), Sambrook et al. Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lane, Antibodies: A Laboratory Manual, Cold Spring Harbor Laboratory Press, Cold Spring Harbor, N.Y. (1990); Taylor and Drickamer, Introduction to Glycobiology, Oxford Univ. Press (2003); Worthington Enzyme Manual, Worthington Biochemical Corp., Freehold, N.J.; Handbook of Biochemistry: Section A Proteins, Vol I, CRC Press (1976); Handbook of Biochemistry: Section A Proteins, Vol II, CRC Press (1976); Essentials of Glycobiology, Cold Spring Harbor Laboratory Press (1999), Animal Cell Culture (R. I. Freshney, ed. (1986)); Immobilized Cells And Enzymes (IRL Press, (1986)); B. Perbal, A Practical Guide To Molecular Cloning (1984).

A “polynucleotide” or “nucleic acid” includes DNA and RNA in single stranded form, double-stranded form or otherwise. A “polynucleotide sequence” or “nucleotide sequence” is a series of nucleotide bases (also called “nucleotides”) in a nucleic acid, such as DNA or RNA, and means a series of two or more nucleotides. Any polynucleotide comprising a nucleotide sequence set forth herein forms part of the present invention.

A “protein”, “peptide” or “polypeptide” (e.g., a heterologous polypeptide such as an immunoglobulin heavy chain and/or light chain) includes a contiguous string of two or more amino acids. Any polypeptide comprising an amino acid sequence set forth herein forms part of the present invention.

A “protein sequence”, “peptide sequence” or “polypeptide sequence” or “amino acid sequence” refers to a series of two or more amino acids in a protein, peptide or polypeptide.

The term “isolated” polynucleotide, polypeptide, antibody or antigen-binding fragment includes such a polynucleotide, polypeptide, antibody or antigen-binding fragment, respectively, which is partially or fully separated from other components that are normally found in cells or in recombinant DNA expression systems or any other contaminant. These components include, but are not limited to, cell membranes, cell walls, ribosomes, polymerases, serum components and extraneous genomic sequences. The scope of the present invention includes the isolated polynucleotides, polypeptides, antibodies AND antigen-binding fragments set forth herein.

An isolated polynucleotide, polypeptide, antibody or antigen-binding fragment will, preferably, be an essentially homogeneous composition of molecules but may contain some heterogeneity.

In general, a “promoter” or “promoter sequence” is a DNA regulatory region capable of binding an RNA polymerase in a cell (e.g., directly or through other promoter-bound proteins or substances) and initiating transcription of a coding sequence to which it operably links. Polynucleotides encoding an immunoglobulin of the present invention, operably linked to a promoter, form part of the present invention. Also, an isolated host cell comprising a heterologous polynucleotide (e.g., encoding an immunoglobulin polypeptide) operably linked to a promoter also forms part of the present invention.

A coding sequence (e.g., of a heterologous polynucleotide, e.g., reporter gene or immunoglobulin heavy and/or light chain) is “operably linked to”, “under the control of”, “functionally associated with” or “operably associated with” a transcriptional and translational control sequence (e.g., a promoter of the present invention) when the sequence directs RNA polymerase mediated transcription of the coding sequence into RNA, preferably mRNA, which then may be RNA spliced (if it contains introns) and, optionally, translated into a protein encoded by the coding sequence.

The present invention includes vectors or cassettes which comprise polynucleotides encoding an immunoglobulin polypeptide of the present invention. Vectors containing a heterologous polynucleotide encoding a heterologous polypeptide can also be used in host cells for production of an immunogloubulin polypeptide. The term “vector” includes a vehicle (e.g., a plasmid) by which a DNA or RNA sequence can be introduced into a host cell, so as to transform the host and, optionally, promote expression and/or replication of the introduced sequence. Suitable vectors for use herein include plasmids, integratable DNA fragments, and other vehicles that may facilitate introduction of the nucleic acids into the genome of a host cell (e.g., Pichia pastoris). Plasmids are the most commonly used form of vector but all other forms of vectors which serve a similar function and which are, or become, known in the art are suitable for use herein. See, e.g., Pouwels, et al., Cloning Vectors: A Laboratory Manual, 1985 and Supplements, Elsevier, N.Y., and Rodriguez et al. (eds.), Vectors: A Survey of Molecular Cloning Vectors and Their Uses, 1988, Buttersworth, Boston, Mass. Such vectors optionally include a secretion signal (e.g., alpha-mating factor pre-pro leader sequence (e.g., alpha-mating factor pre-pro leader sequence (α-MF)) operably linked to the immunoglobulin polynucleotide. Also, an isolated host cell comprising a vector that includes an immunoglobulin polynucleotide, e.g., operably linked to a promoter, also forms part of the present invention.

A polynucleotide (e.g., a heterologous polynucleotide, e.g., encoding an immunoglobulin heavy chain and/or light chain), operably linked to a promoter, may be expressed in an expression system. The term “expression system” means a host cell and compatible vector which, under suitable conditions, can express a protein or nucleic acid which is carried by the vector and introduced to the host cell. Common expression systems include fungal host cells (e.g., Pichia pastoris) and plasmid vectors, insect host cells and Baculovirus vectors, and mammalian host cells and vectors.

The term methanol-induction refers to increasing expression of a polynucleotide (e.g., a heterologous polynucleotide) operably linked to a methanol-inducible promoter in a host cell of the present invention by exposing the host cells to methanol. A host cell containing a polynucleotide, e.g., encoding an immunoglobulin, operably linked to a methanol-inducible promoter forms part of the present invention. Methods for inducing expression of a polynucleotide encoding a polypeptide of the present invention operably linked to a methanol-inducible promoter by exposing a host cell comprising the promoter construct to methanol, and culturing the cell under conditions favorable to expression of the encoded heterologous polypeptide form part of the present invention. Such polynucleotides, themselves, and host cells comprising such polynucleotides form part of the present invention as well.

The following references regarding the BLAST algorithm are herein incorporated by reference: BLAST ALGORITHMS: Altschul, S. F., et al., J. Mol. Biol. (1990) 215:403-410; Gish, W., et al., Nature Genet. (1993) 3:266-272; Madden, T. L., et al., Meth. Enzymol. (1996) 266:131-141; Altschul, S. F., et al., Nucleic Acids Res. (1997) 25:3389-3402; Zhang, J., et al., Genome Res. (1997) 7:649-656; Wootton, J. C., et al., Comput. Chem. (1993) 17:149-163; Hancock, J. M., et al., Comput. Appl. Biosci. (1994) 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., J. Mol. Biol. (1991) 219:555-565; States, D. J., et al., Methods (1991) 3:66-70; Henikoff, S., et al., Proc. Natl. Acad. Sci. USA (1992)89:10915-10919; Altschul, S. F., et al., J. Mol. Evol. (1993) 36:290-300; ALIGNMENT STATISTICS: Karlin, S., et al., Proc. Natl. Acad. Sci. USA (1990) 87:2264-2268; Karlin, S., et al., Proc. Natl. Acad. Sci. USA (1993) 90:5873-5877; Dembo, A., et al., Ann. Prob. (1994) 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

Sequence identity refers to exact matches between amino acid and nucleotide sequences that are compared. Sequence similarity refers to matches between amino acid sequences wherein the matched amino acid residues are within the same amino acid family (e.g., basic, non-polar or aromatic).

The present invention includes any host cell that comprises a polypeptide of the present invention (SEQ ID NOs: 1-152) or any anti-BLyS antibody immunoglobulin heavy chain and/or light chain of the present invention or any polypeptide comprising one or more CDRs thereof (e.g., 6C10, 10C11, 6A11, 5B7, 1B7, 5E12, 5G10, 8D12, 2C5, 6F5, 8A12, 1B5, 10B1, 4A2, 14F11, 9F8, 5D10, 1D6 or 1F4), for example, V_{H} and/or V_{L} thereof. The present invention also includes any lysate or fraction of such a host cell that includes such antibody, fragment, polypeptide and/or polynucleotide.

In an embodiment of the invention, a host cell is eukaryotic or prokaryotic (e.g., bacteria such as E. coli). In an embodiment of the invention, a eukaryotic cell is a mammalian cell or a lower eukaryotic. In an embodiment of the invention, an isolated host cell of the present invention belongs to the Fungi kingdom, for example, in an embodiment of the invention, the fungal host cell is any yeast such as a budding yeast and/or a fission yeast. In an embodiment of the invention, a host cell is selected from the group consisting of any Pichia cell, such as Pichia pastoris, Pichia angusta (Hansenula polymorpha), Pichia flnlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis or Pichia methanolica; Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum and Neurospora crassa.

### BLyS

The present invention comprises antibodies and antigen-binding fragments thereof that bind specifically to BLyS as well as complexes between BLyS and the antibodies or fragments.

In an embodiment of the invention, human BLyS comprises the amino acid sequence:

(SEQ ID NO: 153)

MDDSTEREQS RLTSCLKKRE EMKLKECVSI LPRKESPSVR
SSKDGKLLAA TLLLALLSCCLTVVSFYQVA ALQGDLASLR
AELQGHHAEK LPAGAGAPKA GLEEAPAVTA
GLKIFEPPAPGEGNSSQNSR NKRAVQGPEE TVTQDCLQLI
ADSETPTIQK GSYTFVPWLL SFKRGSALEEKENKILVKET
GYFFIYGQVL YTDKTYAMGH LIQRKKVHVF GDELSLVTLF
RCIQNMPETLPNNSCYSAGI AKLEEGDELQ LAIPRENAQI
SLDGDVTFFG ALKLL

In an embodiment of the invention, mouse BLyS comprises the amino acid sequence:

(SEQ ID NO: 154)

MDESAKTLPPPCLCFCSEKGEDMKVGYDPITPQKEEGAWFGICRDGRLLA
ATLLLALLSSSFTAMSLYQLAALQADLMNLRMELQSYRGSATPAAAGAPE
LTAGVKLLTPAAPRPHNSSRGHRNRRAFQGPEETEQDVDLSAPPAPCLPG
CRHSQHDDNGMNLRNIIQDCLQLIADSDTPTIRKGTYTFVPWLLSFKRGN
ALEEKENKIVVRQTGYFFIYSQVLYTDPIFAMGHVIQRKKVHVFGDELSL
VTLFRCIQNMPKTLPNNSCYSAGIARLEEGDEIQLAIPRENAQISRNGDD
TFFGALKLL

### Antibodies

The present invention provides antibodies and antigen-binding fragments thereof that bind specifically (e.g., with a K_{D} of about 10⁻⁷M or a lower number, e.g., 10⁻⁸M, 10⁻⁹M, 10⁻¹⁰M, 10⁻¹¹M, 10⁻¹²M) to BLyS (e.g., soluble or membrane-bound). In an embodiment of the invention, the antibody or antigen-binding fragment thereof comprises an immunoglobulin polypeptide of 6C10, 10C11, 6A11, 5B7, 1B7, 5E12, 5G10, 8D12, 2C5, 6F5, 8A12, 1B5, 10B1, 4A2, 14F11, 9F8, 5D10, 1D6 or 1F4 or a “sequence variant” thereof.

“Sequence variant” immunoglobulins of the present invention comprise amino acid sequences which are at least about 70% identical or similar, preferably at least about 80% identical or similar, more preferably at least about 90% identical or similar and most preferably at least about 95% identical or similar (e.g., 95%, 96%, 97%, 98%, 99%, 100%) to a 6C10, 10C11, 6A11, 5B7, 1B7, 5E12, 5G10, 8D12, 2C5, 6F5, 8A12, 1B5, 10B1, 4A2, 14F11, 9F8, 5D10, 1D6 or 1F4 immunoglobulin heavy or light chain or variable region thereof or CDR thereof (e.g., SEQ ID NOs: 1-152) when the comparison is performed by a BLAST algorithm wherein the parameters of the algorithm are selected to give the largest match between the respective sequences over the entire length of the respective reference sequences.

Isolated heavy chain or light chain immunoglobulin polypeptides from the 6C10, 10C11, 6A11, 5B7, 1B7, 5E12, 5G10, 8D12, 2C5, 6F5, 8A12, 1B5, 10B1, 4A2, 14F11, 9F8, 5D10, 1 D6 or 1F4 antibodies set forth herein or a variable region or CDR thereof; and sequence variants thereof are also part of the present invention.

Any isolated polypeptide comprising one or more heavy chain CDRs (e.g., 3) and/or light chain CDRs (e.g., 3) of a 6C10, 10C11, 6A11, 5B7, 1B7, 5E12, 5G10, 8D12, 2C5, 6F5, 8A12, 1B5, 10B1, 4A2, 14F11, 9F8, 5D10, 1D6 or 1F4 immunoglobulin heavy or light chain; or a sequence variant thereof is part of the present invention.

An isolated antibody or antigen-binding fragment thereof comprising one or more (e.g., 3) heavy chain CDRs and/or one or more (e.g., 3) light chain CDRs of a 6C10, 10C11, 6A11, 5B7, 1B7, 5E12, 5G10, 8D12, 2C5, 6F5, 8A12, 1B5, 10B1, 4A2, 14F11, 9F8, 5D10, 1 D6 or 1F4 immunoglobulin heavy or light chain; or a sequence variant thereof is part of the present invention.

The present invention includes polynucleotides encoding any polypeptide of the present invention, e.g., any of SEQ ID NOs: 1-152, e.g., a 6C10, 10C11, 6A11, 5B7, 1B7, 5E12, 5G10, 8D12, 2C5, 6F5, 8A12, 1B5, 10B1, 4A2, 14F11, 9F8, 5D10, 1D6 or 1F4 immunoglobulin heavy or light chain as well as sequence variant polynucleotides which hybridize thereto as well as polypeptides encoded thereby. Preferably, the polynucleotides hybridize under low stringency conditions, more preferably under moderate stringency conditions and most preferably under high stringency conditions. A polynucleotides is “hybridizable” to another polynucleotides, such as a cDNA, genomic DNA, or RNA, when a single stranded form of the polynucleotides can anneal to the other polynucleotides under the appropriate conditions of temperature and solution ionic strength (see Sambrook et al). The conditions of temperature and ionic strength determine the “stringency” of the hybridization. In an embodiment of the invention, low stringency hybridization conditions are 55° C., 5×SSC, 0.1% SDS, 0.25% milk, and no formamide at 42° C.; or 30% formamide, 5×SSC, 0.5% SDS at 42° C. In an embodiment of the invention, moderate stringency hybridization conditions are similar to the low stringency conditions except the hybridization is carried out in 40% formamide, with 5× or 6×SSC at 42° C. In an embodiment of the invention, high stringency hybridization conditions are similar to low stringency conditions except the hybridization conditions are carried out in 50% formamide, 5× or 6×SSC and, optionally, at a higher temperature (e.g., higher than 42° C.: 57° C., 59° C., 60° C., 62° C., 63° C., 65° C. or 68° C.). In general, SSC is 0.15M NaCl and 0.015M Na-citrate.

Sequence variants of antibodies and antigen-binding fragments and polypeptides of the present invention include those where an asparagine deamination motif has been mutated. The deamidation of asparagine may occur at N-G motif and result in the creation of an aspartic acid or isoaspartic acid residue that negatively affects antigen-binding. Thus, where an N-G appears in an antibody or fragment or polypeptide set forth herein, the invention includes the corresponding sequence variant wherein N-G is mutated to N-X; wherein X is any amino acid other than glycine.

Sequence variants of antibodies and antigen-binding fragments and polypeptides of the present invention include those where an aspartate isomerization motif has been mutated. The isomerization of aspartate may occur on D-G sequences and result in the creation of an isoAsp-Gly residue or a cyclic imide intermediate that negatively affects antigen-binding. Cacia et al., Biochemistry 35: 1897-1903 (1996). Thus, where a D-G appears in an antibody or fragment or polypeptide set forth herein, the invention includes the corresponding sequence variant wherein D-G is mutated to D-X; wherein X is any amino acid other that glycine.

Sequence variants of antibodies and antigen-binding fragments and polypeptides of the present invention include those where a methionine or tryptophan has been mutated. The oxidation of methionine or tryptophan may occur and negatively affects antigen-binding. Thus, where n methionine or tryptophan appears in an antibody or fragment or polypeptide set forth herein, the invention includes the corresponding sequence variant wherein the methionine or tryptophan is mutated to any other amino acid. See e.g., Wei et al. Anal Chem. 79(7):2797-2805 (2007).

Sequence variants of antibodies and antigen-binding fragments and polypeptides of the present invention include those where a cysteine has been mutated. The cysteinylation of a free cysteine may occur and negatively affects antigen-binding. Thus, where a cysteine appears in an antibody or fragment or polypeptide set forth herein, the invention includes the corresponding sequence variant wherein the cysteine is mutated to any other amino acid. See e.g., Banks et al. J Pharm Sci. 97(2):775-790 (2008).

The present invention includes anti-BLyS antibodies and antigen-binding fragments thereof that cross-block the binding of one of the antibodies or fragments described herein (e.g., 6C10, 10C11, 6A11, 5B7, 1B7, 5E12, 5G10, 8D12, 2C5, 6F5, 8A12, 1B5, 10B1, 4A2, 14F11, 9F8, 5D10, 1D6, 1F4 and sequence variants thereof) and/or are cross-blocked from binding BLyS by one of the antibodies or fragments described herein (e.g., 6C10, 10C11, 6A11, 5B7, 1B7, 5E12, 5G10, 8D12, 2C5, 6F5, 8A12, 1B5, 10B, 4A2, 14F11, 9F8, 5D10, 1D6, 1 F4 and sequence variants thereof). Such cross-blocking antibodies and antigen-binding fragments thereof can be identified based on their ability to cross-compete with 6C10, 10C11, 6A11, 5B7, 1B7, 5E12, 5G10, 8D12, 2C5, 6F5, 8A12, 1B5, 10B, 4A2, 14F11, 9F8, 5D10, 1D6, 1F4 in standard binding assays (e.g., BIACore, ELISA, flow cytometry). For example, standard ELISA assays can be used in which a recombinant BLyS (e.g., human BLyS) protein is immobilized on the plate, one of the antibodies is fluorescently labeled and the ability of non-labeled antibodies to compete off the binding of the labeled antibody is evaluated. Additionally or alternatively, BIAcore analysis can be used to assess the ability of the antibodies to cross-compete. The ability of a test antibody to inhibit the binding of, for example, 6C10, 10C11, 6A11, 5B7, 1B7, 5E12, 5G10, 8D12, 2C5, 6F5, 8A12, 1B5, 101, 4A2, 14F11, 9F8, 5D10, 1D6 and/or 1F4 to BLyS (e.g., human BLyS) demonstrates that the test antibody can compete with 6C10, 10C11, 6A11, 5B7, 1B7, 5E12, 5G10, 8D12, 2C5, 6F5, 8A12, 1B5, 10B1, 4A2, 14F11, 9F8, 5D10, 1D6 and/or 1F4 for binding to BLyS (e.g., human BLyS) and thus, may, in some cases, bind to the same epitope on BLyS (e.g., human BLyS) as 6C10, 10C11, 6A11, 5B7, 1B7, 5E12, 5G10, 8D12, 2C5, 6F5, 8A12, 1B5, 10B1, 4A2, 14F11, 9F8, 5D10, 1D6 and/or 1F4. Antibodies and fragments that bind to the same epitope as any of the anti-BLyS antibodies or fragments of the present invention also form part of the present invention.

Amino acid sequences of 19 anti-BLyS mAbs, 6C10, 10C11, 6A11, 5B7, 1B7, 5E12, 5G10, 8D12, 2C5, 6F5, 8A12, 1B5, 10B1, 4A2, 14F11, 9F8, 5D10, 1D6 or 1F4, are set forth below.

The present invention also includes antibodies and antigen-binding fragments thereof comprising any combination of immunoglobulin light and heavy chains or variable regions thereof, each selected independently from 6C10, 10C11, 6A11, 5B7, 1B7, 5E12, 5G10, 8D12, 2C5, 6F5, 8A12, 1B5, 10B1, 4A2, 14F11, 9F8, 5D10, 1D6, 1F4 and sequence variants thereof, as discussed herein.

The present invention also includes antibodies and antigen-binding fragments and polypeptides thereof comprising 3 light chain CDRs and/or 3 heavy chain CDRs, each independently selected from 6C10, 10C11, 6A11, 5B7, 1B7, 5E12, 5G10, 8D12, 2C5, 6F5, 8A12, 1B5, 10B1, 4A2, 14F11, 9F8, 5D10, 1D6, 1F4 and sequence variants thereof as discussed herein.

The light chain and heavy chain sequences of antibodies 6C10, 10C11, 6A11, 5B7, 1B7, 5E12, 5G10, 8D12, 2C5, 6F5, 8A12, 1B5, 10B1, 4A2, 14F11, 9F8, 5D10, 1D6 and 1F4 are set forth below wherein the CDRs are underscored with a solid line and wherein the immunoglobulin constant region is underscored with a broken line. The sequences lacking a broken underscore line are the variable regions of the antibody chains.

In an embodiment of the invention, a sequence variant of an antibody or antigen-binding fragment thereof that binds specifically to BLyS is characterized by any one or more of several functional characteristics including, but not limited to:
Lack of detectable binding to APRIL;
Inhibits binding of BLys to BR3, BCMA, and/or TACI;
K_{D} for binding to human BLyS of about 1040 pM or a lower number;
K_{D} for binding to mouse BLyS of about 732 pM or a lower number;
Inhibits proliferation of B-cells (e.g., splenic B-cells, e.g., from mouse or human).

The 6C10 heavy chain immunoglobulin comprises the amino acid sequence:

(SEQ ID NO: 1)
E V Q L V Q S G A E V K K P G A S V K V S C K A S G
Y T F T S Y G I S W V R Q A P G Q G L E W M G W I S
A Y N G N T N Y A Q K L Q G R V T M T T D T S T S T
A Y M E L R S L R S D D T A V Y Y A R T Y Y Y D I L

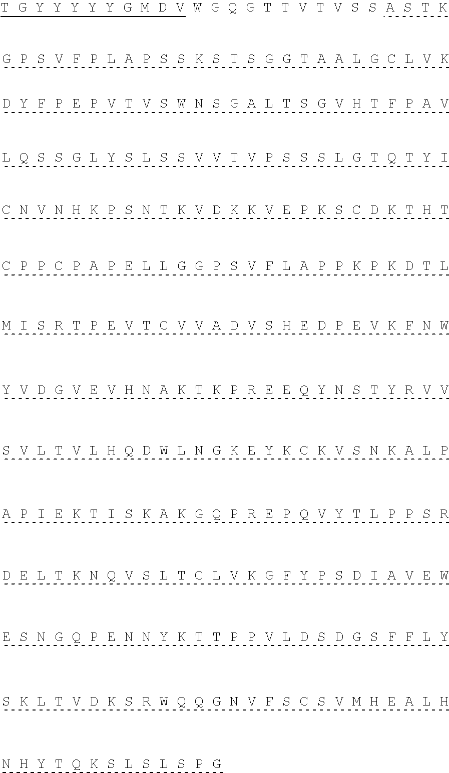

(SEQ ID NO: 39)
CDR-H1: G Y T F T S Y G I S_
(SEQ ID NO: 58)
CDR-H2: I S A Y N G N T N Y A Q K L Q G
(SEQ ID NO: 77)
CDR-H3: T Y Y D I L T G Y Y Y Y Y G M D V

The 6C10 light chain immunoglobulin comprises the amino acid sequence:

(SEQ ID NO: 20)
E I V L T Q S P G T L S L S P G E R A M L S C R A S
Q S V G N Y L A W Y Q Q K P G Q A P R L L I Y D A S
N R A T G I P A R F S G S G S G T D F T L T I N S L
E P E D F A V Y Y C Q Q R S N W P L T F G G G T K V

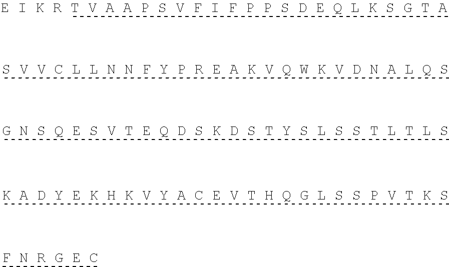

(SEQ ID NO: 96)
CDR-L1: R A S Q S V G N Y L A
(SEQ ID NO: 115)
CDR-L2: D A S N R A T
(SEQ ID NO: 134)
CDR-L3: Q Q R S N W P L T

The 10C11 heavy chain immunoglobulin comprises the amino acid sequence:

(SEQ ID NO: 2)
E V Q L V Q S G A E V K K P G S S V K V S C K A S G
G T F S S Y A I S W V R Q A P G Q G L E W M G R I I
P I L G I A N Y A Q K F Q G R V T I T A D K S T S T
A Y M E L S R L R S D D T A V Y Y C A R D R F Y D I

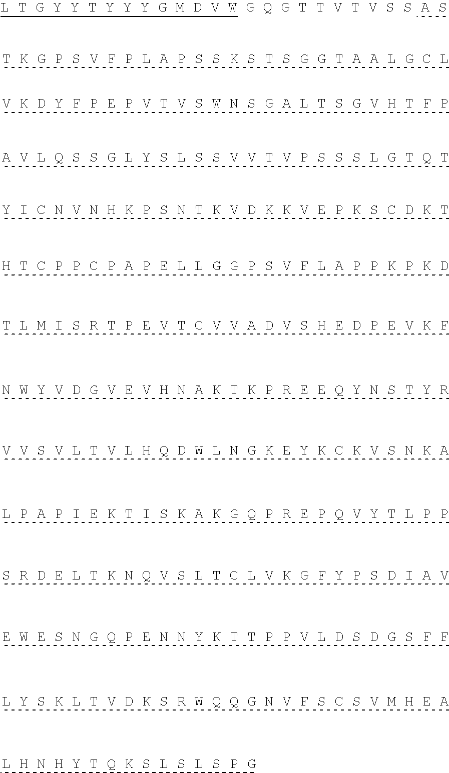

(SEQ ID NO: 40)
CDR-H1: G G T F S S Y A I S
(SEQ ID NO: 59)
CDR-H2: R I I P I L G I A N Y A Q K F Q G R
(SEQ ID NO: 78)
CDR-H3: D R F Y D I L T G Y Y T Y Y Y G M D V W

The 10C11 light chain immunoglobulin comprises the amino acid sequence:

(SEQ ID NO: 21)
D I V L T Q S P G T L S F S P G E R A T L S C R A S
Q N V A N N F L A W F Q H S P G Q A P R L L I Y D A
S N R A T G I P A R F S G S G S G T D F T L T I S S
L Q T E D V A V Y Y C Q Q Y Y N L P Y T F G Q G T K

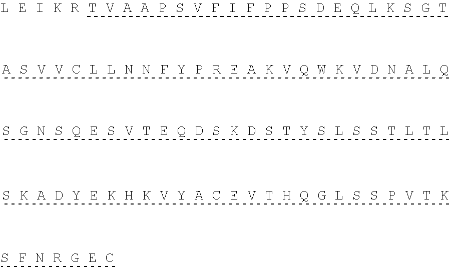

(SEQ ID NO: 97)
CDR-L1: R A S Q N V A N N F L A
(SEQ ID NO: 116)
CDR-L2: D A S N R A T
(SEQ ID NO: 135)
CDR-L3: Q Q Y Y N L P Y T

The 6A11 heavy chain immunoglobulin comprises the amino acid sequence:

(SEQ ID NO: 3)
Q I Q L V Q S G T E V K K P G A S V K V S C K A S G
Y T F T D Y Y M H W V R Q A P G Q G L E W M G R I N
P N S G G T N Y A Q K F Q G R V T M T R D T S I S T
A Y M E L S R L R S D D T F V Y Y C A R E G Y D F L

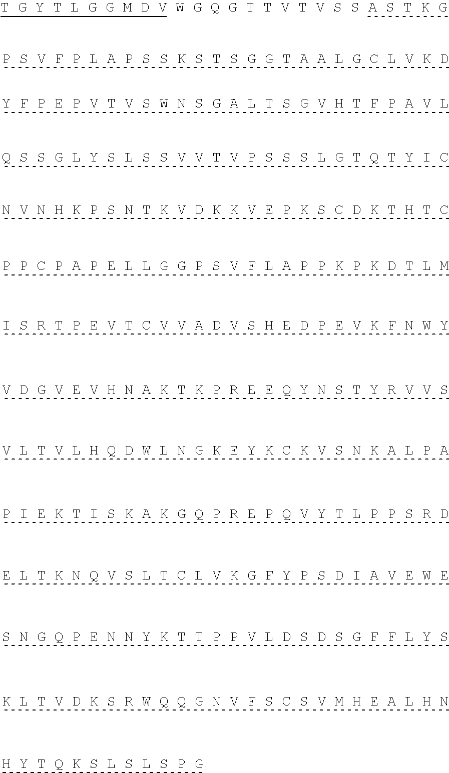

(SEQ ID NO: 41)
CDR-H1: G Y T F T D Y Y M H
(SEQ ID NO: 60)
CDR-H2: R I N P N S G G T N Y A Q K F Q G
(SEQ ID NO: 79)
CDR-H3: E G Y D F L T G Y T L G G M D V

The 6A11 light chain immunoglobulin comprises the amino acid sequence:

(SEQ ID NO: 22)
D I R L T Q S P S S L S A S V G D R V T V A C R A S
Q N I S S Y L N W Y Q Q K P G K A P K L L I Y A A S
S L Q S G V P S R F S G S G S G T D F T L T I S S L
Q P E D F A T Y Y C Q Q S Y S T P F T F G P G T K V

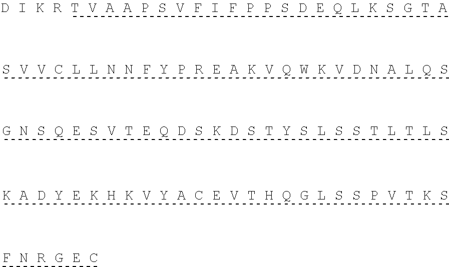

(SEQ ID NO: 98)
CDR-L1: R A S Q N I S S Y L N
(SEQ ID NO: 117)
CDR-L2: A A S S L Q S
(SEQ ID NO: 136)
CDR-L3: Q Q S Y S T P F T

The 5B7 heavy chain immunoglobulin comprises the amino acid sequence:

(SEQ ID NO: 4)
Q V Q L V Q S G A E V K K P G A S V K V S C K A S G
G T F S S Y A I S W V R Q A P G Q G L E W M G G I I
P I F G T A N Y A Q K F Q G R V T I T A D K S T S T
A Y M E L S S L R S E D T A V Y Y C A R V D Y D I L

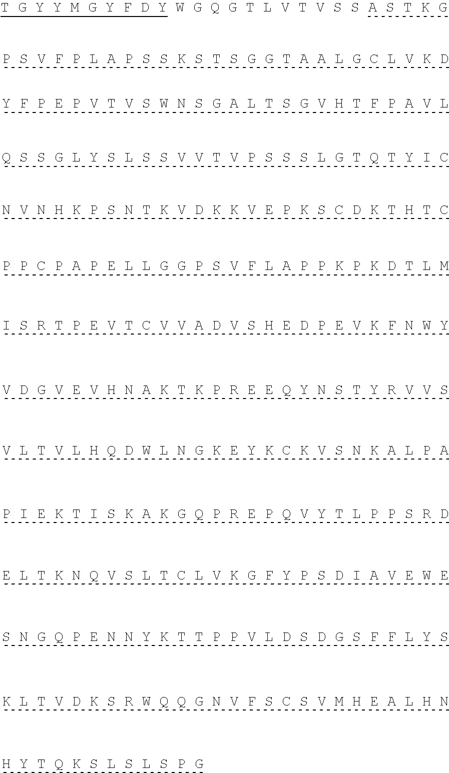

(SEQ ID NO: 42)
CDR-H1: G G T F S S Y A I S
(SEQ ID NO: 61)
CDR-H2: G I I P I F G T A N Y A Q K F Q G
(SEQ ID NO: 80)
CDR-H3: V D Y D I L T G Y Y M G Y F D Y

The 5B7 light chain immunoglobulin comprises the amino acid sequence:

(SEQ ID NO: 23)
D I V M T Q S P A T L S L S P G E R A T L S C R A
S Q S V S S Y L A W Y Q Q K P G Q A P R L L I Y D
A S N R A T G I P A R F S G S G S G T D F T L T I
S S L E P E D F A V Y Y C Q Q R S N W P I T F G Q

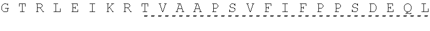

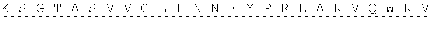

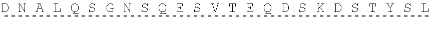

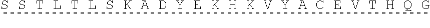

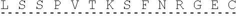

CDR-L1:
(SEQ ID NO: 99)
R A S Q S V S S Y L A
CDR-L2:
(SEQ ID NO: 118)
D A S N R A T
CDR-L3:
(SEQ ID NO: 137)
Q Q R S N W P I T

The 1B7 heavy chain immunoglobulin comprises the amino acid sequence:

(SEQ ID NO: 5)
Q V Q L V Q S G A K V K K P G A S A K V S C K A S
G Y T F T S Y A M H W V R Q A P G Q R L E W M G W
I N A G N G N T K Y S Q K F Q G R V T I T R D T S
A S T A Y M E L S S L R S E D T A V Y Y C A R A Y
Y D I L T G Y S V Y G M D V W G Q G T T V T V S S

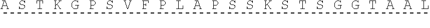

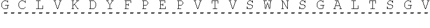

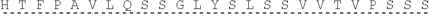

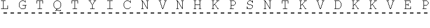

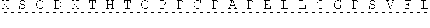

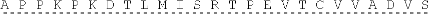

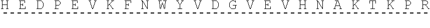

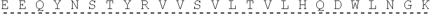

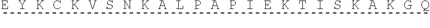

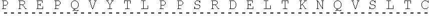

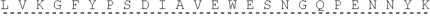

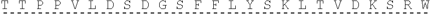

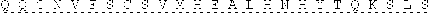

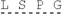

CDR-H1:
(SEQ ID NO: 43)
G Y T F T S Y A M H
CDR-H2:
(SEQ ID NO: 62)
W I N A G N G N T K Y S Q K F Q G
CDR-H3
(SEQ ID NO: 81)
A Y Y D I L T G Y S V Y G M D V

The 1B7 light vchain immunoglobulin comprises the amino acid sequence:

(SEQ ID NO: 24)
E I V L T Q S P A T L S L S P G E R A T L S C W T
S Q G V S G H L A W Y Q H K R G Q A P R L L I Y D
T S N R A T G V P A R F S G S G S G T D F T L T I
S R L E P E D F A V Y Y C Q Q Y G S S F W T F G Q

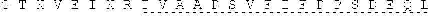

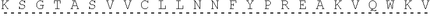

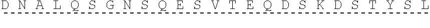

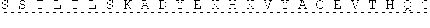

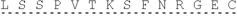

CDR-L1:
(SEQ ID NO: 100)
W T S Q G V S G H L A
CDR-L2:
(SEQ ID NO: 119)
D T S N R A T
CDR-L3:
(SEQ ID NO: 138)
Q Q Y G S S F W T

The 5E12 heavy chain immunoglobulin comprises the amino acid sequence:

(SEQ ID NO: 6)
Q V Q L V Q S G A E V K K P G S S V K V S C K A S
G G T F S S Y A I S W V R Q A P G Q G L E W M G W
M N P N S G N T G Y A Q K F Q G R V T M T R N T S
I S T A Y M E L S S L R S E D T A V Y Y C A R G H

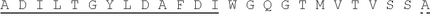

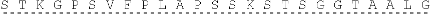

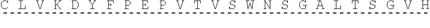

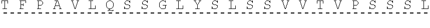

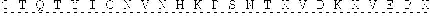

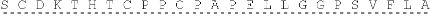

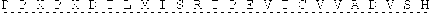

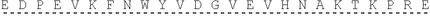

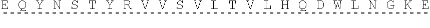

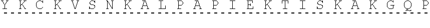

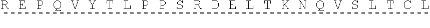

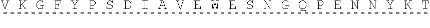

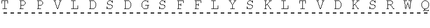

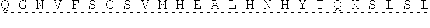

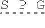

CDR-H1:
(SEQ ID NO: 44)
G G T F S S Y A I S
CDR-H2:
(SEQ ID NO: 63)
W M N P N S G N T G Y A Q K F Q G
CDR-H3:
(SEQ ID NO: 82)
G H A D I L T G Y L D A F D I

The 5E12 light chain immunoglobulin comprises the amino acid sequence:

(SEQ ID NO: 25)
E I V L T Q S P A T L S S S P G Q R A T L S C R A S
Q S I S S Y L A W Y V Q K P G Q A P S L L I Y D A S
N R A T G I P A R F S G S G S G T D F T L T I S S L
E P E D F A V Y Y C Q Q R S N W P I T F G Q G T R L

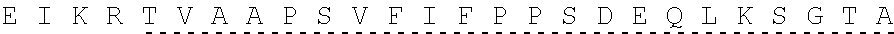

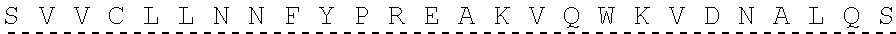

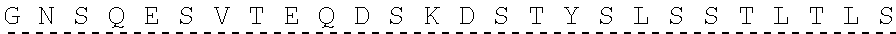

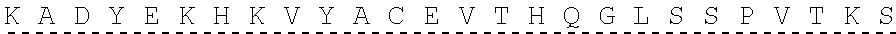

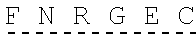

(SEQ ID NO: 101)
CDR-L1: R A S Q S I S S Y L A
(SEQ ID NO: 120)
CDR-L2: D A S N R A T
(SEQ ID NO: 139)
CDR-L3: Q Q R S N W P I T

The 5G10 heavy chain immunoglobulin comprises the amino acid sequence:

(SEQ ID NO: 7)
Q M Q L V Q S G A E V K K P G A S V K V S C K A S G
Y T F T S Y A M H W V R Q A P G Q R L E W M G W I N
A G N G N T K Y S Q K F Q G R V T I T R D T S A S T
A Y M E L S S L R S E D T A V Y Y C A R E M A V A G

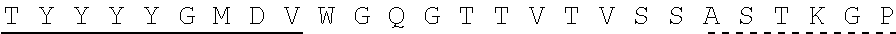

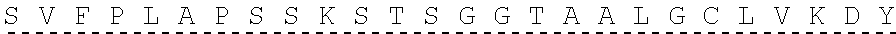

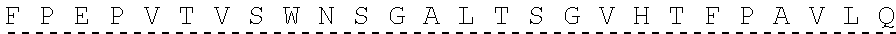

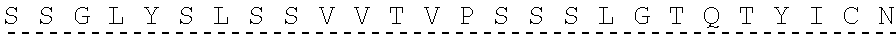

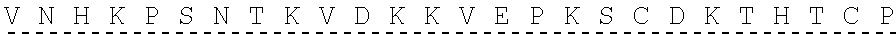

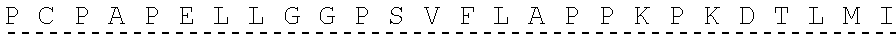

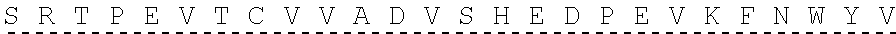

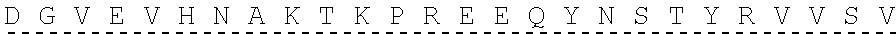

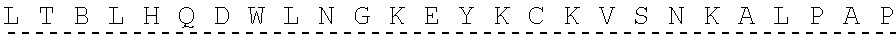

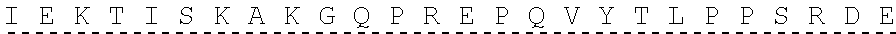

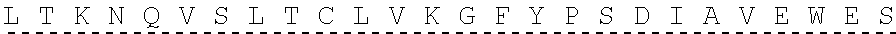

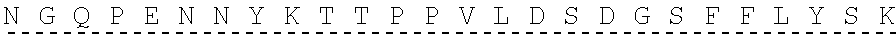

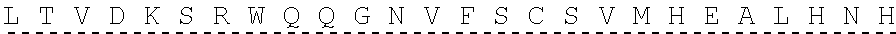

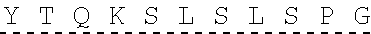

(SEQ ID NO: 45)
CDR-H1: G Y T F T S Y A M H
(SEQ ID NO: 64)
CDR-H2: W I N A G N G N T K Y S Q K F Q G
(SEQ ID NO: 83)
CDR-H3: E M A V A G T Y Y Y Y G M D V

The 5G10 light chain immunoglobulin comprises the amino acid sequence:

(SEQ ID NO: 26)
D I Q L T Q S P S T L S A S V G D R V T I T C R A S
Q S I S G W L A W Y Q Q K P G R A P K L L I Y K A S
S L E S G V P S R F S G S G S G T D F T L T I S S L
Q P E D I A T Y Y C Q Q F D N L L L M Y T F G Q G T

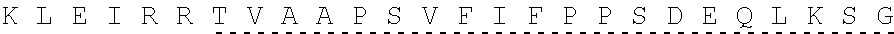

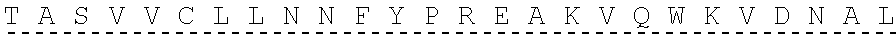

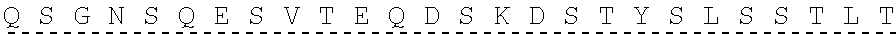

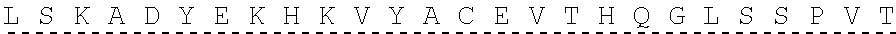

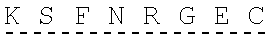

(SEQ ID NO: 102)
CDR-L1: R A S Q S I S G W L A
(SEQ ID NO: 121)
CDR-L2: K A S S L E S
(SEQ ID NO: 140)
CDR-L3: Q Q F D N L L L M Y T

The 8D12 heavy chain immunoglobulin comprises the amino acid sequence:

(SEQ ID NO: 8)
Q V Q L V Q S G A E V K K P G A S V K V S C K A S G
Y T F T S Y A M H W V R Q A P G Q R L E W M G W I N
A G N G N T K Y S Q K F Q G R V T I T R D T S A S T
A Y M E L S S L R S E D T A V Y Y C A R A Y Y D I L

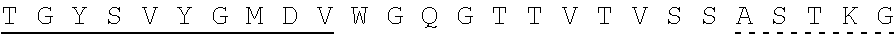

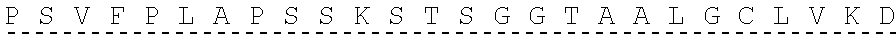

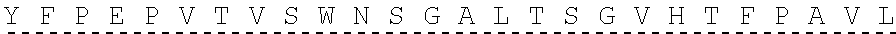

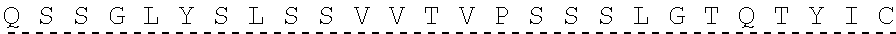

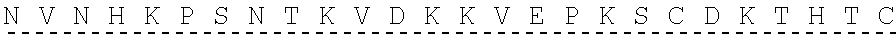

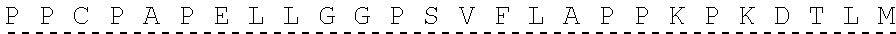

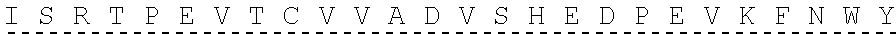

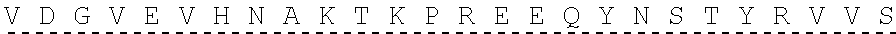

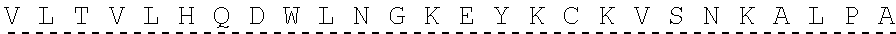

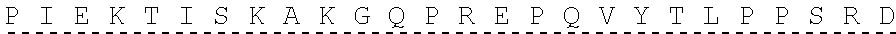

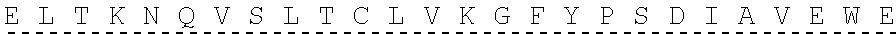

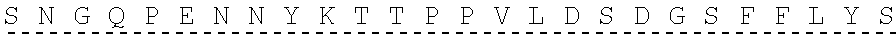

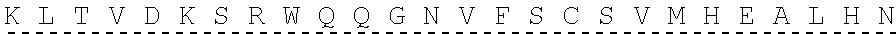

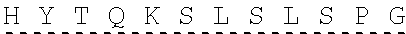

(SEQ ID NO: 46)
CDR-H1: G Y T F T S Y A M H
(SEQ ID NO: 65)
CDR-H2: W I N A G N G N T K Y S Q K F Q G
(SEQ ID NO: 84)
CDR-H3: A Y Y D I L T G Y S V Y G M D V

The 8D12 light chain immunoglobulin comprises the amino acid sequence:

(SEQ ID NO: 27)
E I V L T Q S P A T L S L S P G E R A T L S C R A N
E S V G N Y L A W Y Q Q K P G Q A P R L L I Y G A S
R R A T G I P D R F S G S G S G T D F T L T I S R L
E P E D F A V Y Y C Q Q Y G S S L L T F G G G T K V

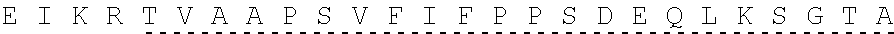

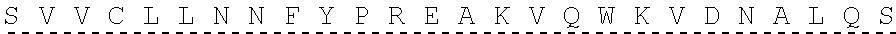

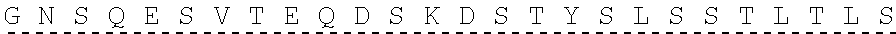

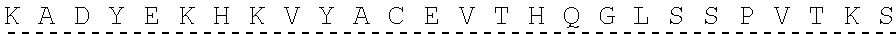

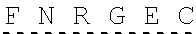

(SEQ ID NO: 103)
CDR-L1: R A N E S V G N Y L A
(SEQ ID NO: 122)
CDR-L2: G A S R R A T
(SEQ ID NO: 141)
CDR-L3: Q Q Y G S S L L T

The 2C5 heavy chain immunoglobulin comprises the amino acid sequence:

(SEQ ID NO: 9)
Q V Q L V Q S G A E V K K P G A S V K V S C K A S G
Y T F T G Y Y I H W V R Q A P G Q G L E W M G W I N
P S S G G T N Y A Q K F Q G R V T M T R D T S A S T
A F M S L S S L R S E D T A V Y Y C A R S Y Y D I L

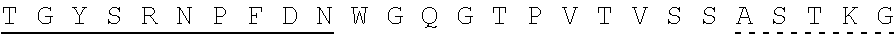

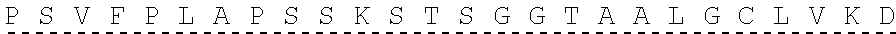

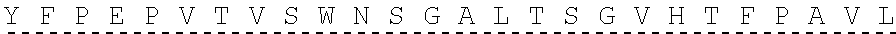

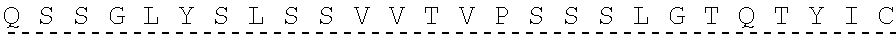

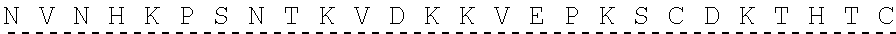

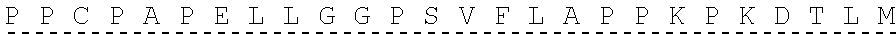

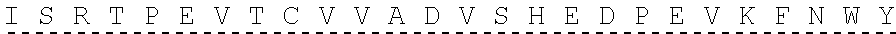

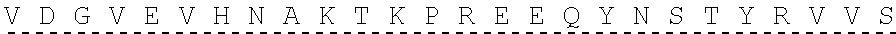

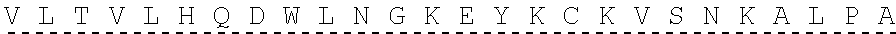

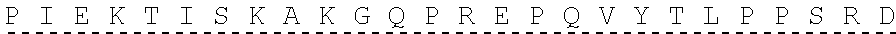

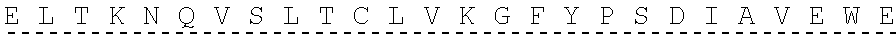

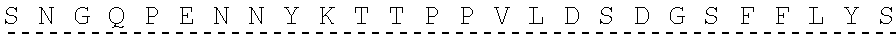

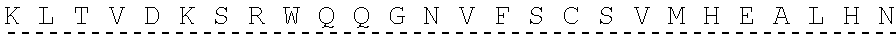

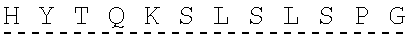

(SEQ ID NO: 47)
CDR-H1: G Y T F T G Y Y I H
(SEQ ID NO: 66)
CDR-H2: W I N P S S G G T N Y A Q K F Q G
(SEQ ID NO: 85)
CDR-H3: S Y Y D I L T G Y S R N P F D N

The 2C5 light chain immunoglobulin comprises the amino acid sequence:

(SEQ ID NO: 28)
E V V L T Q S P D S L A V S L G E R A T I N C K S S
Q S V L S S S N N K N Y L A W Y Q Q K P G Q P P K L
L I Y W A S T R E S G V P D R F S G S G S G T D F T
L T I S S L Q A E D V A V Y Y C Q Q Y Y S T P P T F

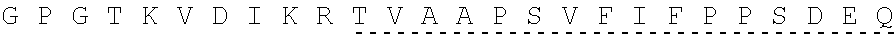

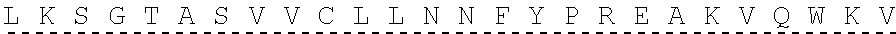

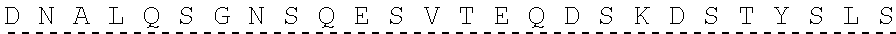

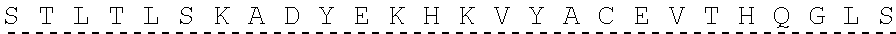

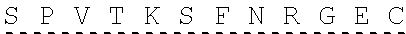

(SEQ ID NO: 104)
CDR-L1: K S S Q S V L S S S N N K N Y L A
(SEQ ID NO: 123)
CDR-L2: W A S T R E S
(SEQ ID NO: 142)
CDR-L3: Q Q Y Y S T P P T

The 6F5 heavy chain immunoglobulin comprises the amino acid sequence:

(SEQ ID NO: 10)
E V Q L V Q S G A E V K K P G S S V K V S C K A S G
G T F S S Y A I S W V R Q A P G Q G L E W M G G I S
A Y K G N T N Y A Q K L Q G R V T M T T D T S T S T
A Y M E L R S L R S E D T A V Y Y C A R G R L A E Y
D I L T G Y Y F N T W A F D I W G Q G T M V T V S S

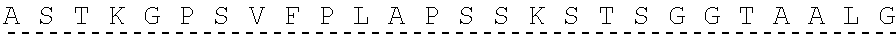

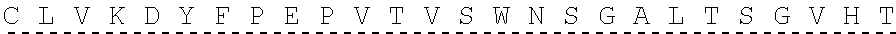

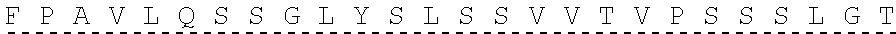

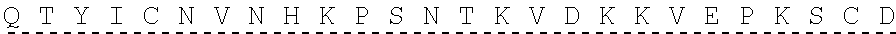

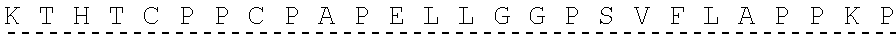

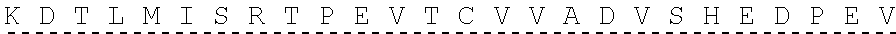

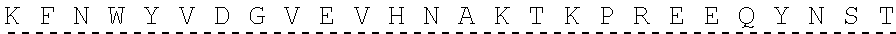

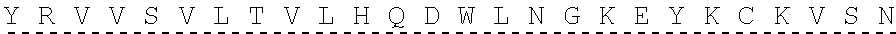

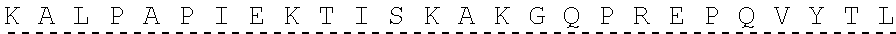

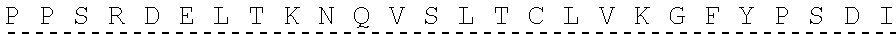

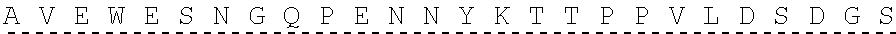

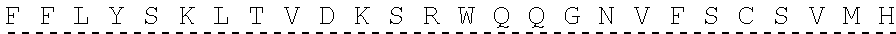

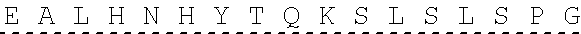

(SEQ ID NO: 48)
CDR-H1: G G T F S S Y A I S
(SEQ ID NO: 67)
CDR-H2: G I S A Y K G N T N Y A Q K L Q G
(SEQ ID NO: 86)
CDR-H3: G R L A E Y D I L T G Y Y F N T W A F D I

The 6F5 light chain immunoglobulin comprises the amino acid sequence:

(SEQ ID NO: 29)
Q S A L T Q P A S V S G S P G Q S I T I S C T G T S
S D V G G Y N Y V S W Y Q Q H P G K A P K L M I Y D
V S K R P S G V S N R F S G S K S G N T A S L T I S
G L Q A E D E A D Y Y C S S Y T S S S T P V L F G G

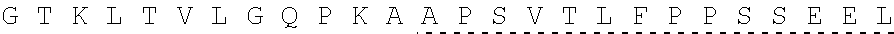

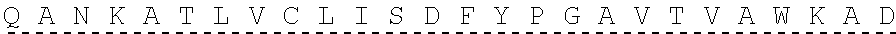

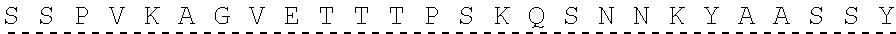

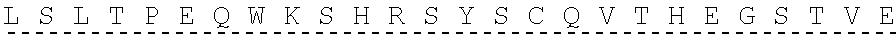

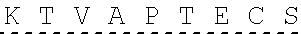

(SEQ ID NO: 105)
CDR-L1: T G T S S D V G G Y N Y V S
(SEQ ID NO: 124)
CDR-L2: D V S K R P S
(SEQ ID NO: 143)
CDR-L3: S S Y T S S S T P V L

The 8A12 heavy chain immunoglobulin comprises the amino acid sequence:

(SEQ ID NO: 11)
Q V Q L V Q S G G G L V Q P G G S L R L S C A A S G
F T F S S Y A M S W V R Q A P G K G L E W V S A I S
G S G G S T Y Y A D S V K G R F T I S R D N S K N T
L Y L Q M N S L R A E D T A V Y Y C A R E G Q G Y D
I L T G Y Y T R G Y Y F D Y W G Q G T L V T V S S A

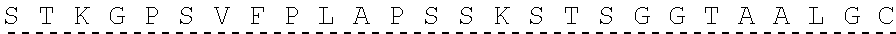

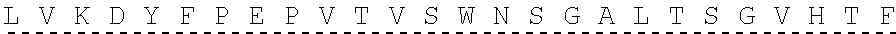

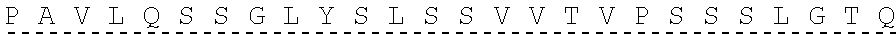

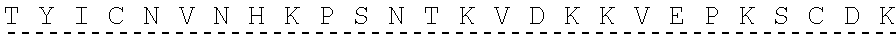

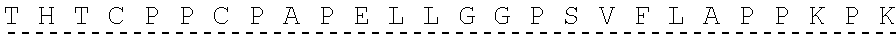

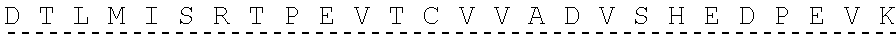

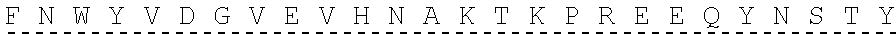

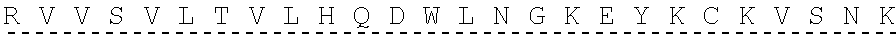

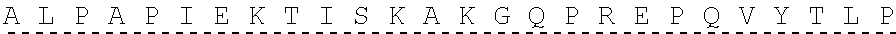

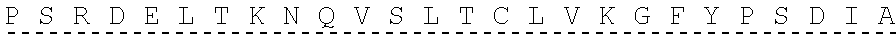

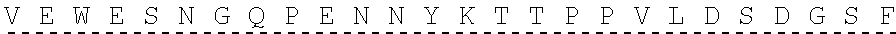

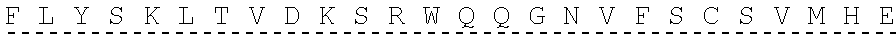

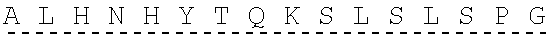

(SEQ ID NO: 49)
CDR-H1: G F T F S S Y A M S
(SEQ ID NO: 68)
CDR-H2: A I S G S G G S T Y Y A D S V K G
(SEQ ID NO: 87)
CDR-H3: E G Q G Y D I L T G Y Y T R G Y Y F D Y

The 8A12 light chain immunoglobulin comprises the amino acid sequence:

(SEQ ID NO: 30)
Q S A L T Q P A S V S G S P G Q S I T I S C T G T S
S D V G G Y N Y V S W Y Q Q L P G K A P K L M I Y E
V S K R P S G V P D R F S G S K S G N T A S L T I S
G L Q A E D E A D Y Y C S S Y A G S S S W V F G G G

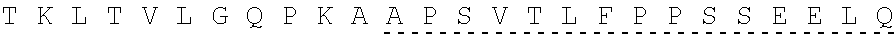

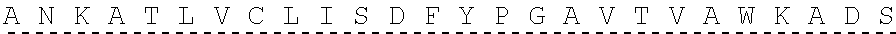

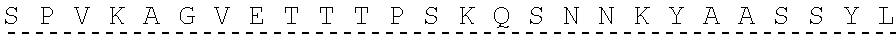

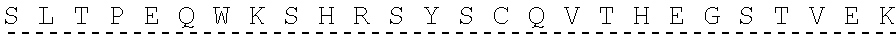

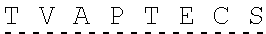

(SEQ ID NO: 106)
CDR-L1: T G T S S D V G G Y N Y V S
(SEQ ID NO: 125)
CDR-L2: E V S K R P S
(SEQ ID NO: 144)
CDR-L3: S S Y A G S S S W V

The 1B5 heavy chain immunoglobulin comprises the amino acid sequence:

(SEQ ID NO: 12)
Q V Q L Q E S G P G L V E P S E T L S L T C T V S G
G S I S G R N Y Y W G W I R Q S T G K G L E W I G S
I Y Y S G S T Y Y N P S L K S R V T I S V D T S K N
Q F S L K L S S V T A A D T A V Y Y C A R D L A N Y
D I L T G Y Y R A R G A F D I W G Q G T M V T V S S

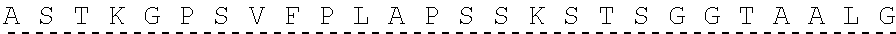

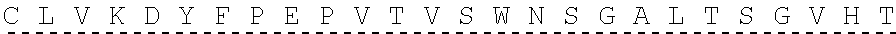

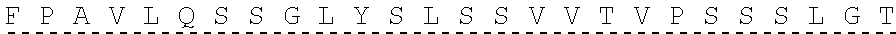

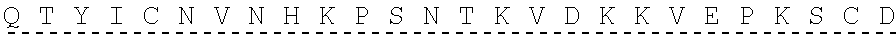

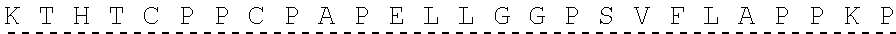

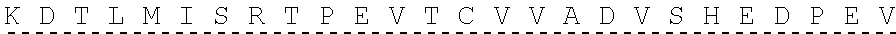

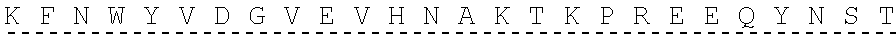

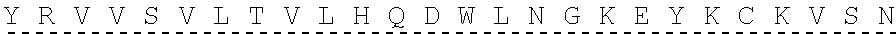

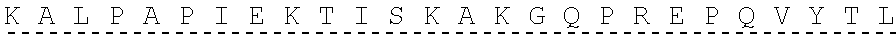

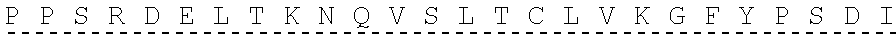

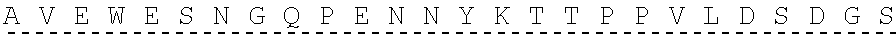

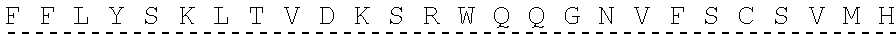

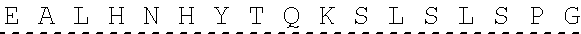

(SEQ ID NO: 50)
CDR-H1: G G S I S G R N Y Y
(SEQ ID NO: 69)
CDR-H2: S I Y Y S G S T Y Y N P S L K S
(SEQ ID NO: 88)
CDR-H3: D L A N Y D I L T G Y Y R A R G A F D I

The 1B5 light chain immunoglobulin comprises the amino acid sequence:

(SEQ ID NO: 31)
Q S A L T Q P A S V S G S P G Q S I T I S C T G T S
S D V G G Y N Y V S W Y Q Q H P G K A P K L M I Y D
V N K R P S G V P D R F S G S K S A N T A S L T V S
G L Q A E D E A D Y Y C S S Y T A G G N W V F G G G

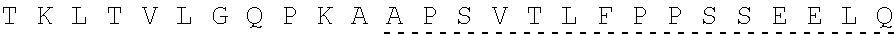

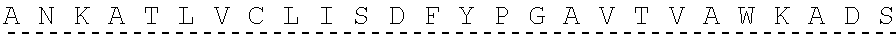

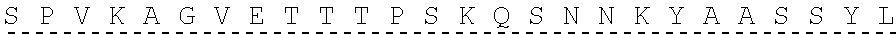

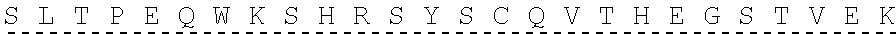

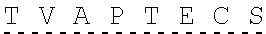

(SEQ ID NO: 107)
CDR-L1: T G T S S D V G G Y N Y V S
(SEQ ID NO: 126)
CDR-L2: D V N K R P S
(SEQ ID NO: 145)
CDR-L3: S S Y T A G G N W V

The 10B1 heavy chain immunoglobulin comprises the amino acid sequence:

(SEQ ID NO: 13)
E V Q L V E S G A E V K K P G A S V K V S C K A S G
Y T F T S Y G I S W V R Q A P G Q G L E W M G W I S
A Y N G N T N Y A Q K L Q G R V T M T T D T S T S T
A Y M E L R S L R S D D T A V Y Y C A R A D Y D I L

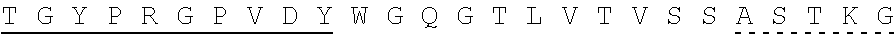

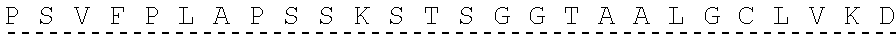

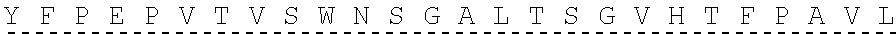

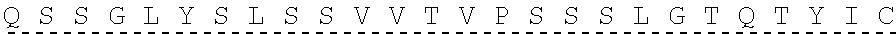

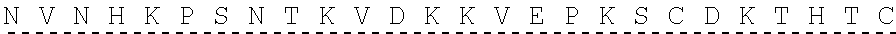

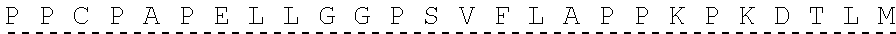

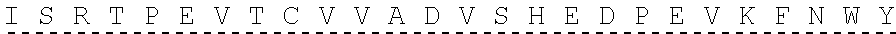

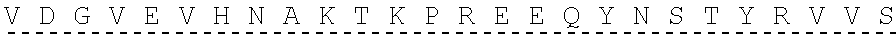

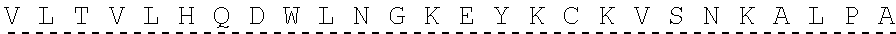

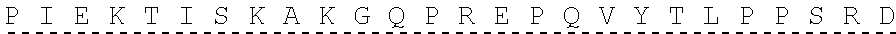

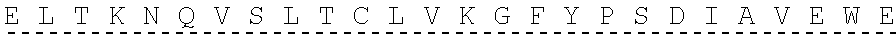

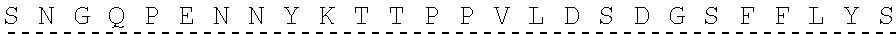

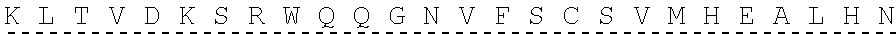

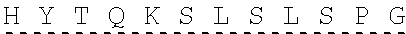

(SEQ ID NO: 51)
CDR-H1: G Y T F T S Y G I S
(SEQ ID NO: 70)
CDR-H2: W I S A Y N G N T N Y A Q K L Q G
(SEQ ID NO: 89)
CDR-H3: A D Y D I L T G Y P R G P V D Y

The 10B1 light chain immunoglobulin comprises the amino acid sequence:

(SEQ ID NO: 32)
Q S A L T Q P P S A S G S P G Q S V T I S C T G T S
S D V G H Y N Y V S W Y Q Q H P G K A P K L I I F D
V S D R P S G V S N R F S G S K S G N T A S L T I S
G L Q A E D E A N Y Y C S S Y T S R N T L I F G G G

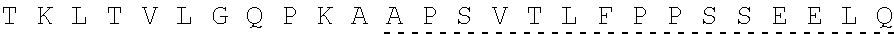

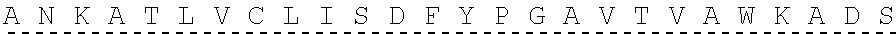

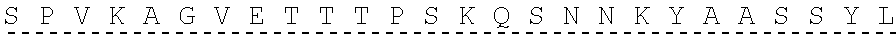

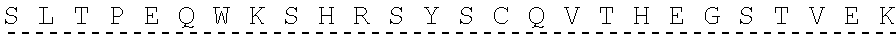

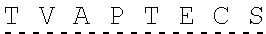

(SEQ ID NO: 108)
CDR-L1: T G T S S D V G H Y N Y V S
(SEQ ID NO: 127)
CDR-L2: D V S D R P S
(SEQ ID NO: 146)
CDR-L3: S S Y T S R N T L I

The 4A2 heavy chain immunoglobulin comprises the amino acid sequence:

(SEQ ID NO: 14)
Q V Q L V Q S G G G V V Q P G R S L R L S C A A S
G F T F S S Y G M H W V R Q A P G K G L E W V A V
I S H D G S N K Y Y A D S V K G R F T I S R D N S
K N T L Y L Q M N S L R A E D T A V Y Y C A K D R
S Y Y D I L T G Y Y I P Y Y Y G M D V W G Q G T T

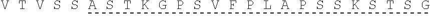

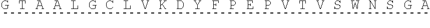

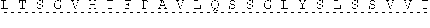

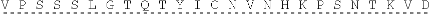

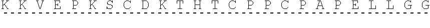

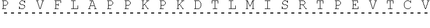

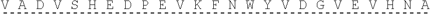

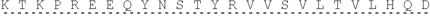

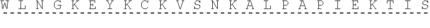

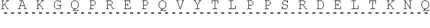

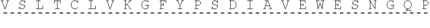

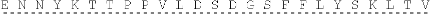

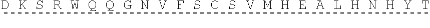

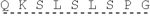

CDR-H1:
(SEQ ID NO: 52)
G F T F S S Y G M H
CDR-H2:
(SEQ ID NO: 71)
V I S H D G S N K Y Y A D S V K G
CDR-H3:
(SEQ ID NO: 90)
D R S Y Y D I L T G Y Y I P Y Y Y G M D V

The 4A2 light chain immunoglobulin comprises the amino acid sequence:

(SEQ ID NO: 33)
Q A V L T Q P P S A S G T P G Q K V T I S C S G S
S S D I G S N N V N W Y Q Q F P G T A P K L L I H
S N N L R P S G V P V R F S G S K S G T S A S L A
I S G L Q S E D E A D Y Y C S A W D D S V N G V A

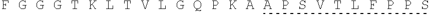

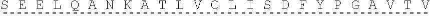

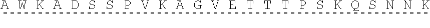

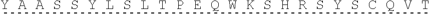

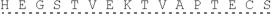

CDR-L1:
(SEQ ID NO: 109)
S G S S S D I G S N N V N
CDR-L2:
(SEQ ID NO: 128)
S N N L R P S
CDR-L3:
(SEQ ID NO: 147)
S A W D D S V N G V A

The 14F11 heavy chain immunoglobulin comprises the amino acid sequence:

(SEQ ID NO: 15)
Q V Q L V Q S G A E V K K P G S S V K V S C K A S
G G T F S S Y A I S W V R Q A P G Q G L E W M G R
I N P N S G G T N Y A Q K F Q G R V T M T R D T S
I S T A Y M E L S R L R S D D T F V Y Y C A R E G
Y D F L T G Y T L G G M D V W G Q G T T V T V S S

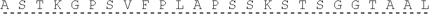

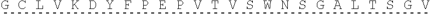

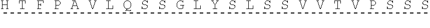

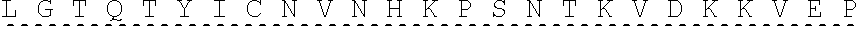

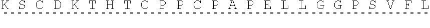

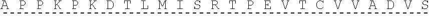

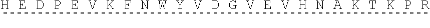

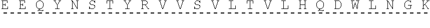

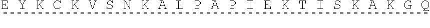

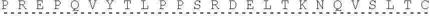

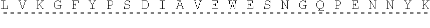

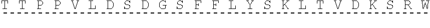

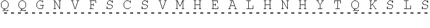

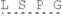

CDR-H1:
(SEQ ID NO: 53)
G G T F S S Y A I S
CDR-H2:
(SEQ ID NO: 72)
R I N P N S G G T N Y A Q K F Q G
CDR-H3:
(SEQ ID NO: 91)
E G Y D F L T G Y T L G G M D V

The 14F11 light chain immunoglobulin comprises the amino acid sequence:

(SEQ ID NO: 34)
Q S V L T Q P P S V S A A P G Q K V T I S C S G S
S S N I G N N Y V S W Y Q Q L P G T A P K L L I Y
D N N K R P S G I P D R F S A S K S G T S A S L A
I S G L Q A E D E A D Y Y C Q S Y D S S L S G V V

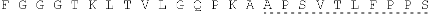

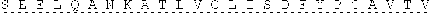

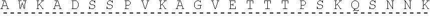

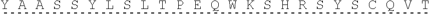

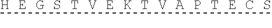

CDR-L1:
(SEQ ID NO: 110)
S G S S S N I G N N Y V S
CDR-L2:
(SEQ ID NO: 129)
D N N K R P S
CDR-L3:
(SEQ ID NO: 148)
Q S Y D S S L S G V V

The 9F8 heavy chain immunoglobulin comprises the amino acid sequence:

(SEQ ID NO: 16)
Q V Q L Q Q S G A E V K K P G A S V K V S C K A S
G Y T F T S Y Y M H W V R Q A P G Q G L E W M G W
I N A G N G N T K Y S Q K F Q G R V T I T R D T S
A S T A Y M E L S S L R S E D T A V Y Y C A R D N
L S Y D I L T G Y S F G G W F D P W G Q G T L V T

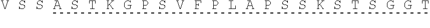

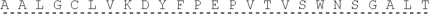

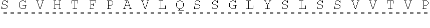

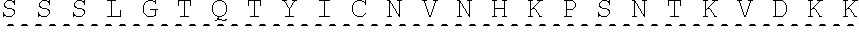

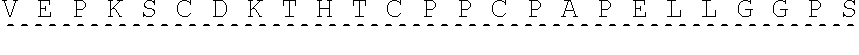

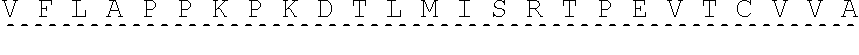

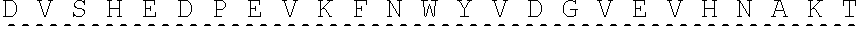

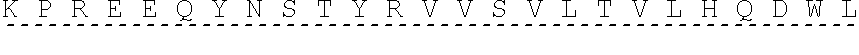

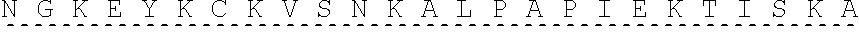

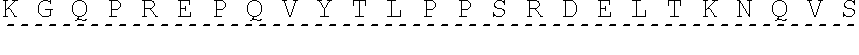

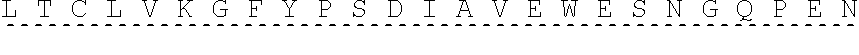

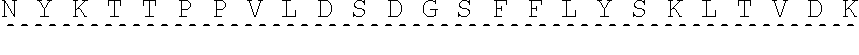

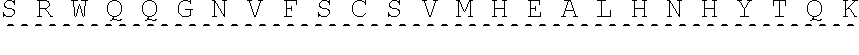

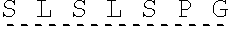

CDR-H1:
(SEQ ID NO: 54)
G Y T F T S Y Y M H
CDR-H2:
(SEQ ID NO: 73)
W I N A G N G N T K Y S Q K F Q G
CDR-H3:
(SEQ ID NO: 92)
D N L S Y D I L T G Y S F G G W F D P

The 9F8 light chain immunoglobulin comprises the amino acid sequence:

(SEQ ID NO: 35)
Q S A L T Q P A S V S G S P G Q S I T I S C T G T
S S D I G R Y N Y V S W Y Q Q R P G R A P K V I I
Y D V S S R P S G V S K R F S A S K S G N T A S L
T I S G L Q A E D E A V Y Y C S S Y T R I T T L Y

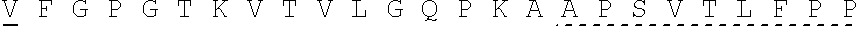

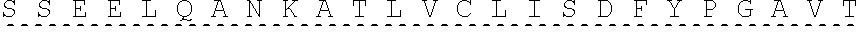

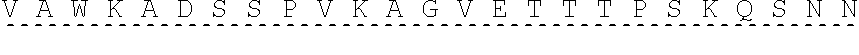

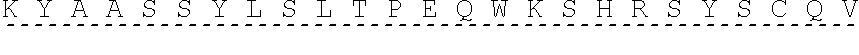

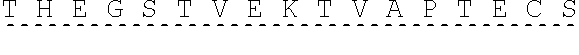

CDR-L1:
(SEQ ID NO: 111)
T G T S S D I G R Y N Y V S
CDR-L2:
(SEQ ID NO: 130)
D V S S R P S
CDR-L3:
(SEQ ID NO: 149)
S S Y T R I T T L Y V

The 5D10 heavy chain immunoglobulin comprises the amino acid sequence:

(SEQ ID NO: 17)
Q I Q L V Q S G P E V K K P G A S V K V S C K A S
G Y T F T G Y Y M H W V R Q A P G Q G L E W M G W
I N P N S G G T N Y A Q K F Q G R V T M T R D T S
I S T A Y M E L S R L R S D D T A V Y Y C A R G Y

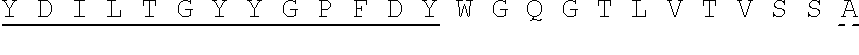

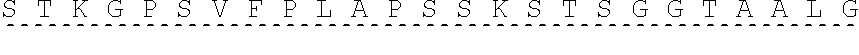

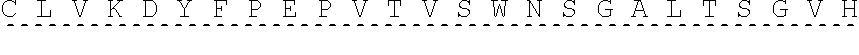

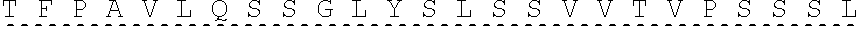

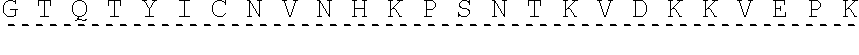

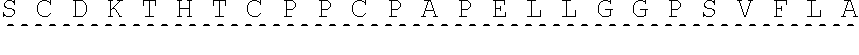

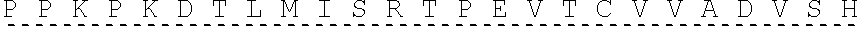

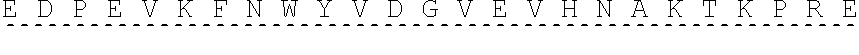

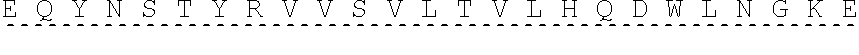

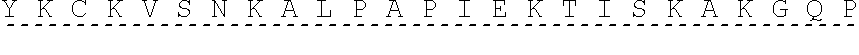

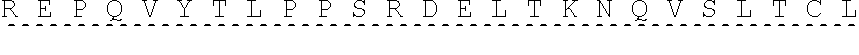

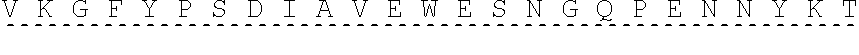

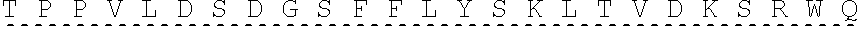

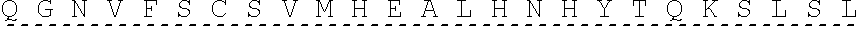

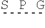

CDR-H1:
(SEQ ID NO: 55)
G Y T F T G Y Y M H
CDR-H2:
(SEQ ID NO: 74)
W I N P N S G G T N Y A Q K F Q G
CDR-H3:
(SEQ ID NO: 93)
G Y Y D I L T G Y Y G P F D Y

The 5D10 light chain immunoglobulin comprises the amino acid sequence:

(SEQ ID NO: 36)
Q P G L T Q P P S A S G T P G Q R V T I S C S G S
S S N I G N N T V N W Y Q Q L P G T A P R L L I Y
T N D Q R P S G V P D R F S G S K S G T S A S L A
I S G L Q S E D E A Q Y Y C S T W D D R Q K A V V

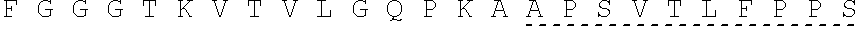

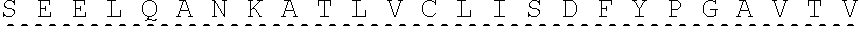

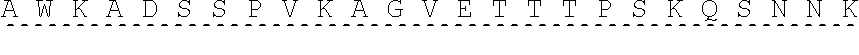

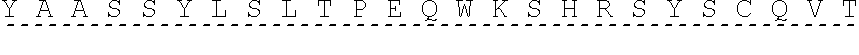

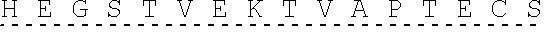

CDR-L1:
(SEQ ID NO: 112)
S G S S S N I G N N T V N
CDR-L2:
(SEQ ID NO: 131)
T N D Q R P S
CDR-L3:
(SEQ ID NO: 150)
S T W D D R Q K A V V

The 1D6 heavy chain immunoglobulin comprises the amino acid sequence:

(SEQ ID NO: 18)
Q V T L K E S G P T L V K P T Q T L T L T C T F S
G F S L S T D G V G V G W I R Q P P G K A L E W L
A L I Y G D D D K R Y S P S L K S R L T I T K D T
S K N Q V V L T M T N M D P V D T A T Y Y C A H S
I S N Y D I L T G H W I A K T Y D Y W G Q G T L V

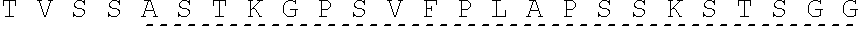

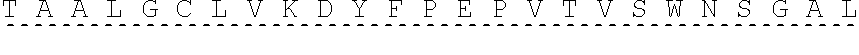

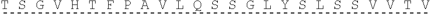

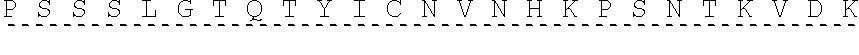

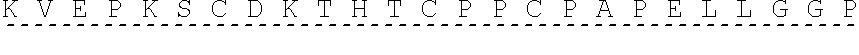

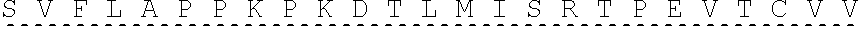

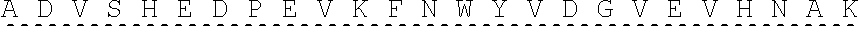

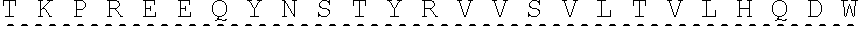

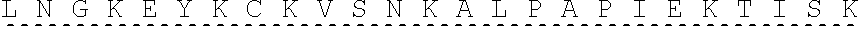

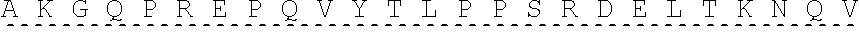

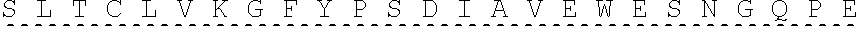

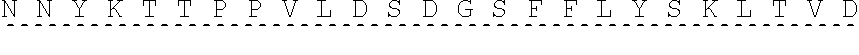

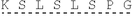

CDR-H1:
(SEQ ID NO: 56)
G F S L S T D G V G V G
CDR-H2:
(SEQ ID NO: 75)
L I Y G D D D K R Y S P S L K S
CDR-H3:
(SEQ ID NO: 94)
S I S N Y D I L T G H W I A K T Y D Y

The 1D6 light chain immunoglobulin comprises the amino acid sequence:

(SEQ ID NO: 37)
Q S A L T Q P P S A S G S P G Q S V S I S C I G S
R S D I G Y Y N Y V S W Y Q Q H P G E A P K L I I
F D V S K R P S G V P D R F S G S K S G N T A S L
T V S G L Q P E D E A D Y Y C A S Y G G R N N L L

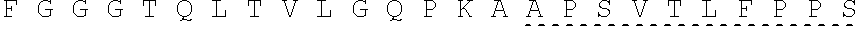

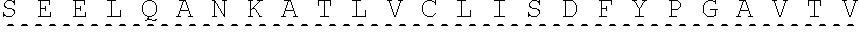

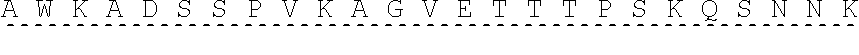

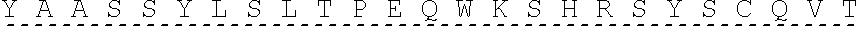

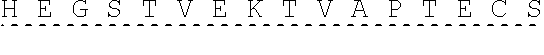

CDR-L1:
(SEQ ID NO: 113)
I G S R S D I G Y Y N Y V S
CDR-L2:
(SEQ ID NO: 132)
D V S K R P S
CDR-L3:
(SEQ ID NO: 151)
A S Y G G R N N L L

The 1F4 heavy chain immunoglobulin comprises the amino acid sequence:

(SEQ ID NO: 19)
Q V T L K E S G P T L V K P T Q T L T L T C T F S G
F S L S T S R V G V G W I R Q P P G K A L E W L A R
I D W D D D K Y Y S T S L K T R L T I S K D T S K N
Q V V L T M T N M D P V D T A T Y Y C A R T H T Y Y
D I L T G Y S Y H V Y F D Y W G Q G T L V T V S S A

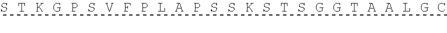

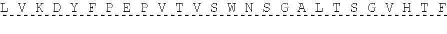

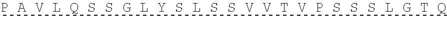

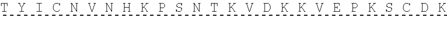

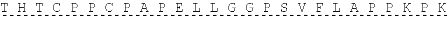

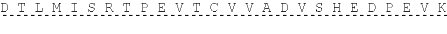

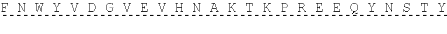

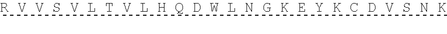

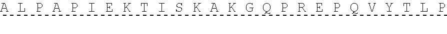

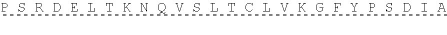

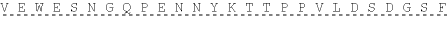

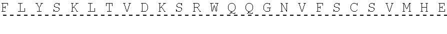

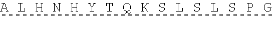

(SEQ ID NO: 57)
CDR-H1: G F S L S T S R V G V G
(SEQ ID NO: 76)
CDR-H2: R I D W D D D K Y Y S T S L K T
(SEQ ID NO: 95)
CDR-H3: T H T Y Y D I L T G Y S Y H V Y F D Y

The 1F4 light chain immunoglobulin comprises the amino acid sequence:

(SEQ ID NO: 38)
Q S A L T Q P P S A S G S R G Q S V S I S C S G S R
S D I G Y Y N Y V S W Y Q Q H P G K A P K L I I F D
V N K R P S G V P D R F S G S K S G N T A S L T V S
G L Q P E D E A D Y Y C A S Y G G R N N L L F G G G

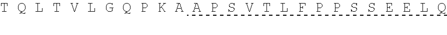

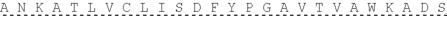

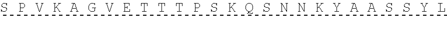

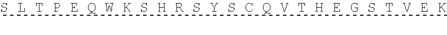

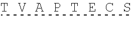

(SEQ ID NO: 114)
CDR-L1: S G S R S D I G Y Y N Y V S
(SEQ ID NO: 133)
CDR-L2: D V N K R P S
(SEQ ID NO: 152)
CDR-L3: A S Y G G R N N L L

In an embodiment of the invention, a polynucleotide encoding a 5B7 light chain immunoglobulin variable domain comprises the nucleotide sequence:

(SEQ ID NO: 162)

gacatcgtcatgacccagtccccagccaccttgtccttgtccccaggaga
gagagccaccttgagttgcagggcctcccaatccgtctcctcctacttgg
cctggtatcagcagaagcctggtcaggccccaagattgttgatctacgac
gcctccaaccgtgccaccggtatcccagccagattctccggttctggttc
cgggacggacttcaccttgaccatctcctccttggagcctgaggacttcg
ccgtctactactgccaacagagatccaactggcctatcaccttcggtcag
gggacgcgtttggagatcaagcgtaccgtcgccgccccatccgtcttcat
cttcccaccttccgatgagcagttgaagtccggtaccgcttccgttgttt
gtttgttgaacaacttctacccacgtgaggctaaggttcagtggaaggtt
gacaacgctttgcaatccggtaactcccaagaatccgttactgagcagga
ttctaaggattccacttactcattgtcctccactttgactttgtccaagg
ctgattacgagaagcacaaggtttacgcttgcgaggttacacatcagggt
ttgtcctccccagttactaagtccttcaacagaggagagtgt

In an embodiment of the invention, a polynucleotide encoding a 5B7 heavy chain immunoglobulin variable domain comprises the nucleotide sequence:

(SEQ ID NO: 163)

caggttcagttggtccagtccggtgccgaggtcaagaagcctggtgcctc
cgtcaaggtctcttgcaaagcctccggaggaaccttctcctcctacgcca
tctcctgggtcagacaagccccaggtcaaggtttggagtggatgggtgga
atcatcccaatcttcggaaccgccaactacgcccagaagttccagggtag
agtcaccatcaccgctgacaagtccacctccaccgcctacatggagttgt
cctccttgcgttccgaggacactgccgtctactactgcgcccgtgtcgac
tacgacatcttgaccggttactacatgggttacttcgactactggggtca
gggaaccttggtcaccgtctcgagtgcttctactaagggaccatccgttt
ttccattggctccatcctctaagtctacttccggtggaaccgctgctttg
ggatgtttggttaaagactacttcccagagccagttactgtttcttggaa
ctccggtgctttgacttctggtgttcacactttcccagctgttttgcaat
cttccggtttgtactctttgtcctccgttgttactgttccatcctcttcc
ttgggtactcagacttacatctgtaacgttaaccacaagccatccaacac
taaggttgacaagaaggttgagccaaagtcctgtgacaagactcatactt
gtccaccatgtccagctccagaattgttgggtggtccttccgtttttttg
gccccaccaaagccaaaggacactttgatgatctccagaactccagaggt
tacatgtgttgttgctgacgtttctcacgaggacccagaggttaagttca
actggtacgttgacggtgttgaagttcacaacgctaagactaagccaaga
gaggagcagtacaactccacttacagagttgtttccgttttgactgtttt
gcaccaggattggttgaacggaaaggagtacaagtgtaaggtttccaaca
aggctttgccagctccaatcgaaaagactatctccaaggctaagggtcaa
ccaagagagccacaggtttacactttgccaccatccagagatgagttaac
taagaaccaggtttccttgacttgtttggttaagggattctacccatccg
acattgctgttgaatgggagtctaacggtcaaccagagaacaactacaag
actactccacctgttttggactctgacggttcctttttcttgtactccaa
gttgactgttgacaagtccagatggcaacagggtaacgttttctcctgtt
ccgttatgcatgaggctttgcacaaccactacactcaaaagtccttgtct
ttgtcccctggt

The scope of the present invention includes anti-BLyS antibodies and antigen-binding fragments thereof and polypeptides wherein the light chain (e.g., 6C10, 10C11, 6A11, 5B7, 1B7, 5E12, 5G10, 8D12, 2C5, 6F5, 8A12, 1B5, 10B1, 4A2, 14F11, 9F8, 5D10, 1D6 or 1F4) and/or heavy chain (e.g., 6C10, 10C11, 6A11, 5B7, 1B7, 5E12, 5G10, 8D12, 2C5, 6F5, 8A12, 1B5, 10B1, 4A2, 14F11, 9F8, 5D10, 1D6 or 1F4) immunoglobulin variable region is linked to any immunoglobulin constant domain. In an embodiment of the invention, the light chain variable region is linked to a kappa or lambda chain constant region. In an embodiment of the invention, the heavy chain variable region is linked to a IgA (e.g., IgA-1 or IgA-2), IgD, IgE, IgM or IgG, e.g., gamma-1, gamma-2, gamma-3 or gamma-4 chain constant region.

Antibody or antigen-binding fragment thereof CDRs may be as identified by any of the methods set forth in Chothia et al., J. Mol. Biol. 186:651-663 (1985); Novotny and Haber, Proc. Natl. Acad. Sci. USA 82:4592-4596 (1985) or Kabat, E. A. et al., Sequences of Proteins of Immunological Interest, National Institutes of Health, Bethesda, Md., (1987)).

The present invention includes anti-BLyS “monoclonal antibodies,”; e.g., which are obtained from a population of substantially homogeneous antibodies, i.e., the individual antibodies comprising the population are identical except for possible naturally occurring mutations that may be present in minor amounts. Monoclonal antibodies to be used in accordance with the present invention may be made by the hybridoma method first described by Kohler, et al., (1975) Nature 256: 495.

The present invention includes anti-BLyS bispecific or bifunctional antibodies and antigen-binding fragments thereof. Such antibodies and fragments are hybrids having two different heavy/light chain pairs and two different binding sites. Bispecific antibodies can be produced by a variety of methods including fusion of hybridomas or linking of Fab′ fragments. See, e.g., Songsivilai, et al., (1990) Clin. Exp. Immunol. 79: 315-321, Kostelny, et al., (1992) J Immunol. 148:1547- 1553. In addition, bispecific antibodies may be formed as “diabodies” (Holliger, et al., (1993) PNAS USA 90:6444-6448) or as “Janusins” (Traunecker, et al., (1991) EMBO J. 10:3655-3659 and Traunecker, et al., (1992) Int. J. Cancer Suppl. 7:51-52).

The present invention includes anti-BLyS fully human antibodies and antigen-binding fragments thereof. Such antibodies and fragments include human immunoglobulin protein sequences only. A fully human antibody may contain non-human, e.g., murine, carbohydrate chains if produced in a mouse, in a mouse cell or in a hybridoma derived from a mouse cell. Similarly, an anti-BLyS mouse antibody or antigen-binding fragment thereof refers to an antibody or fragment which comprises mouse immunoglobulin protein sequences only. A fully human anti-BLyS antibody or antigen-binding fragment thereof of the present invention is not an antibody or fragment that is identical to any antibody or fragment that is or has been generated naturally in the body of a human.

The present invention includes anti-BLyS recombinant antibodies and antigen-binding fragments thereof. Recombinant antibodies and fragments of the present invention are produced using recombinant DNA technology wherein DNA encoding an immunoglobulin polypeptide chain is expressed in a host cell (e.g., bacterial, mammalian (e.g., Chinese hamster ovary) or fungal (e.g, Pichia)) which is cultured under conditions favorable to expression of the immunoglobulin polypeptide. Methods for making recombinant antibodies and antigen-binding fragments have been described, e.g., by Boss et al. (U.S. Pat. No. 4,816,397), Cabilly et al. (U.S. Pat. No. 4,816,567), Law et al. (European Patent Application Publication No. 438 310) and Winter (European Patent Application Publication No. 239400). A recombinant antibody or antigen-binding fragment of the invention may be, for example, recombinant and fully human. A recombinant antibody or antigen-binding fragment of the invention may be, for example, monoclonal, i.e., the recombinant antibody or fragment is obtained from a population of substantially homogeneous antibodies or fragments. In an embodiment of the invention, a recombinant antibody or antigen binding fragment is the product of a process including introducing a polynucleotide encoding one or more immunoglobulin chain polypeptides (e.g., heavy and light chain) into a host cell and culturing the host cell in a medium under conditions favorable to expression of the immunoglobulin polypeptide chains. For example, in an embodiment of the invention, the chains are isolated from the culture medium and/or host cells.

The present invention includes displayed anti-BLyS antibodies and antigen-binding fragments thereof which are located, in part or wholly, on the surface of a cell or phage or virus or virus-like particle.

The present invention includes anti-BLyS chimeric antibodies and antigen-binding fragments thereof. Such antibodies and fragments comprise an immunoglobulin variable region from one organism (e.g., human, mouse, horse, rabbit, dog, cow, chicken) fused or chimerized with a polypeptide, e.g., an immunoglobulin constant domain, from another organism (e.g., human, mouse, horse, rabbit, dog, cow, chicken).

The present invention includes anti-BLyS single-chain antibodies which comprise V_{H} and V_{L} domains of an antibody, wherein these domains are present in a single polypeptide chain. Generally, the sFv polypeptide further comprises a polypeptide linker between the V_{H} and V_{L} domains which enables the sFv to form the desired structure for antigen binding. Techniques described for the production of single chain antibodies (U.S. Pat. Nos. 5,476,786; 5,132,405 and 4,946,778) can be adapted to produce anti-IGF1R-specific single chain antibodies. For a review of sFv see Pluckthun in The Pharmacology of Monoclonal Antibodies, vol. 113, Rosenburg and Moore eds. Springer-Verlag, N.Y., pp. 269-315 (1994).

The present invention includes anti-BLyS disulfide stabilized Fv fragments (dsFv) which are immunoglobulins comprising a variable heavy chain (V_{H}) and a variable light chain (V_{L}) which are linked by a disulfide bridge.

Antigen-binding fragments of anti-BLyS antibodies include F(ab)₂ fragments which may be produced by enzymatic cleavage of an IgG by, for example, pepsin. Fab fragments may be produced by, for example, reduction of F(ab)₂ with dithiothreitol or mercaptoethylamine. A Fab fragment is a V_{L}-C_{L} chain appended to a V_{H}-C_{H1} chain by a disulfide bridge. A F(ab)₂ fragment is two Fab fragments which, in turn, are appended by two disulfide bridges. The Fab portion of an F(ab)₂ molecule includes a portion of the F_{c} region between which disulfide bridges are located.

An anti-BLyS F_{V} fragment is a V_{L} or V_{H} region comprising CDRs which bind specifically to BLys.

The anti-BLyS antibodies and antigen-binding fragments of the invention may also be conjugated to a chemical moiety. The chemical moiety may be, inter alia, a polymer, a radionuclide or a cytotoxic factor. In an embodiment of the invention, the chemical moiety is a polymer which increases the half-life of the antibody molecule in the body of a subject. Suitable polymers include, but are not limited to, polyethylene glycol (PEG) (e.g., PEG with a molecular weight of 2 kDa, 5 kDa, 10 kDa, 12 kDa, 20 kDa, 30 kDa or 40 kDa), dextran and monomethoxypolyethylene glycol (mPEG). Lee, et al., (1999) (Bioconj. Chem. 10:973-981) discloses PEG conjugated single-chain antibodies. Wen, et al., (2001) (Bioconj. Chem. 12:545-553) disclose conjugating antibodies with PEG which is attached to a radiometal chelator (diethylenetriaminpentaacetic acid (DTPA)).

The anti-BLyS antibodies and antibody fragments of the invention may also be conjugated with labels such as ⁹⁹Tc, ⁹⁰Y, ¹¹¹In, ³²P, ¹⁴C, ¹²⁵I, ³H, ¹³¹I, ¹¹C, ¹⁵O, ¹³N, ¹⁸F, ³⁵S, ⁵¹Cr, ⁵⁷To, ²²⁶Ra, ⁶⁰Co, ⁵⁹Fe, ⁵⁷Se, ¹⁵²Eu, ⁶⁷CJ, ²¹⁷Ci, ²¹¹At, ²¹²Pb, ⁴⁷Sc, ¹⁰⁹Pd, ²³⁴Th, and ⁴⁰K, ¹⁵⁷Gd, ⁵⁵Mn, ⁵²Tr and ⁵⁶Fe.

The anti-BLyS antibodies and antibody fragments of the invention may also be conjugated with fluorescent or chemilluminescent labels, including fluorophores such as rare earth chelates, fluorescein and its derivatives, rhodamine and its derivatives, isothiocyanate, phycoerythrin, phycocyanin, allophycocyanin, o-phthaladehyde, fluorescamine, ¹⁵²Eu, dansyl, umbelliferone, luciferin, luminal label, isoluminal label, an aromatic acridinium ester label, an imidazole label, an acridimium salt label, an oxalate ester label, an aequorin label, 2,3-dihydrophthalazinediones, biotin/avidin, spin labels and stable free radicals.

Antibodies and antigen-binding fragments thereof of this invention can be conjugated to a therapeutic agent to form an immunoconjugate such as an antibody-drug conjugate (ADC). Suitable therapeutic agents include the Further Chemotherapetic Agents described herein. In the ADC, the antibody or fragment and therapeutic agent are, in an embodiment of the invention, conjugated via a linker, which may be cleavable such as a peptidyl, disulfide, or hydrazone linker, e.g., wherein the linker is a peptidyl linker such as Val-Cit, Ala- Val, Val- Ala- Val, Lys-Lys, Pro-Val-Gly-Val-Val (SEQ ID NO: 160), Ala-Asn-Val, Val-Leu-Lys, Ala-Ala-Asn, Cit-Cit, Val-Lys, Lys, Cit, Ser, or Glu. The ADCs can be prepared as described in U.S. Pat. Nos. 7,087,600; 6,989,452; and 7,129,261; PCT Publications WO 02/096910; WO 07/038658; WO 07/051081; WO 07/059404; WO 08/083312; and WO 08/103693; U.S. Patent Publications 20060024317; 20060004081; and 20060247295; the disclosures of which are incorporated herein by reference.

Any method known in the art for conjugating the antibody molecules and fragments of the invention to the various moieties may be employed, including those methods described by Hunter, et al., (1962) Nature 144:945; David, et al., (1974) Biochemistry 13:1014; Pain, et al., (1981) J. Immunol. Meth. 40:219; and Nygren, J., (1982) Histochem. and Cytochem. 30:407. Methods for conjugating antibodies are conventional and very well known in the art.

The present invention provides a vessel (e.g., a plastic or glass vial, e.g., with a cap) comprising any of the antibodies or antigen-binding fragments, polypeptides or polynucleotides set forth herein or a pharmaceutical composition thereof comprising a pharmaceutically acceptable carrier. The present invention also provides an injection device comprising any of the antibodies or antigen-binding fragments, polypeptides or polynucleotides set forth herein or a pharmaceutical composition thereof. An injection device is a device that introduces a substance into the body of a patient via a parenteral route, e.g., intramuscular, subcutaneous or intravenous. For example, an injection device may be a syringe which, for example, includes a cylinder or barrel for holding fluid to be injected (e.g., antibody or fragment or a pharmaceutical composition thereof), a needle for piecing skin and/or blood vessels for injection of the fluid; and a plunger for pushing the fluid out of the cylinder and through the needle bore. In an embodiment of the invention, an injection device that comprises an antibody or antigen-binding fragment thereof of the present invention or a pharmaceutical composition thereof is a intravenous (IV) injection device. Such a device includes the antibody or fragment or a pharmaceutical composition thereof in a cannula or trocar/needle which may be attached to a tube which may be attached to a bag or reservoir for holding fluid (e.g., saline; or lactated ringer solution comprising NaCl, sodium lactate, KCl, CaCl₂ and optionally including glucose) introduced into the body of the patient through the cannula or trocar/needle. The antibody or fragment or a pharmaceutical composition thereof may, in an embodiment of the invention, be introduced into the device once the trocar and cannula are inserted into the vein of a subject and the trocar is removed from the inserted cannula. The IV device may, for example, be inserted into a peripheral vein (e.g., in the hand or arm); the superior vena cava or inferior vena cava, or within the right atrium of the heart (e.g., a central IV); or into a subclavian, internal jugular, or a femoral vein and, for example, advanced toward the heart until it reaches the superior vena cava or right atrium (e.g., a central venous line). In an embodiment of the invention, an injection device is an autoinjector; a jet injector or an external infusion pump. A jet injector uses a high-pressure narrow jet of liquid which penetrate the epidermis to introduce the antibody or fragment or a pharmaceutical composition thereof to a patient's body. External infusion pumps are medical devices that deliver the antibody or fragment or a pharmaceutical composition thereof into a patient's body in controlled amounts. External infusion pumps may be powered electrically or mechanically. Different pumps operate in different ways, for example, a syringe pump holds fluid in the reservoir of a syringe, and a moveable piston controls fluid delivery, an elastomeric pump holds fluid in a stretchable balloon reservoir, and pressure from the elastic walls of the balloon drives fluid delivery. In a peristaltic pump, a set of rollers pinches down on a length of flexible tubing, pushing fluid forward. In a multi-channel pump, fluids can be delivered from multiple reservoirs at multiple rates.

The present invention provides a kit comprising (a) any of the antibodies or antigen-binding fragments, polypeptides or polynucleotides of the invention the antibody or fragment or a pharmaceutical composition thereof in a vessel or injection device; and (b) a package insert comprising one or more items of information regarding said formulation selected from the group consisting of pharmacokinetics, pharmacodynamics, clinical studies, efficacy parameters, indications and directions for usage, contraindications, warnings, precautions, adverse reactions, overdosage, proper dosage and administration, how supplied, proper storage conditions, references and patent information.

The present invention provides a chromatographic purification resin, e.g., cation-exchange or anion-exchange, bound to any of the antibodies or antigen-binding fragments (e.g., 6C10, 10C11, 6A11, 5B7, 1B7, 5E12, 5G10, 8D12, 2C5, 6F5, 8A12, 1B5, 10B1, 4A2, 14F11, 9F8, 5D10, 1D6 or 1F4), polypeptides or polynucleotides set forth herein. In an embodiment of the invention, the resin is glass, an insoluble polysaccharide, sepharose, agarose or cross-linked polystyrene. The present invention also includes a purification vessel, such as a column, that comprises a purification resin, such as, for example, a cation-exchange resin, an anion-exchange resin, a size exclusion chromatography resin, a high pressure liquid chromatography (HPLC) resin, a fast protein liquid chromatography (FPLC) resin, an affinity purification resin (e.g., protein-A, protein-G, cobalt, nickel, glutathione), that is bound to any of the antibodies or antigen-binding fragments (e.g., 6C10, 10C11, 6A11, 5B7, 1B7, 5E12, 5G10, 8D12, 2C5, 6F5, 8A12, 1B5, 10B1, 4A2, 14F11, 9F8, 5D10, 1 D6 or 1F4), polypeptides or polynucleotides set forth herein.

### Fc Mutants

The present invention includes anti-BLyS antibodies and antigen-binding fragments thereof which comprise one or more sialic acids (including NANA (N-acetylneuraminic acid), NGNA (N-glycolylneuraminic acid), and analogs and derivatives thereof), e.g., enhanced sialylation relative to wild-type antibody sialylation. The invention includes to an anti-BLyS antibodies and antigen-binding fragments thereof of the present invention comprising immunoglobulin constant domains that comprise one or more mutations at amino acid positions 243, 264, 267 and 328, wherein the numbering is according to the EU index as in Kabat. See e.g., Edelman G. M. et al., Proc. Natl. Acad. USA, 63:78-85 (1969); Kabat, E. A. et al., Sequences of proteins of immunological interest. 5^{th} Edition—U.S. Department of Health and Human Services, NIH publication no 91-3242, pp. 662, 680, 689 (1991).

In an embodiment of the invention, the mutations at positions 243 are selected from the group consisting of: F243A, F243G, F243S, F243T, F243V, F243L, F243I, F243D, F243Y, F243E, F243R, F243W and F243K; the mutations at position 264 are selected from the group consisting of: V264A, V264R, V264G, V264S, V264T, V264D, V264E, V264K, V264W, V264H, V264P, V264N, V264Q and V264L; the mutations at position 267 are selected from the group consisting of: S267D, S267Y, S267T; and the mutations at position 328 are selected from the group consisting of L328Y, L328W, L328H.

In an embodiment of the invention, the mutations at positions 243 and 264 are selected from the group consisting of: F243A and V264A; F243Y and V264G; F243T and V264G; F243L and V264A; F243L and V264N; and F243V and V264G. In one embodiment, the mutations are F243A, V264A, S267E, and L328F.

In an embodiment of the invention, the immunoglobulin gamma-1 constant domain comprises the following amino acid sequence (wherein mutated residues are numbered and bolded):

(SEQ ID NO: 161)

ASTKGPSVFP LAPSSKSTSG GTAALGCLVK DYFPEPVTVS
WNSGALTSGV HTFPAVLQSS GLYSLSSVVT VPSSSLGTQT
YICNVNHKPS NTKVDKKVEP KSCDKTHTCP PCPAPELLGG
PSVFLF²⁴³PPKP KDTLMISRTP EVTCVVV²⁶⁴DVS²⁶⁷
HEDPEVKFNW YVDGVEVHNA KTKPREEQYN STYRVVSVLT
VLHQDWLNGK EYKCKVSNKA L³²⁸PAPIEKTIS
KAKGQPREPQ VYTLPPSRDE LTKNQVSLTC LVKGFYPSDI
AVEWESNGQP ENNYKTTPPV LDSDGSFFLY SKLTVDKSRW
QQGNVFSCSV MHEALHNHYT QKSLSLSPGK

In an embodiment of the invention, the immunoglobulin gamma-2 constant domain comprises the following amino acid sequence (wherein mutated residues are numbered and bolded):

(SEQ ID NO: 155)

ASTKGPSVFP LAPCSRSTSE STAALGCLVK DYFPEPVTVS
WNSGALTSGV HTFPAVLQSS GLYSLSSVVT VPSSNFGTQT
YTCNVDHKPS NTKVDKTVER KCCVECPPCP APPVAGPSVF
LF²⁴³PPKPKDTL MISRTPEVTC VVV²⁶⁴DVS²⁶⁷HEDP
EVQFNWYVDG VEVHNAKTKP REEQFNSTFR VVSVLTVVHQ
DWLNGKEYKC KVSNKGL³²⁸PAP IEKTISKTKG QPREPQVYTL
PPSREEMTKN QVSLTCLVKG FYPSDISVEW ESNGQPENNY
KTTPPMLDSD GSFFLYSKLT VDKSRWQQGN VFSCSVMHEA
LHNHYTQKSL SLSPGK

In an embodiment of the invention, the immunoglobulin gamma-4 constant domain comprises the following amino acid sequence (wherein mutated residues are numbered and bolded):

(SEQ ID NO: 156)

ASTKGPSVFP LAPCSRSTSE STAALGCLVK DYFPEPVTVS
WNSGALTSGV HTFPAVLQSS GLYSLSSVVT VPSSSLGTKT
YTCNVDHKPS NTKVDKRVES KYGPPCPSCP APEFLGGPSV
FLF²⁴³PPKPKDT LMISRTPEVT CVVV²⁶⁴DVS²⁶⁷QED
PEVQFNWYVD GVEVHNAKTK PREEQFNSTY RVVSVLTVLH
QDWLNGKEYK CKVSNKGL³²⁸PS SIEKTISKAK GQPREPQVYT
LPPSQEEMTK NQVSLTCLVK GFYPSDIAVE WESNGQPENN
YKTTPPVLDS DGSFFLYSRL TVDKSRWQEG NVFSCSVMHE
ALHNHYTQKS LSLSLGK

In one embodiment of the invention, the N-glycans on anti-BLyS antibodies and antigen-binding fragments thereof of the present invention and sequence variants thereof have a structure selected from SA₍₁₋₄₎Gal₍₁₋₄₎GlcNAc₍₂₋₄₎Man₃GlcNAc₂ and SAGalGlcNAcMan₅GlcNAc₂; e.g.,

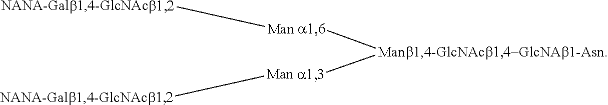

In one embodiment, the immunoglobulin constant domains of the anti-BLyS antibodies and antigen-binding fragments thereof of the invention comprise a mixture of α-2,3 and α-2,6 linked sialic acid. In another embodiment, the anti-BLyS antibodies and antigen-binding fragments thereof of the invention comprise only α-2,6 linked sialic acid. In one embodiment, the anti-BLyS antibodies and antigen-binding fragments thereof of the invention comprise α-2,6 linked sialic acid and comprise no detectable level of α-2,3 linked sialic acid. In one embodiment, the sialic acid is N-acetylneuraminic acid (NANA) or N-glycolylneuraminic acid (NGNA) or a mixture thereof. In another embodiment, the sialic acid is an analog or derivative of NANA or NGNA with acetylation at position 9 on the sialic acid. In one embodiment, the N-glycans on the anti-BLyS antibodies and antigen-binding fragments thereof of the invention comprise NANA and no NGNA. In one embodiment, the N-glycans on the anti-BLyS antibodies and antigen-binding fragments thereof of the invention comprise α-2,6 linked NANA (and no NGNA). In one embodiment, the anti-BLyS antibodies and antigen-binding fragments thereof of the invention comprise sialylated N-glycans comprising a structure selected from SA(₁₋₄)Gal₍₁₋₄₎GlcNAc₍₂₋₄₎Man₃GlcNAc₂ or SAGalGlcNAcMan₅GlcNAc₂, wherein the sialic acid residues are via α-2,6 linkages.

The N-glycans on the anti-BLyS antibodies and antigen-binding fragments thereof of the invention can optionally comprise fucose. In one embodiment, the N-glycans on the anti-BLyS antibodies and antigen-binding fragments thereof will comprise a mixture of fucosylated and non-fucosylated N-glycans. In another embodiment, the N-glycans on the anti-BLyS antibodies and antigen-binding fragments thereof lack fucose. In one embodiment, the immunoglobulin constant domains of the anti-BLyS antibodies and antigen-binding fragments thereof of the invention have one or more of the following properties when compared to an antibody or fragment having a native immunoglobulin constant domain: (i) reduced effector function; (ii) increased anti-inflammatory properties; (iii) increased binding to a lectin (e.g., CD22 (Siglec 2)); (iv) reduced binding to FcγRIIa; (v) increased binding to FcγRIIb; (vi) reduced binding to FcγRIIIa; and (v) reduced binding to FcγRIIIb.

In one embodiment, the anti-BLyS antibodies and antigen-binding fragments thereof of the invention have reduced effector function when compared to an antibody or fragment having a native immunoglobulin constant domain. In one embodiment, the effector function is ADCC (antibody-dependent cellular cytotoxicity). In another embodiment, the effector function is CDC (complement-dependent cytotoxicity). In another embodiment, the effector function is ADCP (antibody-dependent cellular phagocytosis).

In one embodiment, the anti-BLyS antibodies and antigen-binding fragments thereof of the invention has reduced ADCC activity when compared to an antibody or fragment having a native immunoglobulin constant domain. In another embodiment, the anti-BLyS antibodies and antigen-binding fragments thereof has at least a 100 fold reduction in ADCC activity. In another embodiment, the anti-BLyS antibodies and antigen-binding fragments thereof has at least a 500 fold reduction in ADCC activity. In another embodiment, the anti-BLyS antibodies and antigen-binding fragments thereof has at least a 1000 fold reduction in ADCC activity. In one embodiment, the anti-BLyS antibodies and antigen-binding fragments thereof has no detectable ADCC activity.

In one embodiment, the Fc-containing polypeptide of the invention has the following properties when compared to a parent Fc-containing polypeptide: (i) reduced binding to FcγRIIa; (ii) increased binding to FcγRIIb; (iii) reduced binding to FcγRIIIa; and (iv) reduced binding to FcγRIIIb.

In one embodiment, the Fc-containing polypeptide of the invention has the following properties when compared to a parent Fc-containing polypeptide: (i) reduced binding to FcγRIIa; (ii) increased binding to FcγRIIb; and (iii) reduced binding to FcγRIIIa. In one embodiment, an Fc-containing polypeptide of the invention will have no detectable binding to FcγRIIa, FcγRIIIa or FcγRIIIb. In one embodiment, an Fc-containing polypeptide of the invention will have no detectable binding to FcγRIIa, FcγRIIIa FcγRIIIb, when such binding is detected using an ELISA assay.

In one embodiment, the Fc-containing polypeptide of the invention binds FcγRIIb with an increase affinity of at least 2 fold when compared to a parent Fc-containing polypeptide. In one embodiment, the Fc-containing polypeptide of the invention binds FcγRIIb with an increase affinity of at least 4 fold when compared to a parent Fc-containing polypeptide.

In one embodiment, the Fc-containing polypeptide of the invention has increased anti-inflammatory properties compared to a parent Fc-containing polypeptide.

### Therapeutic Methods, Formulations, Dosage and Administration

The present invention provides methods for treating or preventing a medical condition, in a subject, mediated, e.g., directly or indirectly, by BLyS (e.g., BLyS binding to its receptor, e.g., BR3, BCMA or TACI) by administering a therapeutically effective amount of anti-BLyS antibody or antigen-binding fragment thereof to the subject. In an embodiment of the invention, the medical condition is an autoimmune disorder or inflammatory disorder such as appendicitis, peptic ulcer, gastric ulcer and duodenal ulcer, peritonitis, liver steatosis, pancreatitis, inflammatory bowel disease, colitis, ulcerative colitis, pseudomembranous colitis, acute colitis, ischemic colitis, diverticulitis, epiglottitis, achalasia, cholangitis, cholecystitis, coeliac disease, hepatitis, Crohn's disease, enteritis, Whipple's disease, asthma, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, and urethritis, bronchitis, emphysema, rhinitis, fibrosis, cystic fibrosis, pneumonitis, adult respiratory distress syndrome, pneumoultramicroscopicsilicovolcanoconiosis, alvealitis, bronchiolitis, pharyngitis, pleurisy, sinusitis, dermatitis, atopic dermatitis, dermatomyositis, sunburn, urticaria warts, wheals, stenosis, restenosis, vasulitis, angiitis, endocarditis, arteritis, atherosclerosis, thrombophlebitis, pericarditis, myocarditis, myocardial ischemia, periarteritis nodosa, rheumatic fever, meningitis, encephalitis, multiple sclerosis, neuritis, neuralgia, uveitis, arthritides and arthralgias, osteomyelitis, fasciitis, Paget's disease, gout, periodontal disease, rheumatoid arthritis (RA), synovitis, myasthenia gravis, thryoiditis, systemic lupus erythematosus (SLE), goodpasture's syndrome, behcets's syndrome, allograft rejection, graft-versus-host disease, B-cell lymphoma (e.g., non-hodgkins lymphoma), leukemia (e.g., chronic lymphocytic leukemia), granulomatosis with polyangiitis (GPA) (Wegener's granulomatosis), an anti-neutrophil cytoplasmic antibody (ANCA)-associated vasculitide, Churg-Strauss syndrome and/or microscopic polyangiitis.

The term “subject” or “patient” refers to a mammal such as a human (e.g., a human adult or child) or a mouse, rat, rabbit, dog or other canine, horse, goat or primate such as a monkey, chimpanzee or gorilla. In an embodiment of the invention, the subject or patient has anti-neutrophil cytoplasmic antibodies (ANCAs).

The anti-BLyS antibody or antigen-binding fragments thereof discussed herein are, in an embodiment of the invention, administered at a therapeutically effective dosage or amount. The term “therapeutically effective amount” or “therapeutically effective dosage” means that amount or dosage of an agent that will elicit a biological or medical response of a tissue, system, patient or subject that is being sought by the administrator (such as a researcher, doctor or veterinarian) which includes any measurable alleviation of the signs, symptoms and/or clinical indicia of a medical disorder that is mediated by BLyS (e.g., SLE or RA; see above) including the prevention, slowing or halting of progression of the medical disorder to any degree. For example, in one embodiment of the invention, a “therapeutically effective dosage” of any anti-BLyS antibody or antigen-binding fragment thereof discussed herein is between about 0.0001 mg/kg of body weight and about 200 mg/kg of body weight. The therapeutically effective dosage of an anti-BLyS antibody or antigen-binding fragment thereof or any further therapeutic agent is, when possible, as set forth in Physicians Desk Reference 2010; Thomson Reuters; 64 edition (Nov. 15, 2009); and/or in Physicians' Desk Reference 2009; Thomson Reuters; 63rd edition (Nov. 30, 2008) or in the prescribing information of the relevant drug label (if available), such as the U.S. FDA drug label.

The present invention includes methods for using a pharmaceutical composition comprising an anti-BLyS antibody or antigen-binding fragment thereof and a pharmaceutically acceptable carrier. The pharmaceutical compositions may be prepared by any methods well known in the art of pharmacy; see, e.g., Gilman, et al., (eds.) (1990), The Pharmacological Bases of Therapeutics, 8th Ed., Pergamon Press; A. Gennaro (ed.), Remington's Pharmaceutical Sciences, 18th Edition, (1990), Mack Publishing Co., Easton, Pa.; Avis, et al., (eds.) (1993) Pharmaceutical Dosage Forms: Parenteral Medications Dekker, New York; Lieberman, et al., (eds.) (1990) Pharmaceutical Dosage Forms: Tablets Dekker, New York; and Lieberman, et al., (eds.) (1990), Pharmaceutical Dosage Forms: Disperse Systems Dekker, New York.

All routes of administration are contemplated including, but not limited to, parenteral (e.g., subcutaneous, intratumoral, intravenous, intraperitoneal, intramuscular) and non-parenteral (e.g., oral, transdermal, intranasal, intraocular, sublingual, inhalation, rectal and topical). The invention includes a method of administering an antibody or antigen binding fragment thereof that binds specifically to BLyS to a subject comprising introducing the antibody or fragment to the body of the subject by a parenteral (e.g., subcutaneous, intratumoral, intravenous, intraperitoneal, intramuscular) or non-parenteral (e.g., oral, transdermal, intranasal, intraocular, sublingual, inhalation, rectal and topical) route and, optionally, introducing a further chemotherapeutic agent (e.g., as discussed herein) to the body of the subject in association with the antibody or fragment.

Injectables can be prepared in conventional forms, either as liquid solutions or suspensions, solid forms suitable for solution or suspension in liquid prior to injection, or as emulsions. The injectables, solutions and emulsions can also contain one or more excipients. Excipients are, for example, water, sugar, buffer, salt (e.g., NaCl), amino acids (e.g., histidine or glycine), saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

In an embodiment of the invention, pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include sodium chloride injection, ringers injection, isotonic dextrose injection, sterile water injection, dextrose and lactated ringers injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include polysorbate 80 (TWEEN-80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

In an embodiment of the invention, an antibody or antigen-binding fragment thereof (e.g., any one of 6C10, 10C11, 6A11, 5B7, 1B7, 5E12, 5G10, 8D12, 2C5, 6F5, 8A12, 1B5, 10B1, 4A2, 14F11, 9F8, 5D10, 1D6 or 1F4) is in a pharmaceutical composition comprising polysorbate 80.

In an embodiment of the invention, preparations for parenteral administration can include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

The scope of the present invention includes compositions comprising an anti-BLyS antibody or antigen-binding fragment thereof in association with a further chemotherapeutic agent; as well as methods of treating or preventing a medical disorder mediated by BLyS (e.g., an autoimmuno or inflammatory disorder) in a subject comprising administering the antibody or fragment to the subject in association with a further chemotherapeutic agent. In an embodiment of the invention, the further chemotherapeutic agent is: belimumab, tadalumab, denosumab, aspirin, diclofenac, diflunisal, etodolac, fenoprofen, floctafenine, flurbiprofen, ibuprofen, indomethacin, ketoprofen, ketorolac, meclofenamate, mefenamic acid, meloxicam, nabumetone, naproxen, oxaprozin, phenylbutazone, piroxicam, salsalate, sulindac, tenoxicam, tiaprofenic acid, tolmetin, betamethasone benzoate, betamethasone valerate, clobetasol propionate, desoximetasone, fluocinolone acetonide, flurandrenolide, a topical steroid, alclometasone dipropionate, aloe vera, amcinonide, amcinonide, anthralin, betamethasone dipropionate, betamethasone valerate, calcipotriene, clobetasol propionate, coal tar, Dead Sea salts, desonide, desonide; betamethasone valerate, desoximetasone, diflorasone diacetate, epsom salts, fluocinolone acetonide, fluocinonide, flurandrenolide, fluticasone propionate, halcinonide, halobetasol propionate, hydrocortisone valerate, hydrocortisone, mometasone furoate, oilated oatmeal, petroleum jelly, prednicarbate, salicylic acid, tazarotene, triamcinolone acetonide, a mixture of hydrocortisone, dexamethasone, methylprednisolone and prednisolone, alefacept, etanercept, cyclosporine, methotrexate, acitretin, isotretinoin, hydroxyurea, mycophenolate mofetil, sulfasalazine, 6-Thioguanine, anakinra, injectable gold, penicillamine, azathioprine, chloroquine, hydroxychloroquine, sulfasalazine, oral gold, auranofin, gold sodium thiomalate, aurothioglucose, mesalamine, sulfasalazine, budesonide, metronidazole, ciprofloxacin, azathioprine, 6-mercaptopurine or dietary supplementation of calcium, folate, vitamin B12, celecoxib, rofecoxib, valdecoxib, lumiracoxib, etoricoxib, efalizumab, adalimumab, infliximab, rituximab, tocilizumab, or ABX-IL8.

Compositions and methods of the invention include an anti-BLyS antibody or antigen-binding fragment thereof optionally “in association” with one or more further chemotherapeutic agents. The term “in association” indicates that the components of such compositions of the invention can be formulated into a single composition for simultaneous delivery or formulated separately into two or more compositions (e.g., a kit). Furthermore, each component of such a composition of the invention can be administered to a subject at a different time than when the other component is administered; for example, each administration may be given non-simultaneously (e.g., separately or sequentially) at several intervals over a given period of time. Moreover, the separate components may be administered to a subject by the same or by a different route (e.g., orally, intravenously, subcutaneously).

### Host Cell Expression

The present invention includes host cells comprising a polynucleotide encoding a polypeptide of the present invention (e.g,. any of SEQ ID NOs: 1-152), e.g., an immunoglobulin chain of an anti-BLyS antibody or antigen-binding fragment thereof of the present invention, e.g., operably linked to a promoter; as well as methods of use thereof, e.g., methods for expressing the immunoglobulin chains of the antibody or fragment in a host cell. For example, the present invention includes methods for making one or more of the immunoglobulins in a host cell (e.g., Pichia or a Chinese hamster ovary cell) comprising (i) introducing a polynucleotide encoding the polypeptide of the present invention into the host cell and (ii) culturing the host cell in an medium under conditions favorable to expression of the polypeptide in the cell and, optionally, (iii) isolating the immunoglobulin polypeptide from the host cell or culture medium.

In an embodiment of the invention, an isolated fungal host cell of the present invention includes an anti-BLyS immunoglobulin chain (e.g., of 6C10, 10C11, 6A11, 5B7, 1B7, 5E12, 5G10, 8D12, 2C5, 6F5, 8A12, 1B5, 10B1, 4A2, 14F11, 9F8, 5D10, 1D6 or 1F4) and comprises any one or more of the following characteristics:
(i) wherein one or more endogenous beta-mannosyltransferase genes are mutated;
(ii) comprising a polynucleotide encoding an alpha-1,2 mannosidase enzyme;
(iii) wherein one or more endogenous phosphomannosyl transferases are mutated, disrupted, truncated or partially or fully deleted;
(iv) comprising a Leishmania sp. single-subunit oligosaccharyltransferase;
   (v) wherein an endogenous dolichol-P-Man dependent alpha-1,3-mannosyltransferase (e.g., Alg3) is mutated, disrupted, truncated or partially or fully deleted;
(vi) comprising a polynucleotide encoding an endomannosidase;
(vii) comprising one or more polynucleotides encoding a bifunctional UDP-N-acetylglucosamine-2-epimerase/N-acetylmannosamine kinase, an N-acetylneuraminate-9-phosphate synthase, or a CMP-sialic acid synthase;
(viii) wherein endogenous ATT1 gene is mutated, disrupted, truncated or partially or fully deleted;
(ix) wherein an endogenous alpha-1,6-mannosyltransferase (e.g. OCH1) is mutated, disrupted, truncated or partially or fully deleted;
(x) comprising a polynucleotide encoding galactosyltransferase;
(xi) comprising a polynucleotide encoding nucleotide sugar transporter;
(xii) comprising a polynucleotide encoding sialyltransferase;
(xiii) comprising a polynucleotide encoding acetylglucosaminyl transferase;
(xiv) wherein one or more endogenous proteases (e.g., PEP4 and PRB1) are mutated, disrupted, truncated or partially or fully deleted; and/or
(xv) wherein CRZ1 is mutated.

In an embodiment of the invention, an isolated fungal host cell of the present invention includes an anti-BLyS immunoglobulin chain (e.g., of 6C10, 10C11, 6A11, 5B7, 1B7, 5E12, 5G10, 8D12, 2C5, 6F5, 8A12, 1B5, 10B1, 4A2, 14F11, 9F8, 5D10, 1D6 or 1F4) and comprises any one or more of the following alleles:
ura5Δ::ScSUC2;
och1Δ::lacZ;
bmt2Δ::lacZ/KIMNN2-2;
mnn4L1Δ::lacZ/MmSLC35A3;
pno1Δ mnn4A::lacZ;
ADE1::lacZ/NA10/MmSLC35A3/FB8;
his1Δ::lacZ/ScGAL10/XB33/DmUGT;
arg1Δ::HIS1/KD53/TC54;
bmt4Δ::lacZ;
bmt1Δ::lacZ;
bmt3Δ::lacZ;
TRP2::ARG1/MmCST/HsGNE/HsCSS/HsSPS/MmST6-33;
ste13Δ::lacZ-URA5-lacZ/TrMDS1;
dap2Δ::NatR;
TRP5::HygRMmCST/HsGNE/HsCSS/HsSPS/MmST6-33; and/or
att1Δ::ScARR3/LmSTT3D; e.g., all of such alleles.

As used herein, the terms “N-glycan” and “glycoform” are used interchangeably and refer to an N-linked oligosaccharide, e.g., one that is attached by an asparagine-N-acetylglucosamine linkage to an asparagine residue of a polypeptide. N-linked glycoproteins contain an N-acetylglucosamine residue linked to the amide nitrogen of an asparagine residue in the protein. Predominant sugars found on glycoproteins are glucose, galactose, mannose, fucose, N-acetylgalactosamine (GalNAc), N-acetylglucosamine (GlcNAc) and sialic acid (e.g., N-acetyl-neuraminic acid (NANA)).

N-glycans have a common pentasaccharide core of Man₃GlcNAc₂ (“Man” refers to mannose; “Glc” refers to glucose; and “NAc” refers to N-acetyl; GlcNAc refers to N-acetylglucosamine). N-glycans differ with respect to the number of branches (antennae) comprising peripheral sugars (e.g., GlcNAc, galactose, fucose and sialic acid) that are added to the Man₃GlcNAc₂ (“Man₃”) core structure which is also referred to as the “trimannose core”, the “pentasaccharide core” or the “paucimannose core”. N-glycans are classified according to their branched constituents (e.g., high mannose, complex or hybrid). A “high mannose” type N-glycan has five or more mannose residues. A “complex” type N-glycan typically has at least one GlcNAc attached to the 1,3 mannose arm and at least one GlcNAc attached to the 1,6 mannose arm of a “trimannose” core. Complex N-glycans may also have galactose (“Gal”) or N-acetylgalactosamine (“GalNAc”) residues that are optionally modified with sialic acid or derivatives (e.g., “NANA” or “NeuAc”, where “Neu” refers to neuraminic acid and “Ac” refers to acetyl). Complex N-glycans may also have intrachain substitutions comprising “bisecting” GlcNAc and core fucose (“Fuc”). Complex N-glycans may also have multiple antennae on the “trimannose core,” often referred to as “multiple antennary glycans.” A “hybrid” N-glycan has at least one GlcNAc on the terminal of the 1,3 mannose arm of the trimannose core and zero or more mannoses on the 1,6 mannose arm of the trimannose core. The various N-glycans are also referred to as “glycoforms.” “PNGase”, or “glycanase” refer to peptide N-glycosidase F (EC 3.2.2.18).

In an embodiment of the invention, an isolated host cell of the present invention, such as a Pichia cell (e.g., Pichia pastoris), includes one or more polypeptides of the present invention (e.g., SEQ ID NOs: 1-152; e.g., one or more immunoglobulin chains of 6C10, 10C11, 6A11, 5B7, 1B7, 5E12, 5G10, 8D12, 2C5, 6F5, 8A12, 1B5, 10B1, 4A2, 14F11, 9F8, 5D10, 1D6 or 1F4) and is genetically engineered to include a nucleic acid that encodes an α-1,2-mannosidase that has a signal peptide that directs it for secretion. For example, in an embodiment of the invention, the host cell is engineered to express an exogenous α-1,2-mannosidase enzyme having an optimal pH between 5.1 and 8.0, preferably between 5.9 and 7.5. In an embodiment of the invention, the exogenous enzyme is targeted to the endoplasmic reticulum or Golgi apparatus of the host cell, where it trims N-glycans such as Man₈GlcNAc₂ to yield Man₅GlcNAc₂. See U.S. Pat. No. 7,029,872. The present invention includes methods for producing one or more polypeptides of the present invention (e.g., SEQ ID NOs: 1-152; e.g, one or more immunoglobulin chains of 6C10, 10C11, 6A11, 5B7, 1B7, 5E12, 5G10, 8D12, 2C5, 6F5, 8A12, 1B5, 10B1, 4A2, 14F11, 9F8, 5D10, 1D6 or 1F4) comprising (i) introducing a polynucleotide encoding the polypeptide(s) into such a α-1,2-mannosidase⁺ host cell and (ii) culturing the host cell under conditions favorable to expression of the polypeptide(s) in the cell and, optionally, (iii) isolating the polypeptide(s) from the host cell and/or culture medium. The invention also encompasses a method for producing one or more polypeptides of the present invention (e.g., SEQ ID NOs: 1-152; e.g, one or more immunoglobulin chains of 6C10, 10C11, 6A11, 5B7, 1B7, 5E12, 5G10, 8D12, 2C5, 6F5, 8A12, 1B5, 10B1, 4A2, 14F11, 9F8, 5D10, 1D6 or 1F4) comprising an N-glycan structure that comprises a Man₅GlcNAc₂ glycoform in a host cell that does not display alpha-1,6 mannosyltransferase activity with respect to the N-glycan on a glycoprotein, the method comprising the step of introducing into the host cell, a polynucleotide encoding the immunoglobulin glycoprotein, and a polynucleotide encoding an alpha-1,2 mannosidase enzyme selected to have optimal activity in the ER or Golgi of said host cell, the enzyme comprising: (a) an alpha-1,2 mannosidase catalytic domain having optimal activity in said ER or Golgi at a pH between 5.1 and 8.0; fused to (b) a cellular targeting signal peptide not normally associated with the catalytic domain selected to target the mannosidase enzyme to the ER or Golgi apparatus of the host cell; and culturing the fungal host cell under conditions favorable to expression of the polypeptide, whereby, upon expression and passage of the polypeptide through the ER or Golgi apparatus of the host cell, in excess of 30 mole % of the N-glycan structures attached thereto have a Man₅GlcNAc₂ glycoform that can serve as a substrate for GlcNAc transferase I in vivo.

In an embodiment of the invention, an isolated host cell of the present invention comprises one or more polypeptides of the present invention (e.g., SEQ ID NOs: 1-152; e.g., one or more immunoglobulin chains of 6C10, 10C11, 6A11, 5B7, 1B7, 5E12, 5G10, 8D12, 2C5, 6F5, 8A12, 1B5, 10B1, 4A2, 14F11, 9F8, 5D10, 1D6 or 1F4), such as Pichia host cells (e.g., Pichia pastoris) is genetically engineered to eliminate glycoproteins having alpha-mannosidase-resistant N-glycans by deleting or disrupting one or more of the β-mannosyltransferase genes (e.g., BMTI, BMT2, BMT3, and/or BMT4, e.g., BMTI, BMT3, and/or BMT4) (See, U.S. Pat. No. 7,465,577) or abrogating translation of RNAs encoding one or more of the beta-mannosyltransferases using interfering RNA, antisense RNA, or the like. The scope of the present invention includes methods for producing one or more polypeptides of the present invention (e.g., SEQ ID NOs: 1-152; e.g., one or more immunoglobulin chains of 6C10, 10C11, 6A11, 5B7, 1B7, 5E12, 5G10, 8D12, 2C5, 6F5, 8A12, 1B5, 10B1, 4A2, 14F11, 9F8, 5D10, 1D6 or 1F4) comprising (i) introducing a polynucleotide encoding the polypeptide(s) into such a β-mannosyltransferase⁻ (bmt1⁻, bmt2⁻, bmt3, and/or bmt4⁻, e.g., bmt1⁻, bmt3, and/or bmt4) host cell and (ii) culturing the host cell under conditions favorable to expression of the polypeptide(s) in the cell and, optionally, (iii) isolating the polypeptide(s) from the host cell and/or culture medium.

In an embodiment of the invention, an isolated fungal host cell (e.g., Pichia, e.g., Pichia pastoris) of the present invention comprises an one or more polypeptides of the present invention (e.g., SEQ ID NOs: 1-152; e.g., one or more immunoglobulin chains of 6C10, 10C11, 6A11, 5B7, 1B7, 5E12, 5G10, 8D12, 2C5, 6F5, 8A12, 1B5, 10B1, 4A2, 14F11, 9F8, 5D10, 1 D6 or 1F4) and also is genetically engineered to eliminate glycoproteins having phosphomannose residues, e.g., by deleting or disrupting one or both of the phosphomannosyl transferase genes PNO1 and MNN4B (See for example, U.S. Pat. Nos. 7,198,921 and 7,259,007), which can include deleting or disrupting one or more of the phosphomannosyltransferases or abrogating translation of RNAs encoding one or more of the phosphomannosyltransferases using interfering RNA, antisense RNA, or the like. In an embodiment of the invention, such fungal host cells produce such immunoglobulnis that have predominantly an N-glycan selected from the group consisting of complex N-glycans, hybrid N-glycans, and high mannose N-glycans wherein complex N-glycans are, in an embodiment of the invention, selected from the group consisting of Man₃GlcNAc₂, GlcNAC_{(I-4)}Man₃GlcNAc₂, NANA₍₁₋₄₎GlcNAc_{(I-4)}Man₃GlcNAc₂, and NANA_{(I-4)}Gal₍₁₋₄₎Man₃GlcNAc₂; hybrid N-glycans are, in an embodiment of the invention, selected from the group consisting of Man₅GlcNAc₂, GlcNAcMan₅GlcNAc₂, GalGlcNAcMan₅GlcNAc₂, and NANAGalGlcNAcMan₅GlcNAc₂; and high mannose N-glycans are, in an embodiment of the invention, selected from the group consisting of Man₆GlcNAc₂, Man₇GlcNAc₂, Mang₈1cNAc₂, and Man₉GlcNAc₂. The scope of the present invention includes methods for producing one or more polypeptides of the present invention (e.g., SEQ ID NOs: 1-152; e.g., one or more immunoglobulin chains of 6C10, 10C11, 6A11, 5B7, 1B7, 5E12, 5G10, 8D12, 2C5, 6F5, 8A12, 1B5, 10B1, 4A2, 14F11, 9F8, 5D10, 1D6 or 1F4) comprising (i) introducing a polynucleotide encoding the polypeptide(s) into such a phosphomannosyl transferase⁻ (e.g., pno1⁻ and/or mnn4b⁻) host cell and (ii) culturing the host cell under conditions favorable to expression of the polypeptide(s) in the cell and, optionally, (iii) isolating the polypeptide(s) from the host cell and/or culture medium.

In an embodiment of the invention, an isolated host cell, such as Pichia host cells (e.g., Pichia pastoris) of the present invention includes one or more polypeptides of the present invention (e.g., SEQ ID NOs: 1-152; e.g., one or more immunoglobulin chains of 6C10, 10C11, 6A11, 5B7, 1B7, 5E12, 5G10, 8D12, 2C5, 6F5, 8A12, 1B5, 10B1, 4A2, 14F11, 9F8, 5D10, 1D6 or 1F4) and is genetically engineered to include a nucleic acid that encodes the Leishmania sp. single-subunit oligosaccharyltransferase STT3A protein, STT3B protein, STT3C protein, STT3D protein, or combinations thereof such as those described in international application publication no. WO2011/06389. The scope of the present invention includes methods for producing one or more polypeptides of the present invention (e.g., SEQ ID NOs: 1-152; e.g., one or more immunoglobulin chains of 6C10, 10C11, 6A11, 5B7, 1B7, 5E12, 5G10, 8D12, 2C5, 6F5, 8A12, 1B5, 10B1, 4A2, 14F11, 9F8, 5D10, 1D6 or 1F4) comprising (i) introducing a polynucleotide encoding the polypeptide(s) into such a (Leishmania STT3A⁺, Leishmania STT3B⁺, Leishmania STT3C⁺, and/or Leishmania STT3D⁺) host cell and (ii) culturing the host cell under conditions favorable to expression of the polypeptide(s) in the cell and, optionally, (iii) isolating the polypeptide(s) from the host cell and/or culture medium.

In an embodiment of the invention, an isolated host cell (e.g., Pichia pastoris) of the present invention includes one or more polypeptides of the present invention (e.g., SEQ ID NOs: 1-152; e.g., one or more immunoglobulin chains of 6C10, 10C11, 6A11, 5B7, 1B7, 5E12, 5G10, 8D12, 2C5, 6F5, 8A12, 1B5, 10B1, 4A2, 14F11, 9F8, 5D10, 1D6 or 1F4) and also is genetically engineered to eliminate nucleic acids encoding dolichol-P-Man dependent alpha(1-3) mannosyltransferase, e.g., Alg3, such as described in U.S. Patent Publication No. US2005/0170452. The scope of the present invention includes methods for producing one or more polypeptides of the present invention (e.g., SEQ ID NOs: 1-152; e.g., one or more immunoglobulin chains of 6C10, 10C11, 6A11, 5B7, 1B7, 5E12, 5G10, 8D12, 2C5, 6F5, 8A12, 1B5, 10B1, 4A2, 14F11, 9F8, 5D10, 1D6 or 1F4) comprising (i) introducing a polynucleotide encoding the polypeptide(s) into such a Alg3⁻ host cell and (ii) culturing the host cell under conditions favorable to expression of the polypeptide(s) in the cell and, optionally, (iii) isolating the polypeptide(s) from the host cell and/or culture medium.

In an embodiment of the invention, an isolated fungal host cell of the present invention, such as Pichia cells (e.g., Pichia pastoris) includes one or more polypeptides of the present invention (e.g., SEQ ID NOs: 1-152; e.g., one or more immunoglobulin chains of 6C10, 10C11, 6A11, 5B7, 1B7, 5E12, 5G10, 8D12, 2C5, 6F5, 8A12, 1B5, 10B1, 4A2, 14F11, 9F8, 5D10, 1D6 or 1F4) and expresses a polypeptide having an endomannosidase activity (e.g., human (e.g., human liver), rat or mouse endomannosidase) that is targeted to a vesicular compartment within the host cell are part of the present invention. The scope of the present invention includes methods for producing one or more polypeptides of the present invention (e.g., SEQ ID NOs: 1-152; e.g., one or more immunoglobulin chains of 6C10, 10C11, 6A11, 5B7, 1B7, 5E12, 5G10, 8D12, 2C5, 6F5, 8A12, 1B5, 10B1, 4A2, 14F11, 9F8, 5D10, 1D6 or 1F4) comprising (i) introducing a polynucleotide encoding the polypeptide(s) into such a endomannosidase⁺ host cell and (ii) culturing the host cell under conditions favorable to expression of the polypeptide(s) in the cell and, optionally, (iii) isolating the polypeptide(s) from the host cell and/or culture medium.

In an embodiment of the invention, an isolated host cell, such as Pichia cells (e.g., Pichia pastoris) of the present invention includes one or more polypeptides of the present invention (e.g., SEQ ID NOs: 1-152; e.g., one or more immunoglobulin chains of 6C10, 10C11, 6A11, 5B7, 1B7, 5E12, 5G10, 8D12, 2C5, 6F5, 8A12, 1B5, 10B1, 4A2, 14F11, 9F8, 5D10, 1 D6 or 1F4) and is engineered for producing a recombinant sialylated glycoprotein in the host cell, e.g., wherein the host cell is selected or engineered to produce recombinant glycoproteins comprising a glycoform selected from the group consisting of Gal(₁₋₄)GlcNAc₍₁₋₄₎Man₃GlcNAc₂, e.g., by a method comprising: (a) transforming, into the host cell, one or more polynucleotides encoding a bifunctional UDP-N-acetylglucosamine-2-epimerase/N-acetylmannosamine kinase, an N-acetylneuraminate-9-phosphate synthase, and a CMP-sialic acid synthase; (b) transforming into the host cell a polynucleotide encoding a CMP-sialic acid transporter; and (c) transforming into the host cell a polynucleotide molecule encoding a 2,6-sialyltransferase catalytic domain fused to a cellular targeting signal peptide, e.g., encoded by nucleotides 1-108 of the S. cerevisiae Mnn2; wherein, upon passage of a recombinant glycoprotein through the secretory pathway of the host cell, a recombinant sialylated glycoprotein comprising a glycoform selected from the group consisting of NANA₍₁₋₄₎Gal₍₁₋₄₎Gal₍₁₋₄₎GlcNAc₍₁₋₄₎Man₃GlcNAc₂ glycoform is produced. The scope of the present invention includes methods for producing one or more polypeptides of the present invention (e.g., SEQ ID NOs: 1-152; e.g., one or more immunoglobulin chains of 6C10, 10C11, 6A11, 5B7, 1B7, 5E12, 5G10, 8D12, 2C5, 6F5, 8A12, 1B5, 10B1, 4A2, 14F11, 9F8, 5D10, 1D6 or 1F4) comprising (i) introducing a polynucleotide encoding the polypeptide(s) into such a bifunctional UDP-N-acetylglucosamine-2-epimerase/N-acetylmannosamine kinase⁺, N-acetylneuraminate-9-phosphate synthase⁺, CMP-Sialic acid synthase⁺, CMP-sialic acid transporter⁺, 2,6-sialyltransferase⁺ fungal host cell and (ii) culturing the host cell under conditions favorable to expression of the polypeptide(s) in the cell and, optionally, (iii) isolating the polypeptide(s) from the host cell and/or culture medium.

In an embodiment of the invention, an isolated host cell of the present invention, such as Pichia cells (e.g., Pichia pastoris), includes one or more polypeptides of the present invention (e.g., SEQ ID NOs: 1-152; e.g., one or more immunoglobulin chains of 6C10, 10C11, 6A11, 5B7, 1B7, 5E12, 5G10, 8D12, 2C5, 6F5, 8A12, 1B5, 10B1, 4A2, 14F11, 9F8, 5D10, 1D6 or 1F4) and is engineered for generating galactosylated proteins, e.g., having a terminal galactose residue and essentially lacking fucose and sialic acid residues on the glycoprotein. In one embodiment of the present invention, the isolated host cell comprises an isolated nucleic acid molecule encoding β-galactosyltransferase activity and at least a polynucleotide encoding UDP-galactose transport activity, UDP-galactose C4 epimerase activity, galactokinase activity or galactose-1-phosphate uridyl transferase, e.g., wherein the host cell is genetically engineered to produce N-linked oligosaccharides having terminal GlcNAc residues and comprising a polynucleotide encoding a fusion protein that in the host cell transfers a galactose residue from UDP-galactose onto a terminal GlcNAc residue of an N-linked oligosaccharide branch of an N-glycan of a glycoprotein, wherein the N-linked oligosaccharide branch is selected from the group consisting of GlcNAcβ1,2-Manα1; GlcNAcβ1,4-Manα1,3, GlcNAcβ1,2-Manα1,2, GlcNAcβ1,4-Manα1,6 and GlcNAcβ1,6-Manα1,6; wherein the host cell is diminished or depleted in dolichyl-P-Man:Man₅GlcNAc₂-PP-dolichyl α-1,3 mannosyltransferase activity, and wherein the host cell produces a glycoprotein having one or more galactose residues. The scope of the present invention includes methods for producing one or more polypeptides of the present invention (e.g., SEQ ID NOs: 1-152; e.g., one or more immunoglobulin chains of 6C10, 10C11, 6A11, 5B7, 1B7, 5E12, 5G10, 8D12, 2C5, 6F5, 8A12, 1B5, 10B1, 4A2, 14F11, 9F8, 5D10, 1D6 or 1F4) comprising (i) introducing a polynucleotide encoding the polypeptide(s) into such a host cell and (ii) culturing the host cell under conditions favorable to expression of the polypeptide(s) in the cell and, optionally, (iii) isolating the polypeptide(s) from the host cell and/or culture medium.

In an embodiment of the invention, an isolated host cell of the present invention, such as Pichia cells (e.g., Pichia pastoris) includes one or more polypeptides of the present invention (e.g., SEQ ID NOs: 1-152; e.g., one or more immunoglobulin chains of 6C10, 10C11, 6A11, 5B7, 1B7, 5E12, 5G10, 8D12, 2C5, 6F5, 8A12, 1B5, 10B1, 4A2, 14F11, 9F8, 5D10, 1D6 or 1F4) and lacks functional ATT1, OCH1 and/or CRZ1 protein, e.g., wherein endogenous ATT1, OCH1 and/or CRZ1 is mutated (e.g., wherein the CRZ1 mutation selected from L33→STOP; Q214→STOP; L294→STOP; S298→STOP; E403→G; F406→S; F406→L; C411→F; and K469→N). The scope of the present invention includes methods for producing one or more polypeptides of the present invention (e.g., SEQ ID NOs: 1-152; e.g., one or more immunoglobulin chains of 6C10, 10C11, 6A11, 5B7, 1B7, 5E12, 5G10, 8D12, 2C5, 6F5, 8A12, 1B5, 10B1, 4A2, 14F11, 9F8, 5D10, 1D6 or 1F4) comprising (i) introducing a polynucleotide encoding the polypeptide(s) into such a och1⁻, and/or crz1⁻ and/or att1⁻ host cell and (ii) culturing the host cell under conditions favorable to expression of the polypeptide(s) in the cell and, optionally, (iii) isolating the polypeptide(s) from the host cell and/or culture medium.

In an embodiment of the invention, an isolated host cell of the present invention, such as Pichia cells (e.g., Pichia pastoris) includes one or more polypeptides of the present invention (e.g., SEQ ID NOs: 1-152; e.g., one or more immunoglobulin chains of 6C10, 10C11, 6A11, 5B7, 1B7, 5E12, 5G10, 8D12, 2C5, 6F5, 8A12, 1B5, 10B1, 4A2, 14F11, 9F8, 5D10, 1D6 or 1F4) and expresses a galactosyltransferase e.g., an alpha 1,3-galactosyltransferase or a beta 1,4-galactosyltransferase. The scope of the present invention includes methods for producing one or more polypeptides of the present invention (e.g., SEQ ID NOs: 1-152; e.g., one or more immunoglobulin chains of 6C10, 10C11, 6A11, 5B7, 1B7, 5E12, 5G10, 8D12, 2C5, 6F5, 8A12, 1B5, 10B1, 4A2, 14F11, 9F8, 5D10, 1D6 or 1F4) comprising (i) introducing a polynucleotide encoding the polypeptide(s) into such a galactosyltransferase⁺ host cell and (ii) culturing the host cell under conditions favorable to expression of the polypeptide(s) in the cell and, optionally, (iii) isolating the polypeptide(s) from the host cell and/or culture medium.

In an embodiment of the invention, an isolated host cell of the present invention, such as Pichia cells (e.g., Pichia pastoris) includes one or more polypeptides of the present invention (e.g., SEQ ID NOs: 1-152; e.g., one or more immunoglobulin chains of 6C10, 10C11, 6A11, 5B7, 1B7, 5E12, 5G10, 8D12, 2C5, 6F5, 8A12, 1B5, 10B1, 4A2, 14F11, 9F8, 5D10, 1D6 or 1F4) and expresses a nucleotide sugar transporter. The scope of the present invention includes methods for producing one or more polypeptides of the present invention (e.g., SEQ ID NOs: 1-152; e.g., one or more immunoglobulin chains of 6C10, 10C11, 6A11, 5B7, 1B7, 5E12, 5G10, 8D12, 2C5, 6F5, 8A12, 1B5, 10B1, 4A2, 14F11, 9F8, 5D10, 1D6 or 1F4) comprising (i) introducing a polynucleotide encoding the polypeptide(s) into such a nucleotide sugar transporter⁺ host cell and (ii) culturing the host cell under conditions favorable to expression of the polypeptide(s) in the cell and, optionally, (iii) isolating the polypeptide(s) from the host cell and/or culture medium.

In an embodiment of the invention, an isolated host cell of the present invention, such as Pichia cells (e.g., Pichia pastoris) includes one or more polypeptides of the present invention (e.g., SEQ ID NOs: 1-152; e.g., one or more immunoglobulin chains of 6C10, 10C11, 6A11, 5B7, 1B7, 5E12, 5G10, 8D12, 2C5, 6F5, 8A12, 1B5, 10B1, 4A2, 14F11, 9F8, 5D10, 1D6 or 1F4) and expresses a sialyltransferase. The scope of the present invention includes methods for producing one or more polypeptides of the present invention (e.g., SEQ ID NOs: 1-152; e.g., one or more immunoglobulin chains of 6C10, 10C11, 6A11, 5B7, 1B7, 5E12, 5G10, 8D12, 2C5, 6F5, 8A12, 1B5, 10B1, 4A2, 14F11, 9F8, 5D10, 1D6 or 1F4) comprising (i) introducing a polynucleotide encoding the polypeptide(s) into such a sialyltransferase⁺ host cell and (ii) culturing the host cell under conditions favorable to expression of the polypeptide(s) in the cell and, optionally, (iii) isolating the polypeptide(s) from the host cell and/or culture medium.

In an embodiment of the invention, an isolated host cells of the present invention, such as Pichia cells (e.g., Pichia pastoris) includes one or more polypeptides of the present invention (e.g., SEQ ID NOs: 1-152; e.g., one or more immunoglobulin chains of 6C10, 10C11, 6A11, 5B7, 1B7, 5E12, 5G10, 8D12, 2C5, 6F5, 8A12, 1B5, 10B1, 4A2, 14F11, 9F8, 5D10, 1D6 or 1F4) and expresses an acetylglucosaminyl transferase, e.g., GNT1 or GNT2 or GNT4. The scope of the present invention includes methods for producing one or more polypeptides of the present invention (e.g., SEQ ID NOs: 1-152; e.g., one or more immunoglobulin chains of 6C10, 10C11, 6A11, 5B7, 1B7, 5E12, 5G10, 8D12, 2C5, 6F5, 8A12, 1B5, 10B1, 4A2, 14F11, 9F8, 5D10, 1D6 or 1F4) comprising (i) introducing a polynucleotide encoding the polypeptide(s) into such an acetylglucosaminyl transferase⁺ host cell and (ii) culturing the host cell under conditions favorable to expression of the polypeptide(s) in the cell and, optionally, (iii) isolating the polypeptide(s) from the host cell and/or culture medium.

As used herein, the term “essentially free of” as it relates to lack of a particular sugar residue, such as fucose, or galactose or the like, on a glycoprotein, is used to indicate that the glycoprotein composition is substantially devoid of N-glycans which contain such residues. Expressed in terms of purity, essentially free means that the amount of N-glycan structures containing such sugar residues does not exceed 10%, and preferably is below 5%, more preferably below 1%, most preferably below 0.5%, wherein the percentages are by weight or by mole percent.

As used herein, a glycoprotein composition “lacks” or “is lacking” a particular sugar residue, such as fucose or galactose, when no detectable amount of such sugar residue is present on the N-glycan structures. For example, in an embodiment of the present invention, glycoprotein compositions produced by host cells of the invention will “lack fucose,” because the cells do not have the enzymes needed to produce fucosylated N-glycan structures. Thus, the term “essentially free of fucose” encompasses the term “lacking fucose.” However, a composition may be “essentially free of fucose” even if the composition at one time contained fucosylated N-glycan structures or contains limited, but detectable amounts of fucosylated N-glycan structures as described above.

### EXAMPLES

The following information is provided for more clearly describing the present invention and should not be construed to limit the present invention. All compositions and methods described below fall within the scope of the present invention.

### Example 1

### Identification of Neutralizing Human Anti-BLyS Antibodies Using Phage Display

Antibodies that bound to BLyS were identified and characterized as set forth herein.

Materials and Methods.

A naïve phage display library from a diverse collection of healthy individuals was constructed and used to isolate fully human antibodies targeting human BLyS. During library construction, all the V-genes were amplified with high fidelity and each family member individually cloned and assembled to match the natural human immune repertoire to maximize the diversity of the library. Multiple strategies of enriching for human BLyS binding antibodies were employed, including solid-phase panning using immobilized antigen and solution-phase panning using biotinylated antigen. For each strategy, multiple rounds of panning were used to enrich for BLyS binding clones and the scFv fragments from the eluted phage were expressed in a standard E. coli bacterial strain and periplasmic extracts (PPE) were generated to assay for binding. Each scFv clone was screened for binding to soluble human, mouse and cynomolgus BLyS by standard ELISA detection methods and membrane bound BLyS on either CHO-K1 or HEK293 cells recombinantly expressing human, mouse and cynogmolus BLyS by standard FACS analysis. BLyS expression naturally occurs as a membrane bound form and requires enzymatic cleavage by furin to get released into circulation so binding to both forms was sought.

Antibody clones showing positive binding to both soluble and membrane bound human BLyS were further tested for the ability to block binding of soluble BLyS to a soluble form of the BLyS receptor 3, BR3. BR3 was immobilized in wells of a microtiter plate and the binding of biotinylated soluble BLyS in the presence of the scFv clones was measured to determine whether the antibodies could block that interaction. In parallel, clones were also assayed for off-rates (k_{off}) to soluble human BLyS using the Fortebio Octet RED system to help identify the potentially highest affinity antibodies. scFv clones were captured onto an NTA biosensor and soluble BLyS was allowed to associate for 2-5 minutes followed by a 5 minute dissociation phase to estimate the dissociation rate.

Antibodies that bound to soluble and membrane bound human BLyS and were able to block binding of soluble human BLyS to soluble BR3 were reformatted to IgG for further characterization. Clones that also showed cross reactivity to soluble and membrane bound mouse BLyS and/or soluble and membrane bound cynomolgus BLyS were also reformatted to IgG for extended characterization. The IgG reformatted clones were transiently expressed in HEK293-FreeStyle cells using the manufacturers recommended protocol. After 7-10 days of culturing post-transfection, supernatants were harvested and the recombinantly expressed IgGs were purified using Protein A affinity chromatography. The IgGs were re-screened for binding to soluble human, mouse and cynomolgus BLyS by ELISA, membrane bound human, mouse and cynomolgus BLyS by FACS, receptor blocking activity, affinity determination by Biacore and neutralization of BLyS-mediated B-cell proliferation. The data generated by this characterization are set forth in Table 1 below.

TABLE 1
Initial characterization of anti-BLyS clones

| | Soluble | Soluble | Soluble | hBLyS mBR3 | hBLyS | mBLyS |
|---|---|---|---|---|---|---|
| Clone | hBLyS ELISA | mBLyS ELISA | cyBLyS ELISA | Receptor | Biacore K_{D} | Biacore K_{D} |
| ID | (IC₅₀ nM) | (IC₅₀ nM) | (IC₅₀ nM) | Blocking (nM) | (M) | (M) |
| 5B7 | 0.10 | 0.16 | 0.08 | 0.43 | 4.00E−10 | 1.45E−11 |
| 10C11 | 0.10 | 0.04 | 0.07 | 0.07 | 4.30E−10 | 3.13E−11 |
| 6C10 | 0.12 | 0.05 | 0.10 | 0.33 | 5.10E−10 | 2.16E−11 |
| 1B7 | 0.10 | 0.13 | 0.10 | 0.91 | 1.73E−09 | 1.27E−11 |
| 8A12 | 16 | 0.05* | 0.32 | 2.97* | 4.26E−09 | 2.24E−11 |
| 9F8 | 0.15 | 0.07 | 0.07 | 5.31 | 1.10E−09 | 4.56E−11 |
| 8D12 | 0.13 | 0.16 | 0.08 | 10 | 6.60E−10 | 2.80E−11 |
| 5E12 | 33 | 9.36 | | >100 | | |
| 1B5 | 15 | 17 | | 21 | | |
| 5G10 | 62 | 51 | 14.27 | | NB | |
| 10B1 | >100 | NB | | | | |
| IF4 | 2.87 | >100 | 0.11 | | NB | |
| 1D6 | >100 | NB | | >100 | | |
| 6F5 | >100 | | | >100 | | |
| 4A2 | 28 | 0.47 | 1.46 | >100 | NB | 4.86E−11 |
| 2C5 | >100 | NB | | >100 | | |
| 2E10 | >100 | 4.15 | | >100 | | |
| 5D10 | >100 | | | NA | | |
| 6A11 | >100 | | | NA | | |
| 14F11 | NB | >100 | | NA | | |

| | cyBLyS | hBLyS B-cell | mBLyS B-cell | cyBLyS B-cell | CHO- | HEK293 | CHO- |
|---|---|---|---|---|---|---|---|
| Clone | Biacore K_{D} | proliferation | proliferation | proliferation | hBLyS | mBLyS | cyBLyS |
| ID | (nM) | (IC₅₀) | (IC₅₀) | (IC₅₀) | FACS | FACS | FACS |
| 5B7 | | 0.04 | 0.04 | 0.07 | (+) | (+) | (+) |
| 10C11 | | 0.08 | 6.04 | 0.06 | (+) | (+) | (+) |
| 6C10 | | 0.14 | 41 | 0.05 | (+) | (+) | (+) |
| 1B7 | | 0.05 | 0.32 | 0.09 | (+) | (+) | (+) |
| 8A12 | | 0.25 | 3.13 | | (+) | (+) | (+/−) |
| 9F8 | | 3.51 | 0.98 | 9.10 | (+) | (+) | (+) |
| 8D12 | | 1.15 | 25 | 3.20 | (+) | (+) | (+) |
| 5E12 | | 3.29 | 96 | | | | |
| 1B5 | | 0.24 | NA | | | | |
| 5G10 | | 75 | 43 | | (+/−) | (−) | (−) |
| 10B1 | | 5.07 | NA | | | | |
| IF4 | | 2.23 | 53 | | (+/−) | (−) | (−) |
| 1D6 | | 7.44 | NA | | | | |
| 6F5 | | 0.43 | NA | | | | |
| 4A2 | | 6.74 | 3.54 | | (−) | (+) | (−) |
| 2C5 | | 3.41 | NA | | | | |
| 2E10 | | 10 | NA | | | | |
| 5D10 | | 22 | NA | | | | |
| 6A11 | | 2.70 | NA | | | | |
| 14F11 | | 12 | NA | | | | |

### Example 2

### Construction of 243/264 IgG1 Fc Mutants of Anti-BLyS Antibodies

Construction of Anti-BLyS mAbs F243A/V264A Double Mutein (DM) Pichia Pastoris Recombinant Expression Vector.

The preparation of double Fc muteins (DM) of anti-BLyS IgG1 DM monoclonal antibodies in Pichia pastoris was carried out using the sequences listed herein and protocols listed below:

A. Assembled Variable Regions Heavy and Light Chains with Constant Regions—

The variable regions of heavy and light chain sequences identified by phage display from fragment of antigen binding (Fab) of anti-BLyS monoclonal IgG1 antibodies were assembled with constant regions of heavy chain and light chain sequences. Each variable region of anti-BLyS mAbs were assembled with IgG1 heavy chain constant region including CH1, CH2 and CH3 domains. In addition, the position of F243 at Fc was changed into alanine and V264 was modified into alanine. The variable region of light chain sequences were assemble with light constant region (CL). The type of light chain constant regions, either kappa or lamda, were chosen based on BLAST of the variable region of anti-BLyS against the IMGT database.

B. Signal Sequence—

The signal sequence of an α-Mating Factor pre-domain was fused in-frame to the 5′ end of the light or heavy chain. A Kozak sequence AAACG (SEQ ID NO: 157) was added to the 5′ end of the methionine and an EcoR1 site was added before the Kozak sequence for cloning purposes. The amino acid as shown below. Amino acid sequence of alpha pre-domain is M R F P S I F T A V L F A A S S A L A (SEQ ID NO: 158) and DNA sequence including Kozak sequence is ggaaacgatgcgtttcccatccattttcaccgccgtcttgttcgctgcctcttctgccttggcc (SEQ ID NO: 159).

C. Construction of Expression Plasmids for Anti-BLyS IgG1DM—

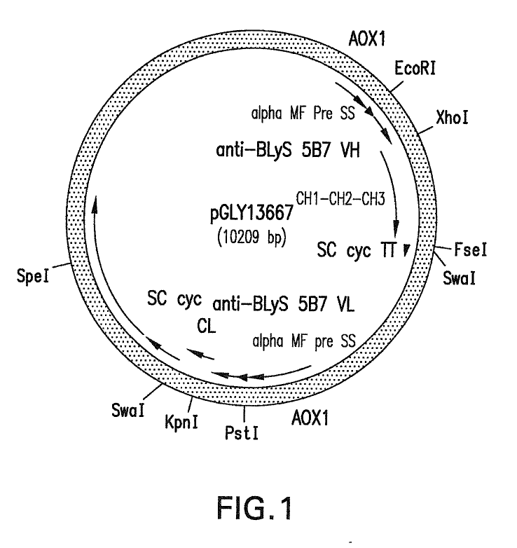

The heavy and light chains with the fused signal sequence of IgG1DM were codon optimized using Pichia pastoris preferred codon usage and reverse translated into DNA and cloned into Pichia monoclonal antibody expression vector. DNA sequences encoding the heavy chain and light chain were cloned into a single vector by a standard cloning method under inducible promoter AOX1 and in front of S. cerevisiae Cyc terminator, respectively. Heavy and light chains DNA sequences were synthesized and cloned through CRO Genewiz Inc. (GENEWIZ Global Headquarters 115 Corporate Boulevard South Plainfield, N.J. 07080). Anti-BLyS mAbs expression plasmids were linearized by restriction enzyme Spe1 digestion and electroporated into Pichia pastoris and integrated into the Trp2 locus. An example of an expression plasmid used to express antibody 5B7 was pGLY13667; see plasmid map in FIG. 1.

### Example 3

### Glycoengineered Pichia GFI6.0 Hosts for Producing Anti-BLyS Monoclonal Antibodies

Following the procedures disclosed in Gerngross, U.S. Pat. No. 7,029,872 and Gerngross, U.S. Pat. No. 7,449,308, vectors were constructed that are useful for genetically engineering lower eukaryotic host cells such that they were capable of expressing a desired polypeptide having a desired N-glycoform as the predominant species. GFI6.0 strains were engineered from NRRL11430 (American Type Culture Collection (ATCC), P.O. Box 1549, Manassas, Va. 20108, USA) according to the methods described in Hamilton et al., Science, 313: 1441-1443 (2006) and Hamilton US200610286637. The engineered Pichia pastoris strain, GFI6.0, is capable of producing proteins with a biantennary N-glycan structure with terminal sialic acid. The GFI6.0 genotype was as follows:
ura5Δ::ScSUC2 och1Δ::lacZ bmt2Δ::lacZIKIMNN2-2
mnn4L1Δ::lacZIMmSLC35A3 pno1Δ mnn4Δ::lacZ
ADE1::lacZ/NA10/MmSLC35A3/FB8
his1Δ::lacZ/ScGAL10/XB33/DmUGT
arg1Δ::HIS1/KD53/TC54
bmt4Δ::lacZ bmt1Δ::lacZ bmt3Δ::lacZ
TRP2::ARG 1/MmCST/HsGNE/HsCSS/HsSPS/MmST6-33
ste13A::lacZ-URA5-lacZITrMDS1 dap2Δ::NatR
TRP5::HygRMmCST/HsGNE/HsCSS/HsSPS/MmST6-33
att1Δ::ScARR3/LmSTT3D
   YGLY28423 is the GFI 6.0 empty host that was used for anti-BLyS mAbs expression.

The abbreviations used to describe the genotypes are commonly known and understood by those skilled in the art, and include the following abbreviations:
ScSUC2 S. cerevisiae Invertase
OCH1 Alpha-1,6-mannosyltransferase
KIMNN2-2 K. lactis UDP-GlcNAc transporter
BMT1 Beta-mannose-transfer (beta-mannose elimination)
BMT2 Beta-mannose-transfer (beta-mannose elimination)
BMT3 Beta-mannose-transfer (beta-mannose elimination)
BMT4 Beta-mannose-transfer (beta-mannose elimination)
MNN4L1 MNN4-like 1 (charge elimination)
MmSLC35A3 Mouse homologue of UDP-GlcNAc transporter
PNO1 Phosphomannosylation of N-glycans (charge elimination)
MNN4 Mannosyltransferase (charge elimination)
ScGAL10 UDP-glucose 4-epimerase
XB33 Truncated HsGalT1 fused to ScKRE2 leader
DmUGT UDP-Galactose transporter
KD53 Truncated DmMNSII fused to ScMNN2 leader
TC54 Truncated RnGNTII fused to ScMNN2 leader
NA10 Truncated HsGNTI fused to PpSEC12 leader
FB8 Truncated MmMNS1A fused to ScSEC12 leader
TrMDS1 Secreted T. reseei MNS1
ADE1 N-succinyl-5-aminoimidazole-4-carboxamide ribotide (SAICAR) synthetase
MmCST Mouse CMP-sialic acid transporter
HsGNE Human UDP-GlcNAc 2-epimerase/N-acetylmannosamine kinase
HsCSS Human CMP-sialic acid synthase
HsSPS Human N-acetylneuraminate-9-phosphate synthase
MmST6-33 Truncated Mouse alpha-2,6-sailyl transferase fused to ScKRE2 leader
LmSTT3d Catalytic subunit of oligosaccharyltransferase from Leishmania major

### Example 4

### Yeast Transformation and Screening

The glycoengineered GFI 6.0 strain, YGLY28423, was grown in YPD rich media (yeast extract 1%, peptone 2% and 2% dextrose), harvested in the logarithmic phase by centrifugation, and washed three times with ice-cold 1 M sorbitol. One to five μg of a Spe1 digested expression plasmid encoding various anti-BLyS antibody chains were mixed with competent yeast cells and electroporated using a Bio-Rad Gene Pulser XceII™ (Bio-Rad, 2000 Alfred Nobel Drive, Hercules, Calif. 94547) preset Pichia pastoris electroporation program. After one hour in recovery rich media at 24° C., the cells were plated on a minimal dextrose media (1.34% YNB, 0.0004% biotin, 2% dextrose, 1.5% agar) plate containing 300 μg/ml Zeocin and incubated at 24° C. until transformants appeared.

To screen for high titer strains, 96 transformants were inoculated in buffered glycerol-complex medium (BMGY) and grown for 72 hours followed by a 24 hour induction in buffered methanol-complex medium (BMMY). Secretion of antibody was assessed by a Protein A beads assay as follows: Fifty microliter supernatant, from 96 well plate cultures, was diluted 1:1 with 50 mM Tris pH 8.5 in a non-binding 96 well assay plate. For each 96 well plate, 2 ml of magnetic BioMag Protein A suspension beads (Qiagen, Valencia, Calif.) were placed in a tube held in a magnetic rack. After 2-3 minutes, when the beads collected to the side of the tube, the buffer was decanted off. The beads were washed three times with a volume of wash buffer equal to the original volume (100 mM Tris, 150 mM NaCl, pH 7.0) and resuspended in the same wash buffer. Twenty μl of beads were added to each well of the assay plate containing diluted samples. The plate was covered, vortexed gently and then incubated at room temperature for 1 hour, while vortexing every 15 minutes. Following incubation, the sample plate was placed on a magnetic plate inducing the beads to collect to one side of each well. On the Biomek NX Liquid Handler (Beckman Coulter, Fullerton, Calif.), the supernatant from the plate was removed to a waste container. The sample plate was then removed from the magnet and the beads were washed with 100 μl wash buffer. The plate was again placed on the magnet before the wash buffer was removed by aspiration. Twenty μl loading buffer (Invitrogen E-PAGE gel loading buffer containing 25 mM NEM (Pierce, Rockford, Ill.)) was added to each well and the plate was vortexed briefly. Following centrifugation at 500 rpm on the Beckman Allegra 6 centrifuge, the samples were incubated at 99° C. for five minutes and then run on an E-PAGE high-throughput pre-cast gel (Invitrogen, Carlsbad, Calif.). Gels were covered with gel staining solution (0.5 g Coomassie G250 Brilliant Blue, 40% MeOH, 7.5% Acetic Acid), heated in a microwave for 35 seconds, and then incubated at room temperature for 30 minutes. The gels were de-stained in distilled water overnight. Anti-BLyS mAb producing strains were selected for further DASQIP fermentation screening described in detail in Example 6.

TABLE 2
Summary of the Anti-BLyS IgG1 Fc Mutein Producing Strains.

| Anti-BLyS mAbs | Strain Name | Parent Strain |
|---|---|---|
| 6C10 | YGLY35604 | YGLY28423 |
| 10C11 | YGLY35605 | YGLY28423 |
| 6A11 | YGLY35570 | YGLY28423 |
| 5B7 | YGLY35606 | YGLY28423 |
| 5B7 | YGLY35607 | YGLY28423 |
| 1B7 | YGLY35609 | YGLY28423 |
| 5E12 | YGLY35610 | YGLY28423 |
| 5G10 | YGLY35574 | YGLY28423 |
| 8D12 | YGLY35575 | YGLY28423 |
| 2C5 | YGLY35675 | YGLY28423 |
| 2C5 | YGLY35676 | YGLY28423 |
| 6F5 | YGLY35677 | YGLY28423 |
| 6F5 | YGLY35678 | YGLY28423 |
| 8A12 | YGLY35612 | YGLY28423 |
| 8A12 | YGLY35613 | YGLY28423 |
| 1B5 | YGLY35679 | YGLY28423 |
| 1B5 | YGLY35680 | YGLY28423 |
| 10B1 | YGLY36724 | YGLY28423 |
| 10B1 | YGLY36725 | YGLY28423 |
| 4A2 | YGLY36726 | YGLY28423 |
| 4A2 | YGLY36727 | YGLY28423 |
| 14F11 | YGLY36728 | YGLY28423 |
| 14F11 | YGLY36729 | YGLY28423 |
| 9F8 | YGLY35608 | YGLY28423 |
| 9F8 | YGLY35611 | YGLY28423 |
| 5D10 | YGLY36730 | YGLY28423 |
| 1D6 | YGLY35614 | YGLY28423 |
| 1D6 | YGLY35615 | YGLY28423 |

### Example 4

### One Liter Bioreactor Anti-BLyS Monoclonal Antibody Fermentation

Antibody clones were grown in fermenters having one liter of culture volume according to the following conditions:

Media:

4% BSGY-M: 40 g/L glycerol, 20 g/L soytone, 10 g/L yeast extract, 11.9 g/L KH₂PO₄, 2.3 g/L K₂HPO₄, 50 g/L maltitol, 13.4 g/L YNB with ammonium sulfate without amino acids, 8 mg/L Biotin.

PTM2 Salts:

0.6 g/L CuSO₄-5H₂O, 80 mg/L NaI, 1.8 g/L MnSO₄-H₂O, 20 mg/L H₃BO₄, 6.5 g/L FeSO₄-7H₂O, 2.0 g/L ZnCl₂, 0.5 g/L CoCl₂-6H₂O, 0.2 g/L Na₂MoO₄-2H₂O, 0.2 g/L biotin, 5 mL/L H₂SO₄ (85%).

Inoculum Preparation:

The seed flasks were inoculated from yeast patches (isolated from a single colony) on agar plates into 0.1 L of 4% BSGY (no maltitol) in a 0.5-L baffled flask. Seed flasks were grown at 180 rpm and 24° C. (Innova 44, New Brunswick Scientific) for 48 hours.

Fed-Batch Fermentation:

Cultivations were done in 1-L (fedbatch-pro, DASGIP BioTools) bioreactors. Vessels were charged with 0.54 L of 0.2 μm filtered 4% BSGY media (with 4 drops/L Sigma 204 antifoam) and autoclaved at 121° C. for 45 minutes.

After sterilization and cooling, the aeration, agitation, and temperatures were set to 0.7 vvm, 580 rpm, and 24° C. respectively. The pH was adjusted to and controlled at 6.5 using 15% ammonium hydroxide.

Inoculation of a prepared bioreactor occurred aseptically with 60 mL from a seed flask. Agitation was ramped to maintain 20% dissolved oxygen (DO) saturation. After the initial glycerol charge was consumed, denoted by a sharp increase in the dissolved oxygen, a 50% w/w glycerol solution containing 5 mg/L biotin and 32.3 mg/L PMTi4 was triggered to feed at 7.7 g/L-h for 8 hours. During the glycerol fed-batch phase, 0.375 mL of PTM2 salts were injected manually.

Induction Phase:

After completion of the glycerol fed-batch phase, the agitation rate was locked at 580 rpm and a bolus addition of 5.4 g of methanol containing 5 mg/L biotin and 12.5 mL/L PTM2 salts was added. During methanol induction phase the, DO remained near 0% until the methanol bolus was entirely consumed. Once the DO increased to >30%, another 5.4 g bolus of the methanol feed solution was added to prolong the induction time. After methanol adaptation, it typically takes 8-9 hours to consume the 1% methanol boluses. Injections of 0.25 mL of 1.9 mg/ml PMTi4 (in methanol) were added each 24 hours of induction time.

Harvest:

Individual fermentations were harvested after 60-100 hours of induction depending upon the durability of the strain. The culture broth was clarified by centrifugation (Sorvall Evolution RC, Thermo Scientific) at 8500 rpm for 40 minutes.

### Example 5

### Anti-BLyS 5B7 15 Liter Bioreactor Cultivation

Cultivations were performed in 15 liter glass bioreactors (Applikon, Foster City, Calif.). Two 2.8 L baffled seed flasks containing 400 mL of BYS media with 4% (w/v) Glycerol at pH 6.5 were inoculated with frozen stock vials at a 1% volumetric ratio. The flasks were incubated at 24.0±0.5° C. and 180 RPM for 44±4 hours to obtain an OD600 of 25±15 absorbance units, ensuring exponential growth when the cells were transferred to the bioreactor at a 10% volumetric ratio. The bioreactor contained 8.0 L of BYSM media with temperature controlled at 24.0±0.5° C., pH controlled at 6.45±0.05 with 14% ammonium hydroxide, and dissolved oxygen (DO) maintained at 20% of saturation at atmospheric pressure and 24.0±0.5° C. by fixing the airflow rate at 0.7 vvm and cascading agitation. Depletion of the initial 40 g L⁻¹ glycerol was detected by a rapid decrease in the oxygen uptake rate (OUR measured in mmol L⁻¹ h⁻¹) and was followed by a flat glycerol feed at 7.5 g L⁻¹ h⁻¹. After completion of the glycerol fed-batch phase, methanol induction phase was initiated. The DO cascade was turned off and agitation was set to a pre-determined value to achieve OUR of 30-35 mmol L⁻¹ h⁻¹ during methanol induction phase. After DO decreased to less than 1%, the first 1% (w/v) bolus shot of methanol was delivered. All subsequent methanol shots were triggered by rapid increases in DO indicating methanol depletion.

### Example 6

### Antibody Purification and De-Sialylation of Anti-BLyS Monoclonal Antibody to Create Asialylated Forms

Purification of secreted antibody can be performed by one of ordinary skill in the art using available published methods, for example Li et al., Nat. Biotech. 24(2):210-215 (2006), in which antibodies are captured from the fermentation supernatant by Protein A affinity chromatography and further purified using hydrophobic interaction chromatography with a phenyl sepharose fast flow resin. Anti-BLyS monoclonal antibody double mutein (F243A/V264A), e.g., of 5B7 and other antibodies herein, was purified through affinity chromatography employing MabSelect resin from GE Healthcare (Cat. No. 17-5199-03). The cell free supernatant medium was loaded on to MabSelect column pre-equilibrated with 3 column volume of 20 mM Tris-HCl pH 7.0. The column was washed with 3 column volume of 20 mM Tris-HCl pH 7.0 followed by 5 column volume of 20 mM Tris-HCl, 1M NaCl pH 7.0 to remove the host cell protein contaminants. The anti-BLyS monoclonal antibody double mutein protein was eluted with 7 column volume of 50 mM sodium citrate pH 3.0. The eluted protein was neutralized immediately with 1M Tris-HCl pH 8.0. Protein A purified anti-BLyS antibodies were quantitated by Bradford method, and the titer of fermentation were calculated by dividing the volume of fermentation broth. Table 3 shows some anti-BLyS antibodies titer. The MabSelect pool sample was subsequently purified through cation exchange chromatographic step employing SP sepharose high performance resin from GE Healthcare (Cat. No. 17-1087-03). The column was equilibrated with 3 column volumes of 20 mM Sodium acetate pH 5.0 and the MabSelect pool sample that was buffer exchanged into the equilibration buffer was loaded on to the column. After loading, the column was washed with 3 column volume of the equilibration buffer and elution was performed by developing a gradient over 20 column volumes ranging from 0 to 500 mM sodium chloride. The protein that elutes around 130-250 mM sodium chloride was pooled together and subsequently formulated into 6.25 mM sodium phosphate monobasic, 10.75 mM sodium phosphate dibasic, 105.5 mM sodium chloride, 1.03 mM sodium citrate, 65.9 mM mannitol, 0.1% polysorbate 80 pH 6.0±0.2 to generate the sialylated form. A portion of the SP sepharose HP pool sample was further treated in vitro with neuraminidase enzyme (New England BioLabs Inc. Cat. No. P0720L) to remove the terminal sialic acid and further purified through an affinity chromatographic step employing MabSelect resin to remove the enzyme and subsequently formulated to create the asialylated form. Both the formulated samples were concentrated and sterile filtered through 0.2 μm filter and stored @4° C. until release.

TABLE 3
Titer of Anti-BLyS Monoclonal Antibodies in DasQip Fermentation.

| | | Antibody |
|---|---|---|
| | | Titer |
| | Anti-BLyS | (supernatant) |
| No. | antibody | mg/L |
| 1 | 6C10 | 104 |
| 2 | 10C11 | 26 |
| 3 | 6A11 | 207 |
| 4 | 5B7 | 188 |
| 5 | 1B7 | 54 |
| 6 | 5E12 | 160 |
| 7 | 5G10 | 307 |
| 8 | 8D12 | 100 |
| 9 | 9F8 | 89 |
| 10 | 8A12 | 356 |
| 11 | 1D6 | 166 |
| 12 | 2C5 | 293 |
| 13 | 6F5 | 191 |
| 14 | 1B5 | 151 |

### Example 7

### SDS-PAGE Analysis, Q-TOF Mass Spectrometry and N-Linked Glycan Analysis by HPLC

Anti-BLyS mAbs were analyzed by SDS-PAGE, Q-TOF (Quadrupole time-of-flight mass spectrometry) and 2-AB for N-linked glycan profile as part of quality control. SDS-PAGE and 2-AB assay for glycan quantitation were described in Choi et al., Proc. Natl. Acad. Sci. USA 100: 5022-5027 (2003) and Hamilton et al., Science 313: 1441-1443 (2006). To analyze the quality of and quantify the relative amount of each glycoform, the N-glycosidase F released glycans were labeled with 2-aminobenzidine (2-AB) and analyzed by HPLC. Table 4 includes some of the selected clones' glycan profiles.

To examine the intactness of anti-BLyS mAbs and their N-linked glycosylation, antibodies were analyzed by Q-TOF. In brief, 5 ul (1 mg/ml) was analyzed on an Agilent Q-TOF 6520 mass spectrometer. The dual ESI ion source was set as follows: gas temp at 350° C.; drying gas at 13 L/min; nebulizer at 45 psig; fragmentor at 150 V; skimmer at 65 V; Oct1 RF VPP at 750 V; Vcap at 3500 V. Data were analyzed using MassHunter software.

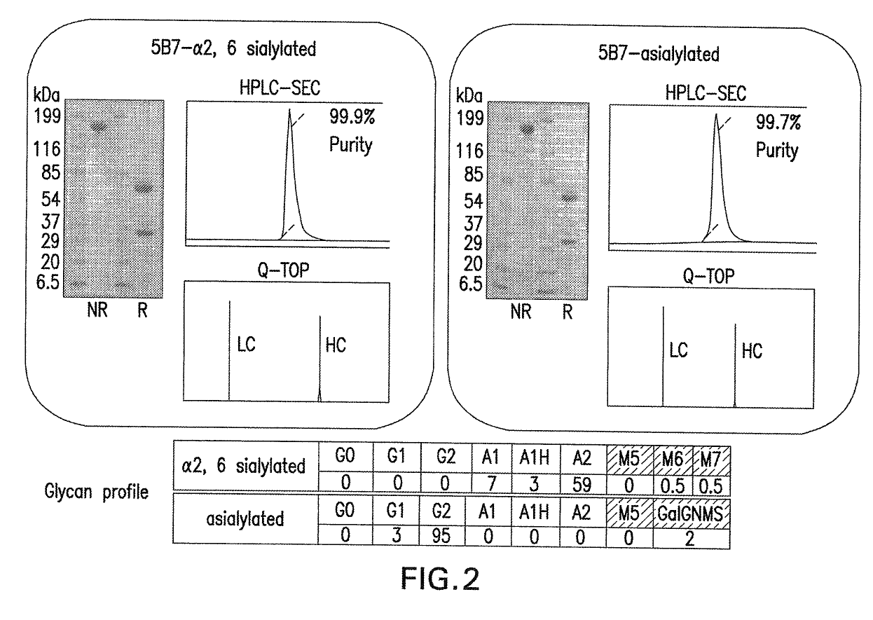

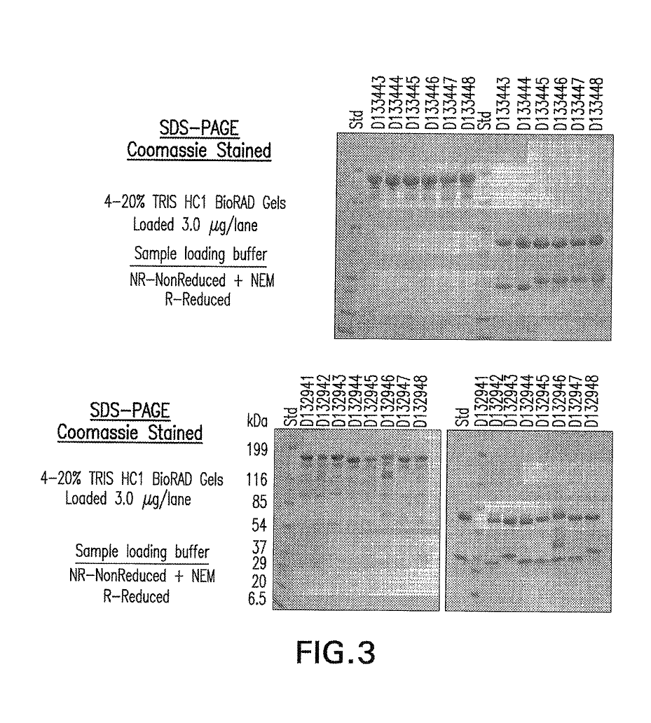

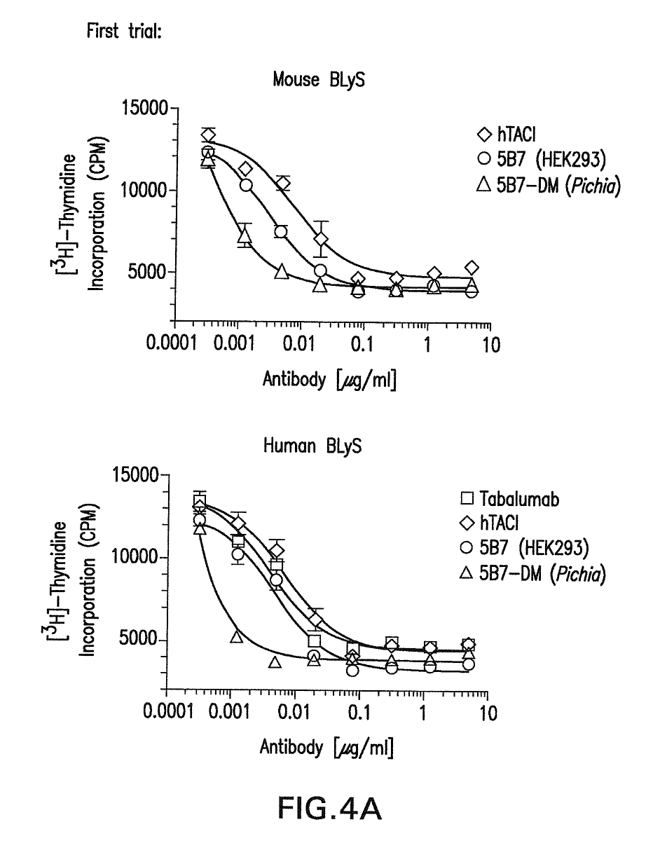

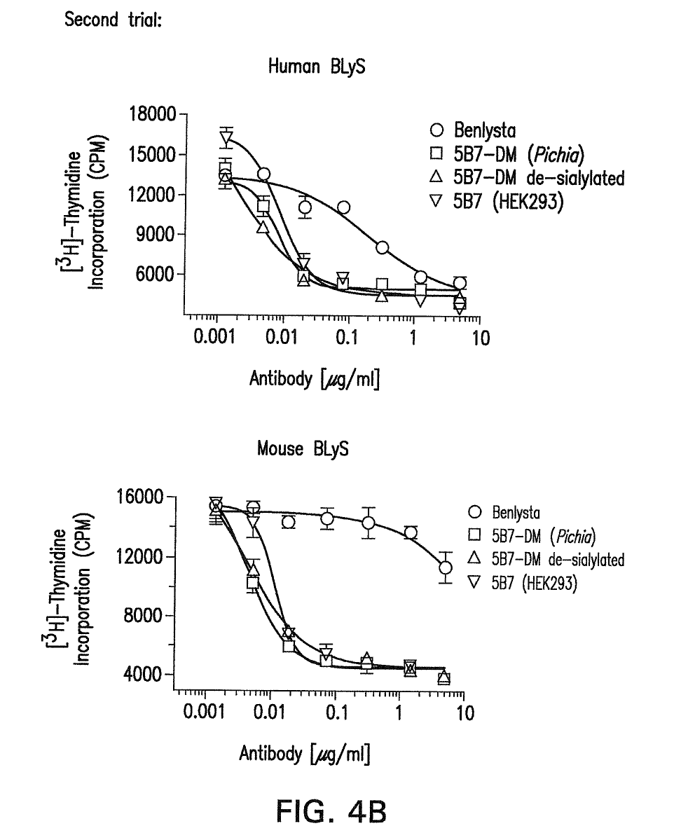

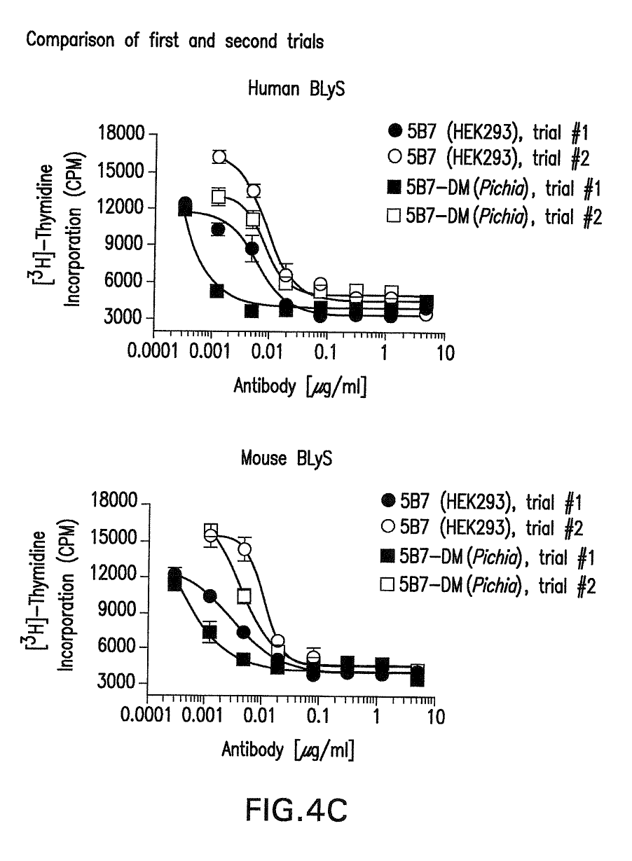

The data SDS-PAGE, Q-TOF and HPLC-SEC data generated with α2,6-sialylated and asialylated antibody 5B7 is set forth in FIG. 2. SDS-PAGE analysis of various antibodies under reducing and non-reducing conditions is set forth in FIG. 3. Gel data for D133337-D133342 are not shown.

TABLE 4
Glycan Profile of Selected α2,6-sialylated Anti-BLyS Clones.

| ID | Strain | Ab | G0 | G1 | G2 | A1 | A1H | A2 | M4 | M5 | M6 | M7 | M8 |
|---|---|---|---|---|---|---|---|---|---|---|---|---|---|
| D133337 | YGLY35604 | 6C10 | 0 | 0 | 0 | 11 | 7 | 77 | 0 | 3 | 1 | 1 | 0 |
| D133338 | YGLY35605 | 10C11 | 0 | 0 | 0 | 9 | 12 | 65 | 0 | 4 | 5 | 2 | 1 |
| D133339 | YGLY35606 | 5B7 | 0 | 0 | 0 | 12 | 12 | 71 | 1 | 2 | 1 | 0 | 0 |
| D133340 | YGLY35607 | 5B7 | 0 | 0 | 0 | 12 | 12 | 73 | 0 | 1 | 1 | 0 | 0 |
| D133341 | YGLY35608 | 9F8 | 0 | 0 | 0 | 12 | 12 | 73 | 0 | 0 | 1 | 1 | 0 |
| D133342 | YGLY35609 | 1B7 | 0 | 0 | 0 | 16 | 11 | 62 | 0 | 7 | 2 | 1 | 0 |
| D133343 | YGLY35610 | 5E12 | 0 | 0 | 1 | 12 | 9 | 75 | 0 | 1 | 0 | 0 | 0 |
| D133344 | YGLY35611 | 9F8 | 1 | 2 | 1 | 5 | 20 | 69 | 0 | 1 | 1 | 1 | 0 |
| D133345 | YGLY35612 | 8A12 | 1 | 0 | 0 | 14 | 12 | 65 | 0 | 2 | 3 | 1 | 0 |
| D133346 | YGLY35613 | 8A12 | 0 | 0 | 2 | 15 | 7 | 72 | 0 | 2 | 1 | 0 | 0 |
| D133347 | YGLY35614 | 1D6 | 1 | 1 | 2 | 21 | 13 | 53 | 0 | 3 | 3 | 2 | 0 |
| D133348 | YGLY35615 | 1D6 | 0 | 0 | 1 | 17 | 17 | 55 | 1 | 5 | 3 | 1 | 0 |
| D132941 | YGLY35568 | 6C10 | | | | 7 | 5 | 87 | | | | | |
| D132942 | YGLY35569 | 10C11 | | | | 10 | 12 | 71 | | 3 | 3 | 1 | |
| D132943 | YGLY35570 | 6A11 | | | 1 | 16 | 14 | 65 | 1 | 2 | 0 | | |
| D132944 | YGLY35571 | 5B7 | | | 1 | 12 | 7 | 78 | 1 | 1 | | | |
| D132945 | YGLY35572 | 1B7 | | | | 14 | 14 | 62 | 1 | 6 | 4 | 1 | |
| D132946 | YGLY35573 | 5E12 | | | | 9 | 10 | 79 | 0 | 1 | 0 | | |
| D132947 | YGLY35574 | 5G10 | | | 1 | 16 | 14 | 62 | 1 | 4 | 1 | 0 | |
| D132948 | YGLY35575 | 8D12 | | | 1 | 14 | 11 | 71 | 1 | 2 | 0 | | |

A1 indicates an N-glycan with 1 sialic acid.
A1H indicates an N-glycan with 1 sialic acid along with mannose.
A2 indicates an N-glycan with 2 sialic acids

### Example 8

### Human and Mouse BLyS Antigen Binding Assay by Biacore T100

A Series S CM5 Chip (GE Healthcare) was amine coupled with an anti-human Fc capturing antibody (GE Healthcare Human Fc Kit) to >10,000 RUs. Sample antibodies were diluted in 1×HBS-EP+ running buffer to 0.15 ug/mL and injected for 45 seconds at 10 uL/min. Serially diluted human or mouse BLyS (R&D Systems) was injected for 6 minutes at 30 μl/min and dissociation was monitored for 20 minutes. The anti-BLyS and BLyS complex were removed from the capture surface with 3M MgCl₂ for 40 seconds at 10 μl/min. Binding data was reference subtracted with both a negative BLyS binder (commercial Herceptin) and a 0 μM BLyS injection. Biacore Evaluation software was used to fit the data to a 1:1 Binding Model.

TABLE 5
Human and Mouse BLyS Antigen Binding Assay for Anti-BLyS
Monoclonal Antibodies.
Human BLyS

| | kₐ (×10⁵) | k_{d} (×10⁻⁵) | K_{D} | |
|---|---|---|---|---|
| | 1/M * s | 1/s | (pM) | |
| | Association | Dissociation | Dissociation | |
| Samples | Rate | Rate | Constant | Comment |
| belimumab | 5.06 | 8.55 | 167 | n = 2 |
| hTACI | 3.12 | 8.34 | 268 | R&D Systems |
| mTACI | 3.97 | 23.9 | 602 | R&D Systems |
| 6C10 | 3.68 | 2.5 | 68 | D132941 |
| 10C11 | 8.55 | 8.4 | 98 | D132942 |
| 6A11 | 1.92 | 17.4 | 905 | D132943 |
| 5B7 | 6.78 | 4.6 | 68 | D132944 |
| 1B7 | 14.2 | 9.61 | 64 | D132945 |
| 5E12 | 2.84 | 17.8 | 628 | D132946 |
| 5G10 | 2.17 | 22.6 | 1040 | D132947 |
| 8D12 | 11.1 | 5.4 | 49 | D132948 |

Mouse BLyS

| | kₐ (×10⁵) | k_{d} (×10⁻⁴) | K_{D} | |
|---|---|---|---|---|
| | 1/M * s | 1/s | (pM) | |
| | Association | Dissociation | Dissociation | |
| Samples | Rate | Rate | Constant | Comment |
| belimumab | 19.85 | 1.25 | 63.5 | n = 2 |
| hTACI | 6.21 | 0.65 | 105 | |
| mTACI | 6.82 | 1.21 | 177 | |
| 6C10 | 9.67 | 2.24 | 232 | D132941 |
| 10C11 | 20.6 | 2.75 | 134 | D132942 |
| 6A11 | 14.7 | 1.77 | 121 | D132943 |
| 5B7 | 27.3 | 1.02 | 37 | D132944 |
| 1B7 | 29.8 | 1.71 | 57 | D132945 |
| 5E12 | 6.72 | 1.08 | 121 | D132946 |
| 5G10 | 6.16 | 4.51 | 732 | D132947 |
| 8D12 | 14.6 | 1.96 | 134 | D132948 |

### Example 9

### Mouse Splenic B-Cell Proliferation Assay

Spleens were extracted from female Balb/cJ mice, and B-cells were isolated using MACS purification (Miltenyi 130-090-862). 105 purified B-cells per well were plated into 96 well round bottom plates (Falcon 353077) in complete DMEM medium supplemented with 10% FCS, HEPES, L-glutamine, sodium pyruvate, penicillin/streptomycin, and 2-ME. Cells were stimulated with 2.5 μg/ml soluble anti-IgM (Jackson 115-006-020) and 5 ng/ml of either soluble recombinant mouse or human BAFF (BLyS) (R&D Systems, 2106-BF-010 or 2149-BF-010). Belimumab, Tabalumab, human TACI (immunoglobulin fusion), or the 5B7 antibodies were titrated at the indicated concentrations. Cells were cultured for a total of 72 hours in a 37° C. incubator and 5% CO₂, with the final 24 hours in 1 μCi per well of ³H-thymidine. Cells were harvested and total CPM were counted.

TABLE 6
Assay of Anti-BLyS Antibodies for Inhibition of Mouse B-cell
Proliferation.

| | IC₅₀ | IC₅₀ |
|---|---|---|
| | hBLyS | mBLyS |
| | stimulated | stimulated |
| Antibody | (ng/ml) | (ng/ml) |
| belimumab | 18 | NA |
| tabalumab | 4 | NA |
| hTACI-Ig | 7 | 7 |
| 5B7-DM Pichia | ≦1 | ≦1 |
| 5B7-HEK293 | 4 | 3 |

5B7-DM Pichia: Pichia expressed double mutein 5B7 antibody.
5B7-HEK293: HEK293 cell expressed 5B7 antibody.
NA: no activity

### Example 10

### Efficacy of Anti-BLyS Antibodies in a Lupus Animal Model

Well-established animal models are available to test the in vivo efficacy of BLyS blockers which could be soluble receptors, antagonist peptide Fc fusions, or mAbs. In particular, BLyS blockers can be tested in vivo in a number of animal models of autoimmune disease, such as MRL-lpr/lpr or NZBxNZW F1 congenic mouse strains which serve as a model of SLE (systemic lupus erythematosus). Such animal models are known in the art.

Offspring of a cross between New Zealand Black (NZB) and New Zealand White (NZW) mice develop a spontaneous form of SLE that closely resembles SLE in humans. The offspring mice, known as NZB/WF1, begin to develop IgM autoantibodies against T-cells at 1 month of age, and by 5-7 months of age, Ig anti-DNA autoantibodies are the dominant immunoglobulin. Polyclonal B-cell hyperactivity leads to overproduction of autoantibodies. The deposition of these autoantibodies, particularly ones directed against single stranded DNA, is associated with the development of glomerulonephritis, which manifests clinically as proteinuria, azotemia, and death from renal failure. Kidney failure is the leading cause of death in mice affected by spontaneous SLE, and in the NZB/WF1 strain, this process is chronic and obliterative. The disease is more rapid and severe in females than males, with mean survival of only 245 days as compared to 406 days for the males. The immune nephritis seen in the NZBW mice is very similar to the glomerulonephritis seen in human SLE, making this spontaneous murine model useful for testing of potential SLE therapeutics.

In order to assess the effect of BLyS blockers on lupus development in NZB/WF1 mice, TACI-Ig shows a dose-dependent delay in SLE onset. In addition, alpha 2,6 sialylated TACI-Ig is compared with asialylated TACI-Ig in three different dosages in a prophylactic setting. Briefly, 10 animals of each group are dosed three times a week by i.p. injection at 1 mpk, 5 mpk and 50 mpk. Alpha 2,6-TACI-Ig shows a trend towards increased efficacy compared to TACI-Ig at all three doses. Finally, there is a trend towards increased half-life in vivo of the alpha2,6-TACI-Ig vs. TACI-Ig. Both forms of TACI show a dose-dependent reduction in circulating B cells.

In order to assess the efficacy of the de novo anti-BLyS antibodies of the present invention, those which can cross-react to mouse BLyS in an in vitro B-cell proliferation assay will be tested in NZB/WF1 models. The variable region will be formulated into an IgG1 format with two mutations at the Fc region (F243A/V264A) which allows adding sialic acid at its N-linked glycans. The full length antibodies will be expressed in the GlycoEngineered Pichia host which is capable of adding alpha-2,6 sialic acid at its glycan. Material will be generated from these production cell lines. At the same time, asialylated anti-BLyS antibodies with the identical amino acid sequence will be produced, but without sialic acid at its glycan by treatment of the sialylated anti-BLyS antibodies with neuraminidase. NZB/WF1 animal will be dosed with these two forms in various dosages which are similar to the TACI-Ig study described above in a prophylactic setting. Disease progression will be monitored by proteinuria, autoantibodies, and serological markers.

### Example 11

### APRIL Biacore Binding

Recombinant protein NG (Thermo Scientific, Cat #21186) was diluted to 100 μg/ml in 10 mM Sodium Acetate pH 4.0 and then immobilized on the surface of a pre-activated Series S CM4 Biacore chip (GE, Cat #BR-1005-34) using amine coupling. The monoclonal antibodies were captured individually on each of the four CM4 chip flow cells (with flow cell one serving as a reference) by injecting about 5 μg/ml monoclonal antibodies over individual flow cells for 120 seconds at 10 μl/min (yields 1,000-2,000 RU capture). To determine binding of human APRIL (R&D Systems, Cat #5860-AP-010/CF) and mouse APRIL (R&D Systems, Cat #7907-AP-010/CF) to the anti-BLyS antibodies, the APRIL protein as injected manually at 50 nM for 300 seconds at 10 μl/min. Binding response was measured as the difference in total RU increase during 300 second APRIL injection of each individual flow cell and the reference flow cell (e.g., Flow cell 2-1).

TABLE 7
ARPIL Biacore binding data.

| Mab | Source | hAPRIL | mAPRIL |
|---|---|---|---|
| 6C10 | Pichia | NO | NO |
| | HEK293 | NO | NO |
| 1B7 | Pichia | NO | NO |
| | HEK293 | NO | NO |
| 10C11 | Pichia | NO | YES |
| | HEK293 | NO | YES |
| 8D12 | Pichia | NO | NO |
| | HEK293 | NO | NO |
| 5B7 | Pichia | NO | NO |
| | HEK293 | NO | NO |
| TACI rec | Human | YES | YES |
| TACI rec | Mouse | YES | YES |

### Example 12

### Binding and Receptor Blocking

In this example, the ability of various anti-BLyS antibodies to bind to BLyS and to block binding of BLyS to BR3, BCMA or TACI was determined.

### Methods and Materials: Biacore Kinetic Studies

Recombinant protein NG (Thermo Scientific, Cat #21186) was diluted to 100 μg/ml in 10 mM sodium acetate pH 4.0 and then immobilized on the surface of a pre-activated Series S CM5 Biacore chip (GE, Cat #BR-1005-34) using amine coupling. To determine binding kinetics of anti-BLyS antibodies to human BLyS (R&D systems, Cat #2149-BF-010/CF) or mouse BLyS (R&D Systems, Cat #2106-BF-010/CF), the mAbs were captured on each of the four CM5 chip flow cells (with flow cell one serving as a control and reference) by injecting 0.5 μg/ml Abs over individual flow cells for 30 seconds at 10 μl/min (yields ˜100 RU capture). hBLyS or mBLyS was then passed over all four flow cells at 30-50 μl/min for 600 seconds, followed by a dissociation period of 2,400 seconds. After the dissociation period, the chip was regenerated back to baseline by injecting 10 mM glycine pH 1.5 for 120 seconds over the four flow cells. This was repeated for each BLyS injection over 11 injections of a 2/3-fold dilution titration range starting at 20 nM. The entire data set was fit to a 1:1 interaction model to give kₒₙ, k_{off}, and K_{d} values. The assay was run at 25° C. in 1× biacore running buffer HBS-EP+ (GE, Cat #BR-1006-69).

TABLE 8
Biacore Binding analysis.

| | | BIACORE KINETICS | | | | | |
|---|---|---|---|---|---|---|---|
| | | ka (1/Ms) E+05 | | kd (1/s) | | K_{D} (pM) | |
| Ab clone | Source | hBLyS | mBLyS | hBLyS | mBLyS | hBLyS | mBLyS |
| 5B7 | HEK293 | 1.9E+05 | 6.7E+05 | 4.2E−05 | 4.2E−05 | 224 | 62 |
| 5B7-DM | Pichia | 1.9E+05 | 6.2E+05 | 3.5E−05 | 4.0E−05 | 179 | 65 |
| 1B7 | HEK293 | 3.1E+05 | 4.9E+05 | 5.3E−05 | 5.0E−05 | 168 | 103 |
| 1B7-DM | Pichia | 2.9E+05 | 4.7E+05 | 3.1E−05 | 3.8E−05 | 109 | 82 |
| 6C10 | HEK293 | 1.2E+05 | 2.0E+05 | 5.0E−05 | 6.7E−05 | 434 | 334 |
| 6C10-DM | Pichia | 1.3E+05 | 3.3E+05 | 5.5E−05 | 7.9E−05 | 421 | 234 |
| 10C11 | HEK293 | 2.2E+05 | 4.3E+05 | 4.4E−05 | 6.5E−05 | 201 | 153 |
| 10C11-DM | Pichia | 2.2E+05 | 4.3E+05 | 4.3E−05 | 6.6E−05 | 194 | 154 |
| 8D12 | HEK293 | | | | | | |
| 8D12-DM | Pichia | 2.6E+05 | 5.8E+05 | 3.5E−05 | 5.7E−05 | 138 | 99 |

### Methods and Materials: DELFIA Receptor Blocking Studies

Anti-BLyS mAb receptor blocking assay was evaluated using Perkin Elmer's DELFIA assay technology (dissociation-enhanced lanthanide fluorescence immunoassay) which is a time-resolved fluorescence (TRF) intensity technology. Custom europium labeled human BLyS (R&D systems, Cat #2149-BF-010/CF) and mouse BLyS (R&D Systems, Cat #2106-BF-010/CF) were generated by Perkin Elmer to a final concentration of hBLyS of 16.8 μM (0.2 Ue: 1 protein) and mBLyS 5.079 μM (1.14 Eu: 1 protein). DELFIA yellow 96-well low auto-fluorescent assay plates (PE, Cat #AAAND-0001) were coated overnight at 4° C. with 0.1 μg/well of select human or mouse BLyS cognate receptor chimera from R&D Systems (BCMA, BR3 and TACI). After 5× washing with 200 ul/well of DELFIA 1× wash solution (PE, Cat #4010-0010), plates were blocked with 3% BSA purified from trace heavy metal amounts (PE, Cat #CR84-100) for 3 hours with shaking at room temperature. Plates were washed again five times at 200 μl/well with DELFIA wash solution.

After wash, 50 μl/well of BLyS mAbs were added to wells across the plate in a 9 point 1/3-fold dilution titration series starting at 2,000 pM to 0.3 pM. The final 3 wells across the plate served as controls (2 wells contained 2,000 pM mAbs for negative and background controls while the third contained no mAbs for positive control). 50 ul/well of 100 pM BLyS was then added to each well, except the negative-control wells, and plates were briefly spun down. After 18-24 hrs at 4° C., plates were read on PE EnVision plate reader with laser excitation, LANCE/DELFIA top mirror, and emission filter Eu-615 with a delay of 400 μs. Data were fit to four parameter (variable slope) inhibition dose response curve using GraphPad PRISM 5.0 software.

TABLE 9
Receptor Blocking Analysis.

| | BR3 | | BCMA | | TACI | | Biacore | |
|---|---|---|---|---|---|---|---|---|
| | IC50 (pM) | | IC50 (pM) | | IC50 (pM) | | KD (pM) | |
| Antibody | hBLyS | mBLyS | hBLyS | mBLyS | hBLyS | mBLyS | hBLyS | mBLyS |
| 10C11 (293) | 33.5 | >2,000 | <0.3 | 886 | <0.3 | 732 | 224 | 62 |
| 10C11-DM (Pichia) | 41 | >2,000 | 1.2 | >2,000 | 1.2 | >2,000 | 179 | 65 |
| 1B7 (293) | 20.6 | >2,000 | <0.3 | >2,000 | <0.3 | >2,000 | 168 | 103 |
| 1B7-DM (Pichia) | 22.2 | >2,000 | <0.3 | >2,000 | <0.3 | >2,000 | 109 | 82 |
| 5B7 (293) | 52.6 | 106.7 | 1.8 | 1.3 | <0.3 | <0.3 | 434 | 334 |
| 5B7-DM (Pichia) | 37.7 | 94.01 | 1.6 | <0.3 | 0.51 | <0.3 | 421 | 234 |
| 6C10 (293) | 121 | 783.6 | 10.6 | 98 | 0.67 | 679 | 201 | 153 |
| 6C10-DM (Pichia) | 87.3 | >2,000 | 17 | >2,000 | 0.74 | 445 | 194 | 154 |
| 8D12 (293) | 62.2 | >2,000 | 630 | >2,000 | <0.3 | >2,000 | | |
| 8D12-DM (Pichia) | 101 | >2,000 | 1281 | >2,000 | <0.3 | >2,000 | 138 | 99 |
| Tabalumab | 89.9 | >2,000 | — | — | — | — | | |
| Benlysta | | >2,000 | — | — | — | — | | |
| AMG623 | 9.3 | 147.2 | <0.3 | 11.5 | <0.3 | <0.3 | | |
| TACI-Ig | 159 | 515 | 112 | 784 | 12.75 | 15.8 | | |

The present invention is not to be limited in scope by the specific embodiments described herein. Indeed, the scope of the present invention includes embodiments specifically set forth herein and other embodiments not specifically set forth herein; the embodiments specifically set forth herein are not necessarily intended to be exhaustive. Various modifications of the invention in addition to those described herein will become apparent to those skilled in the art from the foregoing description. Such modifications are intended to fall within the scope of the claims.

Patents, patent applications, publications, product descriptions, and protocols are cited throughout this application, the disclosures of which are incorporated herein by reference in their entireties for all purposes.

1. An isolated antibody or antigen-binding fragment thereof that binds specifically to BLyS:
(1)
comprising a light chain immunoglobulin that comprises:
CDR-L1 comprising an amino acid sequence having at least 80% amino acid sequence identity or similarity to an amino acid sequence selected from the group consisting of SEQ ID NOs: 96-114;
CDR-L2 comprising an amino acid sequence having at least 80% amino acid sequence identity or similarity to an amino acid sequence selected from the group consisting of SEQ ID NOs: 115-133; and/or
CDR-L3 comprising an amino acid sequence having at least 80% amino acid sequence identity or similarity to an amino acid sequence selected from the group consisting of SEQ ID NOs: 134-152; and/or
a heavy chain immunoglobulin that comprises:
CDR-H1 comprising an amino acid sequence having at least 80% amino acid sequence identity or similarity to an amino acid sequence selected from the group consisting of SEQ ID NOs: 39-57;
CDR-H2 comprising an amino acid sequence having at least 80% amino acid sequence identity or similarity to an amino acid sequence selected from the group consisting of SEQ ID NOs: 58-76; and/or
CDR-H3 comprising an amino acid sequence having at least 80% amino acid sequence identity or similarity to an amino acid sequence selected from the group consisting of SEQ ID NOs: 77-95; and/or
(2)
comprising an immunoglobulin light chain that comprises a variable region comprising an amino acid sequence having at least 80% amino acid sequence identity or similarity to the variable region of an immunoglobulin comprising an amino acid sequence selected from the group consisting of SEQ ID NOs: 20-38; and/or an immunoglobulin heavy chain that comprises a variable region comprising an amino acid sequence having at least 80% amino acid sequence identity or similarity to the variable region of an immunoglobulin comprising an amino acid sequence selected from the group consisting of SEQ ID NOs: 1-19; and/or
(3)
that cross-blocks, is cross-blocked by or binds the same epitope as any foregoing antibody or antigen-binding fragment; wherein, optionally, said antibody or fragment comprises one or more N-linked glycans represented by the structure:

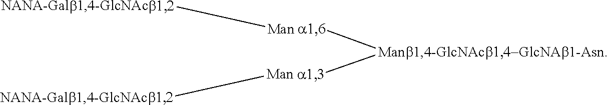

2. The antibody or fragment of claim 1 which is a a fully human antibody, monoclonal antibody, a labeled antibody, a bivalent antibody, a polyclonal antibody, a bispecific antibody, a chimeric antibody, a recombinant antibody, an anti-idiotypic antibody, a humanized antibody or a bispecific antibody, camelized single domain antibody, a diabody, an scfv, an scfv dimer, a dsfv, a (dsfv)₂, a dsFv-dsfv′, a bispecific ds diabody, an Fv, an Fab, an Fab′, an F(ab′)₂, or a domain antibody.

3. The antibody or antigen-binding fragment of claim 1 linked to a gamma immunoglobulin constant domain optionally comprising one or more mutations selected from the group consisting of F243X, V264X, S267X and L328X.

4. The antibody or antigen-binding fragment of claim 1 which is glycosylated with the N-linked glycan: SA₍₁₋₄₎Gal₍₁₋₄₎GlcNAc₍₂₋₄₎Man₃GlcNAc₂.

5. An injection device or vessel comprising the antibody or fragment of claim 1.

6. An isolated host cell comprising the antibody or antigen-binding fragment thereof of claim 1.

7. The host cell of claim 6 which is Pichia.

8. The host cell of claim 6 comprising the genotype:
ura5Δ::ScSUC2 och1Δ::lacZ bmt2Δ::lacZ/KIMNN2-2
mnn4L1Δ::lacZ/MmSCL35A3 pno1Δ mnn4Δ::lacZ
ADE1::lacZ/NA10/MmSLC35A3/FB8
his1Δ::lacZ/ScGAL10/XB33/DmUGT
arg1Δ::HIS1/KD53/TC54
bmt4Δ::lacZ bmt1Δ::lacZ bmt3Δ::lacZ
TRP2::ARG1/MmCST/HsGNE/HsCSS/HsSPS/MmST6-33
ste13Δ::lacZ-URA5-lacZ/TrMDS1 dap2Δ::NatR
TRP5::HygRMmCST/HsGNE/HsCSS/HsSPS/MmST6-33
att1Δ::ScARR3/LmSTT3D.

9. An isolated polypeptide that comprises an amino acid sequence having at least 80% sequence similarity or identity to an amino acid sequence selected from the group consisting of SEQ ID NOs: 1-152 or an injection device or vessel comprising the same.

10. An isolated polynucleotide that encodes a polypeptide of claim 9 or an injection device or vessel comprising the same.

11. The polynucleotide of claim 10 comprising the nucleotide sequence set forth in SEQ ID NO: 162 or 163.

12. An isolated vector comprising the polynucleotide of claim 10.

13. A pharmaceutical composition comprising the antibody or antigen-binding fragment thereof of claim 1 and a pharmaceutically acceptable carrier; or an injection device or vessel comprising the same.

14. A composition comprising the antibody or antigen-binding fragment thereof of claim 1; in association with a further chemotherapeutic agent; or an injection device or vessel comprising the same.

15. The composition of claim 14 wherein the further chemotherapeutic agent is a member selected from the group consisting of: belimumab, tadalumab, denosumab, aspirin, diclofenac, diflunisal, etodolac, fenoprofen, floctafenine, flurbiprofen, ibuprofen, indomethacin, ketoprofen, ketorolac, meclofenamate, mefenamic acid, meloxicam, nabumetone, naproxen, oxaprozin, phenylbutazone, piroxicam, salsalate, sulindac, tenoxicam, tiaprofenic acid, tolmetin, betamethasone benzoate, betamethasone valerate, clobetasol propionate, desoximetasone, fluocinolone acetonide, flurandrenolide, a topical steroid, alclometasone dipropionate, aloe vera, amcinonide, amcinonide, anthralin, betamethasone dipropionate, betamethasone valerate, calcipotriene, clobetasol propionate, coal tar, Dead Sea salts, desonide, desonide; betamethasone valerate, desoximetasone, diflorasone diacetate, epsom salts, fluocinolone acetonide, fluocinonide, flurandrenolide, fluticasone propionate, halcinonide, halobetasol propionate, hydrocortisone valerate, hydrocortisone, mometasone furoate, oilated oatmeal, petroleum jelly, prednicarbate, salicylic acid, tazarotene, triamcinolone acetonide, a mixture of hydrocortisone, dexamethasone, methylprednisolone and prednisolone, alefacept, etanercept, cyclosporine, methotrexate, acitretin, isotretinoin, hydroxyurea, mycophenolate mofetil, sulfasalazine, 6-Thioguanine, anakinra, injectable gold, penicillamine, azathioprine, chloroquine, hydroxychloroquine, sulfasalazine, oral gold, auranofin, gold sodium thiomalate, aurothioglucose, mesalamine, sulfasalazine, budesonide, metronidazole, ciprofloxacin, azathioprine, 6-mercaptopurine or dietary supplementation of calcium, folate, vitamin B12, celecoxib, rofecoxib, valdecoxib, lumiracoxib, etoricoxib, efalizumab, adalimumab, infliximab, rituximab, tocilizumab, and ABX-IL8.

16. A method for producing the antibody or antigen-binding fragment thereof of claim 1 comprising introducing a polynucleotide encoding an immunoglobulin light chain of the antibody or fragment and/or an immunoglobulin heavy chain of the antibody or fragment into a host cell; culturing the host cell under conditions favorable to expression of the chains; and, optionally, isolating the chains.

17. A method for treating or preventing a medical disorder mediated by BLyS, in a subject, comprising administering a therapeutically effective amount of an antibody or antigen-binding fragment thereof of claim 1 to the subject.

18. The method of claim 17 wherein the medical disorder is an autoimmune or inflammatory disorder.

19. The method of claim 17 wherein the medical disorder is a member selected from the group consisting of: appendicitis, peptic ulcer, gastric ulcer and duodenal ulcer, peritonitis, liver steatosis, pancreatitis, inflammatory bowel disease, colitis, ulcerative colitis, pseudomembranous colitis, acute colitis, ischemic colitis, diverticulitis, epiglottitis, achalasia, cholangitis, cholecystitis, coeliac disease, hepatitis, Crohn's disease, enteritis, Whipple's disease, asthma, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, and urethritis, bronchitis, emphysema, rhinitis, fibrosis, cystic fibrosis, pneumonitis, adult respiratory distress syndrome, pneumoultramicroscopicsilicovolcanoconiosis, alvealitis, bronchiolitis, pharyngitis, pleurisy, sinusitis, dermatitis, atopic dermatitis, dermatomyositis, sunburn, urticaria warts, wheals, stenosis, restenosis, vasulitis, angiitis, endocarditis, arteritis, atherosclerosis, thrombophlebitis, pericarditis, myocarditis, myocardial ischemia, periarteritis nodosa, rheumatic fever, meningitis, encephalitis, multiple sclerosis, neuritis, neuralgia, uveitis, arthritides and arthralgias, osteomyelitis, fasciitis, Paget's disease, gout, periodontal disease, rheumatoid arthritis (RA), synovitis, myasthenia gravis, thryoiditis, systemic lupus erythematosus (SLE), goodpasture's syndrome, behcets's syndrome, allograft rejection, graft-versus-host disease, B-cell lymphoma, non-hodgkins lymphoma, leukemia, chronic lymphocytic leukemia, granulomatosis with polyangiitis (GPA; Wegener's granulomatosis), Churg-Strauss syndrome, an ANCA-associated vasculitide and microscopic polyangiitis.

20. The method of claim 17 wherein antineutrophil cytoplasmic antibodies are in the blood of the subject.


SEQUENCE LISTING (163 sequences)

SEQ ID NO: 1 - protein, 455 aa, Homo sapiens
EVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARTYYDILTGYYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLAPPKPKDTLMISRTPEVTCVVADVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 457 aa, Homo sapiens
EVQLVQSGAEVKKPGSSVKVSCKASGGTFSSYAISWVRQAPGQGLEWMGRIIPILGIANYAQKFQGRVTITADKSTSTAYMELSRLRSDDTAVYYCARDRFYDILTGYYTYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLAPPKPKDTLMISRTPEVTCVVADVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 454 aa, Homo sapiens
QIQLVQSGTEVKKPGASVKVSCKASGYTFTDYYMHWVRQAPGQGLEWMGRINPNSGGTNYAQKFQGRVTMTRDTSISTAYMELSRLRSDDTFVYYCAREGYDFLTGYTLGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLAPPKPKDTLMISRTPEVTCVVADVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - protein, 454 aa, Homo sapiens
QVQLVQSGAEVKKPGASVKVSCKASGGTFSSYAISWVRQAPGQGLEWMGGIIPIFGTANYAQKFQGRVTITADKSTSTAYMELSSLRSEDTAVYYCARVDYDILTGYYMGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLAPPKPKDTLMISRTPEVTCVVADVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 5 - protein, 454 aa, Homo sapiens
QVQLVQSGAKVKKPGASAKVSCKASGYTFTSYAMHWVRQAPGQRLEWMGWINAGNGNTKYSQKFQGRVTITRDTSASTAYMELSSLRSEDTAVYYCARAYYDILTGYSVYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLAPPKPKDTLMISRTPEVTCVVADVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 453 aa, Homo sapiens
QVQLVQSGAEVKKPGSSVKVSCKASGGTFSSYAISWVRQAPGQGLEWMGWMNPNSGNTGYAQKFQGRVTMTRNTSISTAYMELSSLRSEDTAVYYCARGHADILTGYLDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLAPPKPKDTLMISRTPEVTCVVADVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 7 - protein, 453 aa, Homo sapiens
QMQLVQSGAEVKKPGASVKVSCKASGYTFTSYAMHWVRQAPGQRLEWMGWINAGNGNTKYSQKFQGRVTITRDTSASTAYMELSSLRSEDTAVYYCAREMAVAGTYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLAPPKPKDTLMISRTPEVTCVVADVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 8 - protein, 454 aa, Homo sapiens
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCARAYYDILTGYSVYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLAPPKPKDTLMISRTPEVTCVVADVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - protein, 454 aa, Homo sapiens
QVQLVQSGAEVKKPGASVKVSCKASGYTFTGYYIHWVRQAPGQGLEWMGWINPSSGGTNYAQKFQGRVTMTRDTSASTAFMSLSSLRSEDTAVYYCARSYYDILTGYSRNPFDNWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLAPPKPKDTLMISRTPEVTCVVADVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 459 aa, Homo sapiens
EVQLVQSGAEVKKPGSSVKVSCKASGGTFSSYAISWVRQAPGQGLEWMGGISAYKGNTNYAQKLQGRVTMTTDTSTSTAYMELRSLRSEDTAVYYCARGRLAEYDILTGYYFNTWAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLAPPKPKDTLMISRTPEVTCVVADVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 458 aa, Homo sapiens
QVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAREGQGYDILTGYYTRGYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLAPPKPKDTLMISRTPEVTCVVADVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 459 aa, Homo sapiens
QVQLQESGPGLVEPSETLSLTCTVSGGSISGRNYYWGWIRQSTGKGLEWIGSIYYSGSTYYNPSLKSRVTISVDTSKNQFSLKLSSVTAADTAVYYCARDLANYDILTGYYRARGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLAPPKPKDTLMISRTPEVTCVVADVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - protein, 454 aa, Homo sapiens
EVQLVESGAEVKKPGASVKVSCKASGYTFTSYGISWVRQAPGQGLEWMGWISAYNGNTNYAQKLQGRVTMTTDTSTSTAYMELRSLRSDDTAVYYCARADYDILTGYPRGPVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLAPPKPKDTLMISRTPEVTCVVADVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 459 aa, Homo sapiens
QVQLVQSGGGVVQPGRSLRLSCAASGFTFSSYGMHWVRQAPGKGLEWVAVISHDGSNKYYADSVKGRFTISRDNSKNTLYLQMNSLRAEDTAVYYCAKDRSYYDILTGYYIPYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLAPPKPKDTLMISRTPEVTCVVADVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 454 aa, Homo sapiens
QVQLVQSGAEVKKPGSSVKVSCKASGGTFSSYAISWVRQAPGQGLEWMGRINPNSGGTNYAQKFQGRVTMTRDTSISTAYMELSRLRSDDTFVYYCAREGYDFLTGYTLGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLAPPKPKDTLMISRTPEVTCVVADVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 457 aa, Homo sapiens
QVQLQQSGAEVKKPGASVKVSCKASGYTFTSYYMHWVRQAPGQGLEWMGWINAGNGNTKYSQKFQGRVTITRDTSASTAYMELSSLRSEDTAVYYCARDNLSYDILTGYSFGGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLAPPKPKDTLMISRTPEVTCVVADVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 453 aa, Homo sapiens
QIQLVQSGPEVKKPGASVKVSCKASGYTFTGYYMHWVRQAPGQGLEWMGWINPNSGGTNYAQKFQGRVTMTRDTSISTAYMELSRLRSDDTAVYYCARGYYDILTGYYGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLAPPKPKDTLMISRTPEVTCVVADVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 458 aa, Homo sapiens
QVTLKESGPTLVKPTQTLTLTCTFSGFSLSTDGVGVGWIRQPPGKALEWLALIYGDDDKRYSPSLKSRLTITKDTSKNQVVLTMTNMDPVDTATYYCAHSISNYDILTGHWIAKTYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLAPPKPKDTLMISRTPEVTCVVADVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 458 aa, Homo sapiens
QVTLKESGPTLVKPTQTLTLTCTFSGFSLSTSRVGVGWIRQPPGKALEWLARIDWDDDKYYSTSLKTRLTISKDTSKNQVVLTMTNMDPVDTATYYCARTHTYYDILTGYSYHVYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLAPPKPKDTLMISRTPEVTCVVADVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 214 aa, Homo sapiens
EIVLTQSPGTLSLSPGERAMLSCRASQSVGNYLAWYQQKPGQAPRLLIYDASNRATGIPARFSGSGSGTDFTLTINSLEPEDFAVYYCQQRSNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 215 aa, Homo sapiens
DIVLTQSPGTLSFSPGERATLSCRASQNVANNFLAWFQHSPGQAPRLLIYDASNRATGIPARFSGSGSGTDFTLTISSLQTEDVAVYYCQQYYNLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 214 aa, Homo sapiens
DIRLTQSPSSLSASVGDRVTVACRASQNISSYLNWYQQKPGKAPKLLIYAASSLQSGVPSRFSGSGSGTDFTLTISSLQPEDFATYYCQQSYSTPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 214 aa, Homo sapiens
DIVMTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 214 aa, Homo sapiens
EIVLTQSPATLSLSPGERATLSCWTSQGVSGHLAWYQHKRGQAPRLLIYDTSNRATGVPARFSGSGSGTDFTLTISRLEPEDFAVYYCQQYGSSFWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 214 aa, Homo sapiens
EIVLTQSPATLSSSPGQRATLSCRASQSISSYLAWYVQKPGQAPSLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 216 aa, Homo sapiens
DIQLTQSPSTLSASVGDRVTITCRASQSISGWLAWYQQKPGRAPKLLIYKASSLESGVPSRFSGSGSGTDFTLTISSLQPEDIATYYCQQFDNLLLMYTFGQGTKLEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 214 aa, Homo sapiens
EIVLTQSPATLSLSPGERATLSCRANESVGNYLAWYQQKPGQAPRLLIYGASRRATGIPDRFSGSGSGTDFTLTISRLEPEDFAVYYCQQYGSSLLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 220 aa, Homo sapiens
EVVLTQSPDSLAVSLGERATINCKSSQSVLSSSNNKNYLAWYQQKPGQPPKLLIYWASTRESGVPDRFSGSGSGTDFTLTISSLQAEDVAVYYCQQYYSTPPTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 217 aa, Homo sapiens
QSALTQPASVSGSPGQSITISCTGTSSDVGGYNYVSWYQQHPGKAPKLMIYDVSKRPSGVSNRFSGSKSGNTASLTISGLQAEDEADYYCSSYTSSSTPVLFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 30 - protein, 216 aa, Homo sapiens
QSALTQPASVSGSPGQSITISCTGTSSDVGGYNYVSWYQQLPGKAPKLMIYEVSKRPSGVPDRFSGSKSGNTASLTISGLQAEDEADYYCSSYAGSSSWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 31 - protein, 216 aa, Homo sapiens
QSALTQPASVSGSPGQSITISCTGTSSDVGGYNYVSWYQQHPGKAPKLMIYDVNKRPSGVPDRFSGSKSANTASLTVSGLQAEDEADYYCSSYTAGGNWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 32 - protein, 216 aa, Homo sapiens
QSALTQPPSASGSPGQSVTISCTGTSSDVGHYNYVSWYQQHPGKAPKLIIFDVSDRPSGVSNRFSGSKSGNTASLTISGLQAEDEANYYCSSYTSRNTLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 33 - protein, 216 aa, Homo sapiens
QAVLTQPPSASGTPGQKVTISCSGSSSDIGSNNVNWYQQFPGTAPKLLIHSNNLRPSGVPVRFSGSKSGTSASLAISGLQSEDEADYYCSAWDDSVNGVAFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 34 - protein, 216 aa, Homo sapiens
QSVLTQPPSVSAAPGQKVTISCSGSSSNIGNNYVSWYQQLPGTAPKLLIYDNNKRPSGIPDRFSASKSGTSASLAISGLQAEDEADYYCQSYDSSLSGVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 35 - protein, 217 aa, Homo sapiens
QSALTQPASVSGSPGQSITISCTGTSSDIGRYNYVSWYQQRPGRAPKVIIYDVSSRPSGVSKRFSASKSGNTASLTISGLQAEDEAVYYCSSYTRITTLYVFGPGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 36 - protein, 216 aa, Homo sapiens
QPGLTQPPSASGTPGQRVTISCSGSSSNIGNNTVNWYQQLPGTAPRLLIYTNDQRPSGVPDRFSGSKSGTSASLAISGLQSEDEAQYYCSTWDDRQKAVVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 37 - protein, 216 aa, Homo sapiens
QSALTQPPSASGSPGQSVSISCIGSRSDIGYYNYVSWYQQHPGEAPKLIIFDVSKRPSGVPDRFSGSKSGNTASLTVSGLQPEDEADYYCASYGGRNNLLFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 38 - protein, 216 aa, Homo sapiens
QSALTQPPSASGSRGQSVSISCSGSRSDIGYYNYVSWYQQHPGKAPKLIIFDVNKRPSGVPDRFSGSKSGNTASLTVSGLQPEDEADYYCASYGGRNNLLFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 39 - protein, 10 aa, Homo sapiens
GYTFTSYGIS

SEQ ID NO: 40 - protein, 10 aa, Homo sapiens
GGTFSSYAIS

SEQ ID NO: 41 - protein, 10 aa, Homo sapiens
GYTFTDYYMH

SEQ ID NO: 42 - protein, 10 aa, Homo sapiens
GGTFSSYAIS

SEQ ID NO: 43 - protein, 10 aa, Homo sapiens
GYTFTSYAMH

SEQ ID NO: 44 - protein, 10 aa, Homo sapiens
GGTFSSYAIS

SEQ ID NO: 45 - protein, 10 aa, Homo sapiens
GYTFTSYAMH

SEQ ID NO: 46 - protein, 10 aa, Homo sapiens
GYTFTSYAMH

SEQ ID NO: 47 - protein, 10 aa, Homo sapiens
GYTFTGYYIH

SEQ ID NO: 48 - protein, 10 aa, Homo sapiens
GGTFSSYAIS

SEQ ID NO: 49 - protein, 10 aa, Homo sapiens
GFTFSSYAMS

SEQ ID NO: 50 - protein, 10 aa, Homo sapiens
GGSISGRNYY

SEQ ID NO: 51 - protein, 10 aa, Homo sapiens
GYTFTSYGIS

SEQ ID NO: 52 - protein, 10 aa, Homo sapiens
GFTFSSYGMH

SEQ ID NO: 53 - protein, 10 aa, Homo sapiens
GGTFSSYAIS

SEQ ID NO: 54 - protein, 10 aa, Homo sapiens
GYTFTSYYMH

SEQ ID NO: 55 - protein, 10 aa, Homo sapiens
GYTFTGYYMH

SEQ ID NO: 56 - protein, 12 aa, Homo sapiens
GFSLSTDGVGVG

SEQ ID NO: 57 - protein, 12 aa, Homo sapiens
GFSLSTSRVGVG

SEQ ID NO: 58 - protein, 16 aa, Homo sapiens
ISAYNGNTNYAQKLQG

SEQ ID NO: 59 - protein, 18 aa, Homo sapiens
RIIPILGIANYAQKFQGR

SEQ ID NO: 60 - protein, 17 aa, Homo sapiens
RINPNSGGTNYAQKFQG

SEQ ID NO: 61 - protein, 17 aa, Homo sapiens
GIIPIFGTANYAQKFQG

SEQ ID NO: 62 - protein, 17 aa, Homo sapiens
WINAGNGNTKYSQKFQG

SEQ ID NO: 63 - protein, 17 aa, Homo sapiens
WMNPNSGNTGYAQKFQG

SEQ ID NO: 64 - protein, 17 aa, Homo sapiens
WINAGNGNTKYSQKFQG

SEQ ID NO: 65 - protein, 17 aa, Homo sapiens
WINAGNGNTKYSQKFQG

SEQ ID NO: 66 - protein, 17 aa, Homo sapiens
WINPSSGGTNYAQKFQG

SEQ ID NO: 67 - protein, 17 aa, Homo sapiens
GISAYKGNTNYAQKLQG

SEQ ID NO: 68 - protein, 17 aa, Homo sapiens
AISGSGGSTYYADSVKG

SEQ ID NO: 69 - protein, 16 aa, Homo sapiens
SIYYSGSTYYNPSLKS

SEQ ID NO: 70 - protein, 17 aa, Homo sapiens
WISAYNGNTNYAQKLQG

SEQ ID NO: 71 - protein, 17 aa, Homo sapiens
VISHDGSNKYYADSVKG

SEQ ID NO: 72 - protein, 17 aa, Homo sapiens
RINPNSGGTNYAQKFQG

SEQ ID NO: 73 - protein, 17 aa, Homo sapiens
WINAGNGNTKYSQKFQG

SEQ ID NO: 74 - protein, 17 aa, Homo sapiens
WINPNSGGTNYAQKFQG

SEQ ID NO: 75 - protein, 16 aa, Homo sapiens
LIYGDDDKRYSPSLKS

SEQ ID NO: 76 - protein, 16 aa, Homo sapiens
RIDWDDDKYYSTSLKT

SEQ ID NO: 77 - protein, 17 aa, Homo sapiens
TYYDILTGYYYYYGMDV

SEQ ID NO: 78 - protein, 20 aa, Homo sapiens
DRFYDILTGYYTYYYGMDVW

SEQ ID NO: 79 - protein, 16 aa, Homo sapiens
EGYDFLTGYTLGGMDV

SEQ ID NO: 80 - protein, 16 aa, Homo sapiens
VDYDILTGYYMGYFDY

SEQ ID NO: 81 - protein, 16 aa, Homo sapiens
AYYDILTGYSVYGMDV

SEQ ID NO: 82 - protein, 15 aa, Homo sapiens
GHADILTGYLDAFDI

SEQ ID NO: 83 - protein, 15 aa, Homo sapiens
EMAVAGTYYYYGMDV

SEQ ID NO: 84 - protein, 16 aa, Homo sapiens
AYYDILTGYSVYGMDV

SEQ ID NO: 85 - protein, 16 aa, Homo sapiens
SYYDILTGYSRNPFDN

SEQ ID NO: 86 - protein, 21 aa, Homo sapiens
GRLAEYDILTGYYFNTWAFDI

SEQ ID NO: 87 - protein, 20 aa, Homo sapiens
EGQGYDILTGYYTRGYYFDY

SEQ ID NO: 88 - protein, 20 aa, Homo sapiens
DLANYDILTGYYRARGAFDI

SEQ ID NO: 89 - protein, 16 aa, Homo sapiens
ADYDILTGYPRGPVDY

SEQ ID NO: 90 - protein, 21 aa, Homo sapiens
DRSYYDILTGYYIPYYYGMDV

SEQ ID NO: 91 - protein, 16 aa, Homo sapiens
EGYDFLTGYTLGGMDV

SEQ ID NO: 92 - protein, 19 aa, Homo sapiens
DNLSYDILTGYSFGGWFDP

SEQ ID NO: 93 - protein, 15 aa, Homo sapiens
GYYDILTGYYGPFDY

SEQ ID NO: 94 - protein, 19 aa, Homo sapiens
SISNYDILTGHWIAKTYDY

SEQ ID NO: 95 - protein, 19 aa, Homo sapiens
THTYYDILTGYSYHVYFDY

SEQ ID NO: 96 - protein, 11 aa, Homo sapiens
RASQSVGNYLA

SEQ ID NO: 97 - protein, 12 aa, Homo sapiens
RASQNVANNFLA

SEQ ID NO: 98 - protein, 11 aa, Homo sapiens
RASQNISSYLN

SEQ ID NO: 99 - protein, 11 aa, Homo sapiens
RASQSVSSYLA

SEQ ID NO: 100 - protein, 11 aa, Homo sapiens
WTSQGVSGHLA

SEQ ID NO: 101 - protein, 11 aa, Homo sapiens
RASQSISSYLA

SEQ ID NO: 102 - protein, 11 aa, Homo sapiens
RASQSISGWLA

SEQ ID NO: 103 - protein, 11 aa, Homo sapiens
RANESVGNYLA

SEQ ID NO: 104 - protein, 17 aa, Homo sapiens
KSSQSVLSSSNNKNYLA

SEQ ID NO: 105 - protein, 14 aa, Homo sapiens
TGTSSDVGGYNYVS

SEQ ID NO: 106 - protein, 14 aa, Homo sapiens
TGTSSDVGGYNYVS

SEQ ID NO: 107 - protein, 14 aa, Homo sapiens
TGTSSDVGGYNYVS

SEQ ID NO: 108 - protein, 14 aa, Homo sapiens
TGTSSDVGHYNYVS

SEQ ID NO: 109 - protein, 13 aa, Homo sapiens
SGSSSDIGSNNVN

SEQ ID NO: 110 - protein, 13 aa, Homo sapiens
SGSSSNIGNNYVS

SEQ ID NO: 111 - protein, 14 aa, Homo sapiens
TGTSSDIGRYNYVS

SEQ ID NO: 112 - protein, 13 aa, Homo sapiens
SGSSSNIGNNTVN

SEQ ID NO: 113 - protein, 14 aa, Homo sapiens
IGSRSDIGYYNYVS

SEQ ID NO: 114 - protein, 14 aa, Homo sapiens
SGSRSDIGYYNYVS

SEQ ID NO: 115 - protein, 7 aa, Homo sapiens
DASNRAT

SEQ ID NO: 116 - protein, 7 aa, Homo sapiens
DASNRAT

SEQ ID NO: 117 - protein, 7 aa, Homo sapiens
AASSLQS

SEQ ID NO: 118 - protein, 7 aa, Homo sapiens
DASNRAT

SEQ ID NO: 119 - protein, 7 aa, Homo sapiens
DTSNRAT

SEQ ID NO: 120 - protein, 7 aa, Homo sapiens
DASNRAT

SEQ ID NO: 121 - protein, 7 aa, Homo sapiens
KASSLES

SEQ ID NO: 122 - protein, 7 aa, Homo sapiens
GASRRAT

SEQ ID NO: 123 - protein, 7 aa, Homo sapiens
WASTRES

SEQ ID NO: 124 - protein, 7 aa, Homo sapiens
DVSKRPS

SEQ ID NO: 125 - protein, 7 aa, Homo sapiens
EVSKRPS

SEQ ID NO: 126 - protein, 7 aa, Homo sapiens
DVNKRPS

SEQ ID NO: 127 - protein, 7 aa, Homo sapiens
DVSDRPS

SEQ ID NO: 128 - protein, 7 aa, Homo sapiens
SNNLRPS

SEQ ID NO: 129 - protein, 7 aa, Homo sapiens
DNNKRPS

SEQ ID NO: 130 - protein, 7 aa, Homo sapiens
DVSSRPS

SEQ ID NO: 131 - protein, 7 aa, Homo sapiens
TNDQRPS

SEQ ID NO: 132 - protein, 7 aa, Homo sapiens
DVSKRPS

SEQ ID NO: 133 - protein, 7 aa, Homo sapiens
DVNKRPS

SEQ ID NO: 134 - protein, 9 aa, Homo sapiens
QQRSNWPLT

SEQ ID NO: 135 - protein, 9 aa, Homo sapiens
QQYYNLPYT

SEQ ID NO: 136 - protein, 9 aa, Homo sapiens
QQSYSTPFT

SEQ ID NO: 137 - protein, 9 aa, Homo sapiens
QQRSNWPIT

SEQ ID NO: 138 - protein, 9 aa, Homo sapiens
QQYGSSFWT

SEQ ID NO: 139 - protein, 9 aa, Homo sapiens
QQRSNWPIT

SEQ ID NO: 140 - protein, 11 aa, Homo sapiens
QQFDNLLLMYT

SEQ ID NO: 141 - protein, 9 aa, Homo sapiens
QQYGSSLLT

SEQ ID NO: 142 - protein, 9 aa, Homo sapiens
QQYYSTPPT

SEQ ID NO: 143 - protein, 11 aa, Homo sapiens
SSYTSSSTPVL

SEQ ID NO: 144 - protein, 10 aa, Homo sapiens
SSYAGSSSWV

SEQ ID NO: 145 - protein, 10 aa, Homo sapiens
SSYTAGGNWV

SEQ ID NO: 146 - protein, 10 aa, Homo sapiens
SSYTSRNTLI

SEQ ID NO: 147 - protein, 11 aa, Homo sapiens
SAWDDSVNGVA

SEQ ID NO: 148 - protein, 11 aa, Homo sapiens
QSYDSSLSGVV

SEQ ID NO: 149 - protein, 11 aa, Homo sapiens
SSYTRITTLYV

SEQ ID NO: 150 - protein, 11 aa, Homo sapiens
STWDDRQKAVV

SEQ ID NO: 151 - protein, 10 aa, Homo sapiens
ASYGGRNNLL

SEQ ID NO: 152 - protein, 10 aa, Homo sapiens
ASYGGRNNLL

SEQ ID NO: 153 - protein, 285 aa, Homo sapiens
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 154 - protein, 309 aa, Mus
MDESAKTLPPPCLCFCSEKGEDMKVGYDPITPQKEEGAWFGICRDGRLLAATLLLALLSSSFTAMSLYQLAALQADLMNLRMELQSYRGSATPAAAGAPELTAGVKLLTPAAPRPHNSSRGHRNRRAFQGPEETEQDVDLSAPPAPCLPGCRHSQHDDNGMNLRNIIQDCLQLIADSDTPTIRKGTYTFVPWLLSFKRGNALEEKENKIVVRQTGYFFIYSQVLYTDPIFAMGHVIQRKKVHVFGDELSLVTLFRCIQNMPKTLPNNSCYSAGIARLEEGDEIQLAIPRENAQISRNGDDTFFGALKLL

SEQ ID NO: 155 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 156 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 157 - DNA, 5 bp, Artificial Sequence
  Kozak sequence
aaacg

SEQ ID NO: 158 - protein, 19 aa, Saccharomyces cerevisiae
MRFPSIFTAVLFAASSALA

SEQ ID NO: 159 - DNA, 64 bp, Artificial Sequence
  Kozak sequence
ggaaacgatgcgtttcccatccattttcaccgccgtcttgttcgctgcctcttctgccttggcc

SEQ ID NO: 160 - protein, 5 aa, Artificial Sequence
  peptide linker
PVGVV

SEQ ID NO: 161 - protein, 330 aa, Homo sapiens [sequence omitted]

SEQ ID NO: 162 - DNA, 642 bp, Artificial Sequence
  Codon optimized immunoglobulin
gacatcgtcatgacccagtccccagccaccttgtccttgtccccaggagagagagccaccttgagttgcagggcctcccaatccgtctcctcctacttggcctggtatcagcagaagcctggtcaggccccaagattgttgatctacgacgcctccaaccgtgccaccggtatcccagccagattctccggttctggttccgggacggacttcaccttgaccatctcctccttggagcctgaggacttcgccgtctactactgccaacagagatccaactggcctatcaccttcggtcaggggacgcgtttggagatcaagcgtaccgtcgccgccccatccgtcttcatcttcccaccttccgatgagcagttgaagtccggtaccgcttccgttgtttgtttgttgaacaacttctacccacgtgaggctaaggttcagtggaaggttgacaacgctttgcaatccggtaactcccaagaatccgttactgagcaggattctaaggattccacttactcattgtcctccactttgactttgtccaaggctgattacgagaagcacaaggtttacgcttgcgaggttacacatcagggtttgtcctccccagttactaagtccttcaacagaggagagtgt

SEQ ID NO: 163 - DNA, 1362 bp, Artificial Sequence
  Codon optimized immunoglobulin
caggttcagttggtccagtccggtgccgaggtcaagaagcctggtgcctccgtcaaggtctcttgcaaagcctccggaggaaccttctcctcctacgccatctcctgggtcagacaagccccaggtcaaggtttggagtggatgggtggaatcatcccaatcttcggaaccgccaactacgcccagaagttccagggtagagtcaccatcaccgctgacaagtccacctccaccgcctacatggagttgtcctccttgcgttccgaggacactgccgtctactactgcgcccgtgtcgactacgacatcttgaccggttactacatgggttacttcgactactggggtcagggaaccttggtcaccgtctcgagtgcttctactaagggaccatccgtttttccattggctccatcctctaagtctacttccggtggaaccgctgctttgggatgtttggttaaagactacttcccagagccagttactgtttcttggaactccggtgctttgacttctggtgttcacactttcccagctgttttgcaatcttccggtttgtactctttgtcctccgttgttactgttccatcctcttccttgggtactcagacttacatctgtaacgttaaccacaagccatccaacactaaggttgacaagaaggttgagccaaagtcctgtgacaagactcatacttgtccaccatgtccagctccagaattgttgggtggtccttccgtttttttggccccaccaaagccaaaggacactttgatgatctccagaactccagaggttacatgtgttgttgctgacgtttctcacgaggacccagaggttaagttcaactggtacgttgacggtgttgaagttcacaacgctaagactaagccaagagaggagcagtacaactccacttacagagttgtttccgttttgactgttttgcaccaggattggttgaacggaaaggagtacaagtgtaaggtttccaacaaggctttgccagctccaatcgaaaagactatctccaaggctaagggtcaaccaagagagccacaggtttacactttgccaccatccagagatgagttaactaagaaccaggtttccttgacttgtttggttaagggattctacccatccgacattgctgttgaatgggagtctaacggtcaaccagagaacaactacaagactactccacctgttttggactctgacggttcctttttcttgtactccaagttgactgttgacaagtccagatggcaacagggtaacgttttctcctgttccgttatgcatgaggctttgcacaaccactacactcaaaagtccttgtctttgtcccctggt